(19) United States
(12) Patent Application Publication
(10) Pub. No.: US 2016/0324992 A1
(43) Pub. Date: Nov. 10, 2016

(54) IDENTIFICATION AND TREATMENT OF VULNERABLE PLAQUES

(71) Applicant: OTTAWA HEART INSTITUTE RESEARCH CORPORATION, Ottawa (CA)

(72) Inventors: Katey J. RAYNER, Ottawa (CA); Terrence David RUDDY, Ottawa (CA); Denuja KARUNAKARAN, Ottawa (CA); Lihui WEI, Ottawa (CA)

(21) Appl. No.: 15/147,495

(22) Filed: May 5, 2016

Related U.S. Application Data
(60) Provisional application No. 62/157,758, filed on May 6, 2015.

(51) Int. Cl. A61K 51/04; A61K 47/48; A61K 31/4178

(52) U.S. Cl. CPC A61K 51/0453; A61K 31/4178; A61K 47/481

(57) ABSTRACT

A method of detecting an atherosclerotic plaque vulnerable to rupture in a subject comprises providing to the subject a labelled necrostatin or a derivative thereof, and visualizing the label, wherein a localization of the label in a plaque indicates the plaque is vulnerable to rupture. A method of detecting and treating an atherosclerotic plaque vulnerable to rupture comprises providing to the subject a labelled necrostatin or a derivative thereof, visualizing the label, wherein a localization of the label in a plaque indicates the plaque is vulnerable to rupture; and providing a necroptosis inhibitor or derivative thereof to the subject when the visualizing indicates that the plaque is vulnerable to rupture. The necroptosis inhibitor may comprise a necrostatin, such as Nec-1. The label may be a radiolabel such as ¹²³I. By visualizing plaques vulnerable to rupture, atherosclerotic plaques may be identified and treated in advance of rupture.

### CROSS REFERENCE TO RELATED APPLICATIONS

This application claims the benefit of priority of U.S. Provisional Patent Application No. 62/157,758 filed May 6, 2015, entitled “Diagnosis, prevention, and treatment of a cardiovascular disease” which is hereby incorporated by reference.

### FIELD

The present disclosure relates generally to diagnosis and treatment of a cardiovascular disease. More particularly, the identification and treatment of vulnerable plaques is described.

### BACKGROUND

Cardiovascular diseases are the primary cause of mortality in the US and Western Europe. Atherosclerotic diseases, including acute coronary syndrome and stroke, are leading causes of death and disability.

Atherosclerosis is a cardiovascular disease involving a maladaptive inflammatory process that is primarily driven by macrophage accumulation in blood vessel walls, which engulf modified-cholesterol forming foam cells. In atherosclerosis, these cells are unable to emigrate from the inflamed region and eventually undergo cell death, releasing damage-associated molecular patterns (DAMPs). As lesions progress and become more complex, a necrotic core forms, ultimately leading to plaque instability and rupture.

Arterial thrombosis is triggered by a ruptured atherosclerotic plaque, and can precipitate acute vascular events, responsible for a high mortality rate. Rupture-prone plaques, also referred to as vulnerable plaques, present a high risk to health. There is a medical need to slow or reverse the progression of vulnerable plaques.

Identification of patients at high risk for acute vascular events has relied mainly on the estimation of an individual's 10-year probability to present with or to die from an acute coronary or vascular problem with either the Framingham risk equation or the Systematic Coronary Risk Evaluation system. These systems are known to greatly underestimate the risk facing younger patients. Myocardial SPECT and/or stress echocardiography are frequently used to evaluate the hemodynamic significance of potential coronary artery stenosis. However, most vascular events are caused by the rupture or erosion of non-hemodynamically significant plaques, which by far outnumber flow-limiting lesions, and therefore the predictive value of these tests is limited. As such, there is a need for new techniques to identify patients at high risk.

Gan et al. (2014) previously described I-¹²³ labeled necrostatin-1 (Nec-1) uptake in stimulated necroptotic h9c2 cardiomyocytes. Lin et al. (2013) have studied aspects of macrophage necrosis in atherosclerosis. A need for identification and treatment of vulnerable atherosclerotic plaques exists.

It is desirable to provide compounds or methods for imaging, diagnosing or treating one or more cardiovascular disease, such as atherosclerosis and atherosclerotic plaques.

### SUMMARY

There is described herein a method, use, and composition aimed at obviating or mitigating at least one disadvantage of previous methods for imaging, diagnosing, treating, or otherwise addressing a cardiovascular disease, such as atherosclerosis and atherosclerotic plaques.

There is provided herein a method of detecting an atherosclerotic plaque vulnerable to rupture in a subject comprising providing to the subject a labelled necrostatin or a derivative thereof, and visualizing the label, wherein a localization of the label in a plaque indicates the plaque is vulnerable to rupture.

Further, there is provided a method of detecting and treating an atherosclerotic plaque vulnerable to rupture in a subject comprising: providing to the subject a labelled necrostatin or a derivative thereof, visualizing the label, wherein a localization of the label in a plaque indicates the plaque is vulnerable to rupture; and providing a necroptosis inhibitor or derivative thereof to the subject when the visualizing indicates that the plaque is vulnerable to rupture.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1A shows that oxidized LDL induces necroptotic cell death in macrophages. Bone-marrow-derived macrophages (BMDMs) were treated with 100 μg/mL oxLDL±zVAD.fmk±Nec-1 for 24 h and LDH release in the media was measured.

FIG. 1B shows cell death in response to oxLDL±zVAD.fmk in BMDMs from WT and Rip3^{-/-} mice.

FIG. 1C illustrates a Western blot analysis of RIP3 after treatment with oxLDL±zVAD.fmk ±Nec-1 for 8 hours.

FIG. 1D shows cell death in resting (M0), M1- and M2-polarized macrophages treated with oxLDL±zVAD.fmk for 24 h.

FIG. 1E illustrates Inhibition of oxLDL-induced cell death by Nec-1 in resting (M0), M1- and M2-polarized macrophages treated with oxLDL for 24 h.

FIG. 2A shows that a combination of oxLDL and DAMPs increases macrophage necroptosis; BMDMs were subjected to freeze thaw to generate necrotic DAMPs (NFT) which were added to cells with or without oxLDL±Nec-1 for 24 h and cell death was measured.

FIG. 2B shows BMDMs treated with necroptotic inducers LPS+zVAD.fmk for 24 h to generate necroptotic cell DAMPs (NC), which were added to cells with or without oxLDL±Nec-1 for 24 h and cell death measured.

FIG. 2C illustrates cell death in response to oxLDL±zVAD.fmk±necrotic freeze thaw (NFT) DAMPs in BMDMs from WT and Rip3^{-/-} mice.

FIG. 2D shows Western blot analysis of RIP3 after treatment with oxLDL±zVAD.fmk±Nec-1 with or without NFT DAMPs for 8 hours.

FIG. 3A shows that OxLDL induces the expression of RIP3 and MLKLBMDMs treated with 100 μg/ml oxLDL±zVAD.fmk for 24 h were analyzed for gene expression of RIP1, RIP3 and MLKL by qPCR.

FIG. 3B shows Western blot analysis of RIP3 and MLKL expression in BMDMs treated with oxLDL.

FIG. 3C shows gene expression of RIP1, RIP3 and MLKL assessed by qPCR in BMDMs at rest (M0) or polarized to an M1 or M2 phenotype.

FIG. 3D shows data from RAW macrophages transfected with RIP3-promoter or MLKL-promoter luciferase constructs and treated for 24 h with oxLDL before luciferase expression.

FIG. 3E shows RIP3 mRNA expression measured by qPCR in BMDMs treated with oxLDL±zVAD.fmk, in the presence or absence of pre-treatment with 50 μM DPI (Reactive Oxygen Species inhibitor).

FIG. 3F shows MLKL mRNA expression measured by qPCR in BMDMs treated with oxLDL±zVAD.fmk, in the presence or absence of pre-treatment with 50 μM DPI.

FIG. 3G shows cell death in BMDMs from WT and Casp 1^{-/-} mice in response to oxLDL±zVAD.fmk for 24 h.

FIG. 3H shows cell death in response to oxLDL ±zVAD in BMDMs treated with or without the caspase-1 inhibitor zYVAD.fmk (20 uM).

FIG. 4A illustrates that Nec-1 therapy decreases atherosclerotic lesion progression and markers of instability in ApoE^{-/-} mice. ApoE^{-/-} mice were fed a Western diet for 6 weeks prior to implantation of time-release pellets containing placebo or Nec-1s (2 mg/kg/day). Morphometric analysis of atherosclerosis is shown.

FIG. 4B shows en face lesion area measured in placebo and Nec-1s treated mice and is represented as lesion area as a % of total aorta area.

FIG. 4C shows lesion area in the aortic sinus in placebo and Nec-1s treated mice is represented as total area in μm².

FIG. 4D shows necrotic core area staining within aortic sinus lesions, quantified with Image J.

FIG. 4E shows smooth muscle actin staining within aortic sinus lesions, quantified with Image J.

FIG. 5A shows that radiolabeled Nec-1 can be utilized to visualize atherosclerotic plaques in ApoE^{-/-} mice. Left side shows the chemical structure of the 7-¹²³I-Nec-1 tracer; central panels are representative images of aortic en face (no stain and oil red-O [ORO] stain); right side panel shows a representative autoradiogram from mice injected with ¹²³I-labelled Nec-1 tracer.

FIG. 5B shows the correlation of lesional uptake of ORO compared to ¹²³I-Nec-1 (n=9).

FIG. 5C illustrates autoradiographic images from mice injected with non-radioactive Cl-Nec-1 compound 1.5 h prior to being injected with radiolabelled ¹²³I-Nec-1 tracer.

FIG. 5D shows lesional uptake for two groups of mice, indicating radiolabeled Nec-1 uptake is specific.

FIG. 6A shows up-regulation of necroptotic genes in unstable atherosclerosis, with mRNA expression of RIP3 in carotid plaque from asymptomatic or symptomatic patients versus macroscopically disease-free control arteries.

FIG. 6B shows up-regulation of necroptotic genes in unstable atherosclerosis, with mRNA expression of MLKL in carotid plaque from asymptomatic or symptomatic patients, or from macroscopically disease-free control arteries.

FIG. 7 illustrates HPLC chromatograms of 7-¹²³I-O-Nec-1 (radiomatic trace) (Panel A); and 7-¹²⁷I-O-Nec-1 (UV trace at 280 nm) (Panel B).

FIG. 8 shows biodistribution of 7-¹²³I-O-Nec-1 in C57BL/6 mice at 1 hr and 2 hr post injection (p.i.). The mice (19-21 g) were injected intravenously with 5.3-6.7 MBq of 7-¹²³I-O-Nec-1 in water. The error bars represent standard deviation (SD).

FIG. 9 shows comparison of aortic lesion uptake in 4 month old ApoE^{-/-} mice fed with a western high fat diet (HFD) for 2 months (4 mo ApoE with HFD), 4 month old ApoE^{-/-} mice fed with a chow diet for 2 months (4 mo ApoE), and 4 month old C57BL/6 control mice (4 mo control) (n=3 per group).

FIG. 10 shows en face, Oil Red O and autoradiography images of a 4 month old ApoE^{-/-} mouse fed with a western high fat diet (HFD) for 2 months (Panel A); and a 4 month old C57BL/6 control mouse (Panel B).

FIG. 11 shows correlation of lesion area measured from en face, Oil Red O (upper panel) and autoradiography (lower panel) images. The three spots at lower percentages are from the 4 month ApoE^{-/-} mice fed with a chow diet, and the three spots at higher percentage are from the 4 month ApoE^{-/-} mice fed with HFD.

FIG. 12 illustrates an up-regulation of necroptotic genes in unstable atherosclerosis. Panel A shows mRNA expression of RIP3 and MLKL in carotid endarterectomies (plaque) or macroscopically disease-free control arteries (normal). Panel B shows RIP3 and MLKL mRNA expression in plaque samples from Panel A classified as asymptomatic (Stable) or symptomatic patients (Unstable). Panel C shows Immunohistochemical analysis of phosphorylated MLKL in human coronary arteries with early lesions.

FIG. 13 shows that oxidized LDL induces necroptotic cell death in macrophages. (Panel A) Bone-marrow-derived macrophages (BMDMs) were treated with 100 μg/mL oxLDL±zVAD.fmk±Nec-1 for 24 h and LDH release in the media was measured. (Panel B) Cell death in response to oxLDL±zVAD.fmk in BMDMs from WT and Rip3−/− mice. (Panel C) Western blot analysis of RIP3 after treatment with oxLDL±zVAD.fmk±Nec-1 for 8 hours. (Panel D) Western blot analysis of pMLKL after treatment with oxLDL for 12 h or oxLDL±zVAD.fmk for 8 h. (Panel E) Electron microscopy ultrastructural analysis of control and oxLDL-treated macrophages. (Panel F) BMDMs were subjected to freeze thaw to generate necrotic DAM Ps which were added to cells with or without oxLDL±Nec-1 for 24 h and cell death measured. (Panel G) Cell death in response to oxLDL±zVAD.fmk±necrotic freeze thaw DAMPs in BMDMs from WT and Rip3−/− mice. (Panel H) Western blot analysis of RIP3 after treatment with oxLDL±zVAD.fmk±Nec-1 with or without DAMPs for 8 hours. (Panel I) Cell death in BMDMs from WT and Casp1−/− mice in response to oxLDL±zVAD.fmk for 24 h. (Panel J) Cell death in response to oxLDL±zVAD in BMDMs treated with or without the caspase-1 inhibitor zYVAD.fmk (20 μM).

FIG. 14 shows that oxLDL induces the expression of RIP3 and MLKL. In Panels A and B, BMDMs treated with 100 ug/mloxLDL±zVAD.fmk for 3, 6, 12 or 24 h were analyzed for gene expression of RIP3 and MLKL by qPCR. In Panels C and D show Western blot analysis of RIP3 and MLKL expression in BMDMs treated with oxLDL. Panel E shows BMDMs pre-incubated with 50 μM DPI and then treated with oxLDL or oxLDL+zVAD.fmk prior to measuring ROS levels. Panel F shows RIP3 or MLKL mRNA expression measured by qPCR in BMDMs treated with oxLDL±zVAD.fmk, in the presence or absence of pre-treatment with 50 uM DPI. Panels G and H show RAW macrophages were transfected with RIP3-promoter or MLKL-promoter luciferase constructs and treated in the presence or absence of pre-treatment with 50 uM DPI.

FIG. 15 shows radiolabeled Nec-1 can be utilized to visualize atherosclerotic plaques in Apoe−/− mice. (Panel A) Left: chemical structure of 7-¹²³I-Nec-1 tracer. Images of aortic en face (no stain and oil red-O [ORO] stain) and autoradiography from mice injected with ¹²³I-labelled Nec-1 tracer are shown. (Panel B) Mice were injected with non-radioactive CI-Nec-1 compound 1.5 h prior to being injected with radiolabelled ¹²³I-Nec-1 tracer, subjected to autoradiography (images shown) and lesional uptake quantification (n=3 mice per group). (Panel C) Correlation of lesional uptake of ORO compared to ¹²³I-Nec-1 (n=9).

FIG. 16 shows Nec-1 therapy decreases atherosclerotic lesion progression and markers of instability in Apoe−/− mice. (Panel A) Apoe−/− mice were fed a Western diet for 6 weeks prior to implantation of time-release pellets containing placebo or Nec-1s (2 mg/kg/day). (Panel B) En face lesion area was measured in placebo and Nec-1s treated mice and is represented as lesion area as a % of total aorta area. (Panel C) Lesion area in the aortic sinus in placebo and Nec-1s treated mice is represented as total area in μm². (Panel D) Necrotic core area within aortic sinus lesions. Panels E, F and G show Immunohistochemical staining of smooth muscle-a actin, CD68, and phosphorylated MLKL. (Panel H) Serum IL-1μ in mice from placebo or Nec-1 treated groups was measured at sacrifice by ELISA.

FIG. 17 shows biodistribution of labeled Nec-3 and Nec-1 tracers in female C57BL/6 mice. Panel A shows comparison of 3 Nec-3 tracers and Panel B shows comparison of Nec-1 and Nec-3 tracers.

FIG. 18 shows en face (first column), Oil Red O (second column), and autoradiography (third column) aortic images from an ApoE−/− mouse injected with 3′-¹²³I-Nec-3R (Panel A), an ApoE−/− mouse injected with 3′-¹²³I-Nec-3R after consuming a high fat diet (Panel B), a wild type C57BL/6 mouse injected with 3′-¹²³I-Nec-3R (Panel C), and an ApoE−/− mouse injected with 8-¹²³I-Nec-3R following a high fat diet (Panel D).

### DETAILED DESCRIPTION

Generally, the present disclosure provides methods of treatment, prevention, and detection of a cardiovascular disease, such as atherosclerosis. Compositions and uses for treatment, prevention and detection are also described.

Necroptosis, or programmed necrosis, is a cell death pathway involving kinases RIP1 and RIP3. Unlike apoptosis, necroptosis is a pro-inflammatory form of cell death. Necroptosis inhibitors such as necrostatins are described, for example in U.S. patent application Ser. No. 14/352,960 (U.S. Publication No. U.S. 2014/0357570 A1) entitled Compositions Comprising Necrosis Inhibitors; U.S. patent application Ser. No. 13/665,263 (U.S. Publication No. U.S. 2013/0158024 A1) entitled Tricyclic Necrostatin Compounds; and U.S. Patent Application Ser. No. 14/214,360 (U.S. Publication No. U.S. 2014/0323489 A1) entitled Hybrid Necroptosis Inhibitors. Also, see for example Gao et al., 2010; Lin et al., 2013; Xie et al., 2013).

As referred to herein, cardiovascular diseases include diseases of the heart or blood vessels, such as atherosclerosis, unstable plaque in an artery, acute inflammation, chronic inflammation, inflammatory sepsis, ischemic heart disease, stroke, hypertensive heart disease, rheumatic heart disease, aortic aneurysms, cardiomyopathy, atrial fibrillation, congenital heart disease, endocarditis, peripheral artery disease, and others. Atherosclerosis may underlie more than one other cardiovascular disease, and may be associated with high blood pressure, smoking, diabetes, lack of exercise, obesity, high blood cholesterol, poor diet, stress, or excessive alcohol.

A method is described herein for detecting an atherosclerotic plaque vulnerable to rupture in a subject. The method comprises providing to the subject a labelled necrostatin or a derivative thereof, and visualizing the label. A localization of the label in a plaque indicates that plaque is vulnerable to rupture. The label may be a radiolabel. The necrostatin to be used may comprise necrostatin-1 (Nec-1), necrostatin-3 (Nec-3), necrostatin-4 (Nec-4), necrostatin-5 (Nec-5) or necrostatin-7 (Nec-7). Exemplary necrostatins are: ¹²³I—labelled or ¹⁸F-labelled Nec-1, ¹²³I—labelled or ¹⁸F-labelled Nec-3, or a derivative thereof. For example, the labelled necrostatin may be 7-¹²³I-O-Nec-1. The localization of label in a plaque can indicate an active necrotic plaque comprising macrophages. The labelled Nec-1, Nec-3, or a derivatives of these can be used for single photon emission computed tomography (SPECT) imaging. Alternatively, when the labelled necrostatin comprises an ¹⁸F-labelled Nec-1, Nec-3 or a derivative thereof, these may be used for positron emission tomography (PET) imaging. The extent of the localization of the label in the plaque of the subject can be compared with the accumulation of the label at a level indicative of atherosclerosis. Further, the extent of the localization of the label in the plaque of the subject can be compared with the accumulation of the label at a level indicative of a ruptured plaque.

A method is described herein for detecting and treating an atherosclerotic plaque vulnerable to rupture in a subject. The method comprises providing to the subject a labelled necrostatin or a derivative thereof; visualizing the label, wherein a localization of the label in a plaque indicates the plaque is vulnerable to rupture; and providing a necroptosis inhibitor or derivative thereof to the subject when the visualizing indicates that the plaque is vulnerable to rupture.

In this method, the necroptosis inhibitor may be Nec-1. The necroptosis inhibitor or derivative thereof may comprise a conjugate of necrostatin linked to an anti-inflammatory agent for targeted delivery of the anti-inflammatory agent to an atherosclerotic plaque. The anti-inflammatory agent may be one that reduces or inhibits RIP1, RIP3, or MLKL expression. The necroptosis inhibitor may comprise a small molecule, a microRNA (miRNA), a small interfering RNA (sRNA), or a short hairpin RNA (shRNA). The necroptosis inhibitor may be one that reduces production, activity or expression of a RIP3 or a RIP1 kinase, or of MLKL in macrophages. In this method, the subject may be one that already is known to have atherosclerosis. The subject may be a person who is known to have consumed a high fat diet. Treating the atherosclerotic plaque in this way may prevent a thrombotic event, or may delay or reverse the progression of the plaque. The necroptosis inhibitor may be one that reduces production or activity of the RIP3 kinase in macrophages.

A method is described for treating or preventing cardiovascular disease in a subject comprising administering an effective amount of a necroptosis inhibitor to the subject. The necroptosis inhibitor may comprise a small molecule, a microRNA (miRNA), a small interfering RNA (sRNA), or a short hairpin RNA (shRNA). The necroptosis inhibitor may comprise a necrostatin, for example Nec-1, Nec-3, Nec-4, Nec-5 or Nec-7.The necroptosis inhibitor may be one that reduces production, activity, or expression of RIP3 or RIP1 kinases, or of MLKL in macrophages.

The cardiovascular disease may, for example, comprise atherosclerosis, unstable plaque in an artery, acute inflammation, chronic inflammation, or inflammatory sepsis. The method described may be for preventing a thrombotic event in the subject, or it may be for delaying, preventing, or reversing the progression of an atherosclerotic plaque to a vulnerable plaque. The necroptosis inhibitor may be one that reduces production or activity of a RIP3 or RIP1 kinase or MLKL in macrophages. When the necrostatin is Nec-1, it may be derivatized at postion-7 to increased necroptosis inhibitory activity. In some instances, the subject may have consumed a high fat diet.

The method for detecting an atherosclerotic plaque in a subject, described herein, may comprise providing to the subject a labelled necrostatin and visualizing the label.

A method is also described herein for detecting a cardiovascular disease in a subject comprising providing to the subject a labelled necrostatin and detecting an accumulation of the label. The label may be a radiolabel. The labelled necrostatin may comprise Nec-1, Nec-3, Nec-4, Nec-5 or Nec-7. The labelled necrostatin may comprise ¹²³I-labeled Nec-1 or a derivative thereof. Further, the labelled necrostatin may comprise 7-¹²³I-O-Nec-1. Detecting an atherosclerotic plaque may comprise detecting a plaque in advance of formation of an atherosclerotic lesion. Further, detecting the atherosclerotic plaque may comprise detecting the size of an atherosclerotic lesion area. Detecting an atherosclerotic plaque may comprise detecting a vulnerable plaque in advance of rupture.

In the method described, the labeled necrostatin may comprise a necrostatin-1 derivative for SPECT imaging, or the labelled necrostatin may comprise 7-¹⁸F-O-Nec-1 for PET imaging.

A method for targeted delivery of an anti-inflammatory agent to an atherosclerotic plaque may comprise delivery of the anti-inflammatory agent coupled to a necroptosis inhibitor to a subject in need thereof. The necroptosis inhibitor may be a necrostatin.

A conjugate is described herein, comprising necrostatin linked to an anti-inflammatory agent for targeted delivery of the anti-inflammatory agent to an atherosclerotic plaque. The anti-inflammatory agent may be one that reduces or inhibits RIP1, RIP3, or MLKL expression.

A method of diagnosing atherosclerosis may comprise detecting elevated levels of necroptosis in a subject. Detecting elevated levels of necroptosis may comprise delivering labelled necrostatin to the subject and evaluating labelled necrostatin in vivo to detect elevated levels within vascular lesions. The diagnosing of advanced atherosclerotic lesion areas means lesion areas which are susceptible to rupture of unstable plaque.

Uses are described for a necroptosis inhibitor for treating or preventing cardiovascular disease in a subject, or for preparation of a medicament for treating or preventing cardiovascular in a subject. The necroptosis inhibitor may comprise a small molecule, a microRNA (miRNA), a small interfering RNA (sRNA), or a short hairpin RNA (shRNA). The necroptosis inhibitor may comprise a necrostatin, for example Nec-1, Nec-3, Nec-4, Nec-5 or Nec-7. The necroptosis inhibitor described may reduce production or activity of RIP1 or RIP3 kinase or MLKL in macrophages. In the uses described, the cardiovascular disease may comprise atherosclerosis, unstable plaque in an artery, acute inflammation, chronic inflammation, or inflammatory sepsis. The use may be for preventing a thrombotic event in the subject, or may be for delaying, preventing, or reversing the progression of an atherosclerotic plaque to a vulnerable plaque. The necroptosis inhibitor may be one that reduces production or activity of a RIP3 or RIP3 kinase, or MLKL in macrophages.

The Nec-1 may be one that is derivatized at any appropriate position on the molecule, such as at postion-7, to increased necroptosis inhibitory activity. The subject in such uses may be one that has consumed a high fat diet.

A composition for use in treating or preventing cardiovascular in a subject is described herein, said composition comprising a necroptosis inhibitor and a pharmaceutically acceptable carrier. In the composition the necroptosis inhibitor may comprise a necrostatin.

Macrophages undergo necroptosis in response to atherogenic stimuli in the vessel wall, and this process underlies necrotic core formation and plaque instability. Atherogenic ligands activate necroptosis in macrophages, which in turn, contributes to atherosclerotic plaque and necrotic core formation.

It has been found that necrostatins such as Nec-1, Nec-3, Nec-4, Nec-5, Nec-7 can be used for imaging, diagnosis, and therapy, and may be used to treat cardiovascular diseases, for example reducing incidence or severity of atherosclerosis, and/or treating unstable plaque in atherosclerosis.

It has been found that necroptosis plays a role in atherosclerosis and other cardiovascular disorders. For example, necroptosis has a role in the advancement of plaques. Necroptosis is initiated through the interaction of RIP1 and RIP3, after which downstream effectors induce membrane rupture and the release of danger-associated molecular patterns (DAMPs) which induce inflammatory response.

Necrostatin-1 (Nec-1) is an inhibitor of receptor-interacting protein kinase 1 (RIP1 or RIPK-1). Thus Nec-1 is an inhibitor of necrotic cell death. Nec-1 specifically inhibits the interaction of RIP1-RIP3, thereby inhibiting necroptosis. It is shown herein that necrostatins, such as Nec-1, can be used to prevent or delay the formation of vulnerable atherosclerotic plaques. Reduction and/or reversal of the progression of plaques to a vulnerable state may be accomplished by inhibiting the processes underlying programmed necrosis.

In certain embodiments described herein, radiolabeled Nec-1 (¹²³I-Nec-1) was prepared and demonstrated to have utility in visualizing the plaques to determine vulnerable plaques. A necrotic core within an atherosclerotic plaque renders it susceptible to rupture and subsequent thrombus and myocardial infarction. Hence it is important to be able to identify such a vulnerable plaque, and to intervene therapeutically to stabilize or reverse the vulnerability of the plaque.

It has been found and is described herein that macrophage necroptosis is induced by atherogenic ligands and promotes atherosclerotic lesion vulnerability.

Design, synthesis and evaluation of a I-¹²³ labeled necrostatin-1 derivative is described herein as a SPECT imaging agent targeting necroptosis.

Atherogenic ligand (oxLDL) induces cells to undergo necroptosis via activation of RIP3 and MLKL expression which is exacerbated by inhibition of caspases. Within the plaque, macrophages with a pro-inflammatory phenotype show increased susceptibility to cell death (induced by oxLDL) and have higher expression of RIP3 and MLKL than resting or M2 macrophages, indicating necroptosis, rather than apoptosis. Both RIP3 and MLKL were found to be upregulated in human carotid arteries with atherosclerosis as well as carotid plaques from individuals with symptomatic carotid disease. Damage-associated molecular patterns, or DAMPs, promote the inflammatory response and were found to exacerbate oxLDL-induced cell death and is dependent on RIP3.

A therapeutic strategy is described herein for reversing plaque vulnerability. Targeting necroptosis pathways may reverse or reduce both the development of plaques and the vulnerability of the plaques.

The cell death exacerbated by DAMPs and activation of RIP3 by phosphorylation were inhibited by the necroptosis inhibitor Nec-1, illustrating that necrosis inhibitors, for example Nec-1, may serve as a ligand for resisting progression of plaque vulnerability.

Necrosis was found to be inhibited by Nec-1 in Apo E^{-/-} mice with established atherosclerosis which were subjected to a western diet for 4 weeks and administered either placebo or Nec-1s (2 mg/kg/day) for 6 weeks. This was seen by reduced en face lesion areas after 6 weeks and reduced lesion necrotic core by 62%, which is an indicator of plaque stability.

The necroptosis pathway can be utilized to visualize and identify plaque vulnerability. In the Examples below, radiolabeled Nec-1 (7-¹²³I-O-Nec-1) is confirmed in vivo to be useful as a SPECT imaging agent for visualizing advanced atherosclerotic lesion area, and was shown to be specific, in an ex vivo autoradiography study with Apo E^{-/-} mice. Other labeled tracers are prepared and described herein, such as 3′-¹²³I-Nec-3R, 4′-¹²³I-Nec-3R, and 8-¹²³I-Nec-3R, for example (R isomers). Derivatives useful in PET are also described, using ¹⁸F-fluorodeoxyglucose (FDG), a radiolabled glucose analog, as the label. For example, 7-¹⁸F-O-Nec-1 can be used as a PET tracer.

The compound 7-¹²³I-O-Nec-1 was found to be stable after incubating with rat serum at 37° C. for 24 hours. Further, biodistribution studies showed fast renal and liver/gastrointestinal clearance of this compound, used as a tracer.

¹²³I may be used for SPECT imaging. ¹²³I has a half-life of 13.2 hours and optimal gamma energy (159 keV). For PET imaging, ¹¹C and ¹⁸F may be used.

### EXAMPLES

The following Examples are illustrative, but should not be construed as limiting.

### Example 1

### Macrophage Necroptosis is Induced by Atherogenic Ligands and Promotes Atherosclerotic Lesion Vulnerability.

### Overview

Atherosclerosis is a disease of maladaptive inflammation driven primarily by macrophages, and the atherosclerotic plaque grows when the rate of macrophage accumulation (via recruitment and proliferation) exceeds that of removal (via cell death and egress). As lesions progress and become more complex, a necrotic core forms and ultimately underlies the instability that drives plaque rupture and downstream adverse clinical events. Necroptosis, also known as ‘programmed necrosis’, is an emerging cell death pathway involving kinases RIP3 and MLKL that in contrast to apoptosis, induces a pro-inflammatory state. Atherogenic ligands induced necroptotic cell death in macrophages which can be blocked using a necroptosis inhibitor Nec-1. Oxidized LDL treatment of macrophages increases the expression of necroptotic genes RIP3 and MLKL through activation of the promoter region and induces phosphorylation of RIP3—a critical step in the execution of necroptosis. In a mouse model of established atherosclerosis, Nec-1 intervention in ApoE−/− mice reduced lesion size and markers of plaque instability, including reduced necrotic core formation. A radiotracer targeting the necroptosis pathway is described, showing that ¹²³I-Nec-1 specifically localizes to atherosclerotic plaques, and uptake is tightly correlated to lesion areas by ex vivo nuclear imaging. Finally, in humans with unstable carotid atherosclerosis, expression of necroptotic genes is elevated in comparison to those with stable atherosclerosis, indicating that the necroptotic pathway is activated in vulnerable plaques. These findings offer molecular insight into the inflammatory cell death that drives necrotic core formation that underlies unstable atherosclerosis development. This pathway can be used as both a diagnostic and therapeutic tool for the treatment of atherosclerosis (for example, unstable atherosclerosis), or other cardiovascular diseases.

### Introduction

For decades, it has been understood that the presence of a necrotic core within an atherosclerotic plaque renders it susceptible to rupture and subsequent thrombus, myocardial infarction or stroke. A so-called ‘vulnerable plaque’ is replete with macrophages, T-lymphocytes, lipids and cholesterol crystals, and contains a large necrotic core covered by a thin fibrous cap. While the processes that underlie the initiation of inflammatory fatty lesions within the arterial wall are well understood, the mechanisms by which these benign lesions develop into rupture-prone culprit lesions are not and yet are the most urgent for therapeutic targeting.

Atherosclerosis is initiated by the accumulation of excess low-density lipoprotein cholesterol that becomes trapped in the subendothelial space, where it becomes modified in the oxidant rich environment. According to the so-called “oxidation hypothesis”, oxidized LDL (oxLDL) activates innate immune cells, particularly macrophages, to engulf the modified-self LDL using constitutively expressed scavenger receptors on their cell surface, triggering the activation of the inflammasome. OxLDL can be cytotoxic and can induce apoptosis of macrophages and smooth muscle cells, which can be subsequently cleared away by healthy macrophages by a process termed efferocytosis. In the atherosclerotic plaque, efferocytosis is necessary to clear away the debris generated by dying cells in order to prevent the activation of inflammation, which is suppressed by the interaction of phagocytes and apoptotic cells. However, should efferocytosis become defective, apoptotic cells undergo secondary necrosis, promoting the uncontrolled release of inflammatory mediators, proteases and coagulation factors, which are all factors that promote plaque vulnerability.

In the last decade, the understanding of cell death has increased with the discovery and characterization of the pathway of programmed necrosis, or ‘necroptosis’ (Vandenabeele et al., 2010). Apoptosis and necroptosis have evolved as counterbalances in the first line of defense against inflammatory stimuli, either exogenous or self-derived. When apoptosis is inhibited or overwhelmed, necroptosis is initiated through the interaction of RIP1 and RIP3, after which their downstream effectors induce membrane rupture and the release of danger-associated molecular patterns (DAMPs). When pro-apoptotic caspase-8 is rendered inactive either through synthetic or naturally occurring inhibitors, RIP1 and RIP3 become phosphorylated, MLKL is recruited and activated by RIP3, and plasma membrane integrity is lost, releasing cellular contents. While necroptosis and apoptosis share many overlapping factors, the small molecule necrostatin-1 (Nec-1) uniquely inhibits the interaction of RIP1-RIP3 and subsequent downstream effectors of necroptosis, thus, Nec-1 can generally be considered a specific inhibitor of necroptosis rather than apoptosis. Although the upstream stimuli of necroptosis are only just emerging, it is now appreciated that a wide variety of both self and non-self ligands can trigger necroptosis and this pathway is highly active during physiological and pathophysiological processes.

Given the extensive necrotic core that accompanies advanced atherosclerotic lesions, and the observation that necroptosis can be activated by modified self DAMPs, it was evaluated whether atherogenic ligands can trigger necroptotic cell death in macrophages within the atherosclerotic plaque. Described herein are the findings that oxLDL and DAMPs induce necroptotic cell death in macrophages, and this can be inhibited in vivo to reduce atherosclerotic lesion vulnerability. A molecular imaging tool targeting necroptosis is also described, that can be used to visualize atherosclerosis lesions in vivo, and demonstrate that necroptosis markers are elevated within lesions in patients with atherosclerosis.

### Materials and Methods

Reagents. HI-TBAR oxidized human LDL (BT-910X) and human LDL (BT-903) was purchased from Biomedical Technologies Inc (USA). IFNγ, IL-4, M-CSF and zVAD.fmk were purchased from R & D Systems (USA). Lipopolysaccharide (LPS), Necrostatin-1 and Diphenyleneiodonium (DPI) were obtained from Sigma Aldrich.

Mice. C57BL6 wildtype (WT) and ApoE^{-/-} and Casp1−/− mice were purchased from Jackson Laboratories. Rip3^{-/-} mice were obtained from Genentech (San Francisco, Calif.).

Bone-marrow derived macrophages. Bone-marrow derived macrophages (BMDMs) were isolated from femurs of adult VVT or Rip3^{-/-} mice and differentiated into macrophages using DMEM supplemented with 10% FBS and 1% penicillin-streptomycin plus either 20% L929 conditioned media or 20 ng/mL mouse M-CSF for 7-10 days. To polarize macrophages to M1 or M2, BMDMs were incubated on day 7 with 1 μg/mL LPS and 100 ng/mL IFNγ or 10 ng/mL IL-4 respectively for 24 h. Damage Associated Molecular Patterns (DAMPs) were obtained by subjecting BMDMs to 3×30 min freeze-thaw cycle or treatment with 100 ng/mL LPS and 50 μM zVAD.fmk for 24 hours, after which the media was collected and added to naïve cells at indicated ratios.

Cell viability assays. Cell death was determined by measuring lactate dehydrogenase (LDH) release into the media. Briefly, cells were treated with 100 μg/ml oxLDL in the presence or absence of 25 μM zVAD.fmk or 50 μM necrostatin-1 (Nec-1) for 24 hours and media was collected and centrifuged to pellet cell debris. The amount of LDH in media was measured in a kinetic assay by adding PBS containing 0.02% NADH and 0.03% sodium pyruvate and measuring absorbance at 340 nm for 10 minutes at 1 minute intervals. The slope of the curve provides a measure of cell death, which was expressed as fold change relative to control.

RNA isolation and quantitative real-time PCR. BMDMs were treated with control media with or without oxLDL or oxLDL+zVAD.fmk for the indicated time points with or without 1 h pre-treatment with 50 μM DPI. Alternatively, BMDMs were polarized to M0, M1 or M2 prior to RNA isolation, as described above. Total RNA was isolated using TRIzol reagent (Invitrogen) as per manufacturer's instructions and cDNA was synthesized using iScript Reverse Transcription kit (BioRad™). Quantitative real-time PCR was performed in triplicate using either the Sso Advanced Universal SYBR Green Supermix (Biorad) or Taqman™ Gene Expression Assay and mRNA level of target genes was normalized to HPRT or β-actin house keeping genes.

Western blot analysis. For western blot analysis of RIP3, cells were lysed in 1.25×sample buffer (83 mM Tris pH 6.8, 6.7% SDS, 13.3% Glycerol, 1.3% β-mercaptoethanol, 0.03% bromophenol blue) and boiled at 100° C. for 5 minutes prior to being subjected to SDS-PAGE and western blot analysis as previously described (Robinson et al., 2012). PVDF membranes were blocked with 5% skim milk, followed by incubation with RIP3 (ProSci Inc, 1:500) or HSP90 (Santa Cruz Biotechnology, 1:1000) antibodies. For analysis of MLKL protein, cells were lysed in ice-cold M2 lysis buffer (50 mM NaF, 20 mM Tris, pH 7.0, 0.5% NP40, 250 mM NaCl, 3 mM EDTA, 3 mM EGTA, supplemented with Roche protease and phosphatase inhibitor cocktails) for 30 mins before centrifugation to pellet insoluble component. Samples were mixed 3:1 with 4×sample loading buffer (Bio-Rad) prior to SDS PAGE and western blot analysis. PVDF membranes were blocked in 5% BSA and probed with MLKL (Millipore, 1:500) or GAPDH (Millipore, 1:1000) antibodies. Goat anti-mouse (1:2500) or anti-rabbit (1:5000) or anti-rat (1:2500) IRDye® secondary antibodies (Rockland) were utilized. The protein bands were visualized using Odyssey Infrared Imaging System (LI-COR Biotechnology).

Promoter Luciferase Assay. Luciferase reporter constructs containing the human MLKL and RIP3 promoters were purchased from SwitchGear Genomics (Carlsbad, CA). RAW264.7 (ATCC) macrophages were plated in 24-well plates in 10% FBS DMEM and transfected using Lipofectamine 2000 (Life Technologies) with MLKL-promoter or RIP3-promoter plasmids (renilla-based luciferase reporter) together with pGL3 basic (firefly-based reporter, Promega) to normalize for transfection efficiency. Twenty-four hours after transfection, cells were treated with or without 100 μg/ml oxLDL as above, for an additional 24 hours. Cells were harvested in lx passive lysis buffer and luciferase measured using the Dual Luciferase Assay (Promega) according to manufacturer's instructions. All experiments were performed in quadruplicate technical replicates at least 3 times.

Atherosclerosis Studies. All animal experiments were performed in accordance with the Animal Care and Use Committee, University of Ottawa, Canada. Eight week old Apo E^{-/-} mice (total n=17 mice) were fed an adjusted calories diet (21% fat; 0.2% cholesterol, Harlan Teklad) for 4 weeks to induce atherosclerotic progression. Mice were then injected with time-release placebo or Nec-1s tablets (2 mg/kg/day), which were synthesized by Innovative Research of America, USA. Mice were euthanized after 6 weeks of treatment, perfused with saline and aortic roots were embedded in OCT medium and frozen. Aortic roots were sectioned (10 μm) and stained with hematoxylin and eosin for lesion area and necrotic core quantification, and a minimum of 10 sections per animal were measured across the length of the entire aortic root. For en face aortic lesion quantifications, aortas were dissected removing all branching vessels down to the femoral bifurcation and then sliced ventrally and images of aortas were digitally captured using ImagePro™. Lesion areas within the entire length of the aorta were quantified using Image J™ and expressed relative to the total aortic surface area. Immunohistochemistry for vascular smooth muscle cells was performed using a primary antibody for a-smooth muscle actin (monoclonal, Sigma) conjugated to alkaline phosphatase and visualized using SigmaFAST Red (Sigma). Statistical significance between the two groups were analyzed using Students' t-test (p<0.05).

Radiochemistry and ex vivo autoradiography study with ApoE^{-/-} mice. The tracer ¹²³I-Nec-1 (7-¹²³I-Nec-1) was synthesized by a Cu(I) catalyzed direct halogen exchange reaction with the precursor 7-Br-Nec-1 and Na¹²³I. Eight week-old female apolipoprotein E knockout (Apo E^{-/-}) mice were purchased from Charles River Laboratories and fed either a chow or western diet (TD.10885, Harlan Laboratories) for 2 months (total n=6 mice). 7-¹²³I-O-Nec-1 (37.0-55.5 MBq) was administrated intravenously into the mice under anesthetic. After 2 hr, the mice were euthanized and perfused with PBS followed by 10% formalin via left ventricle cannulation. Aorta was dissected from heart at the base using a dissecting microscope following removal of surrounding fat and connective tissue. En face specimens were immediately exposed to super resolution phosphor screens in an autoradiography cassette. After overnight exposure at room temperature, the screens were scanned with a Cyclone Phosphor Imager (Perkin Elmer, Downers Grove, Ill.). Images were analyzed using OptiQuant™ 5.0 software. The regions of interest (ROI) were drawn around the lesions on the aortic arch, and the counts in Digital Light Unit (DLU) and surface areas in mm² were measured. The DLU was converted to activity in μCi using a set of calibration standards with known activities, which were exposed and scanned on the same screen used for the aorta samples, as previously described (Zhao et al, 2007; Matter et al., 2006). The percentage injected dose (% ID) was calculated from dividing the activity (pCi) of the lesion by the injected activity. Activity density in % ID/m² was calculated and normalized by animal body weight to get % ID×kg/m². The areas of the lesions and the whole aortic arch in mm² were also recorded using the OptiQuant™ 5.0 software, and the percentage of lesion within the area of the aorta vessel wall was calculated.

Human atherosclerotic lesion analysis. Human arterial samples were obtained from the Biobank of Karolinska Endarterectomy (BiKE) at the Centre for Molecular Medicine, Karolinska Institute previously described (Razuvaev et al, 2011; Perisic et al., 2013). In brief, control normal arteries (undiseased macroscopically atherosclerosis free-arteries, iliac and one aorta) were obtained from organ donors without any current or history of cardiovascular disease. Atherosclerotic plaques were obtained from patients undergoing surgery for stable or unstable carotid stenosis. All samples were collected with informed consent and the study was approved by the Ethical Committee of Northern Stockholm. Plaque tissues were ruptured with a Tissue Rupturer/Homogenizer (Omni Inc) and RNA isolated. Gene expression profiles were obtained from n=10 control arteries and n=127 atherosclerotic plaque samples and statistical analysis was performed using Students' t-test (p<0.05).

Statistics. Data shown is either mean±SD of a single representative experiment or mean±SEM of at least 3 independent experiments performed in triplicates, and is indicated in the corresponding figure legends. Comparison between control and treatment or treatment and treatment plus inhibition was made using Student's t-test (p≦0.05) or comparison between groups by one-way ANOVA (p≦0.05) or two-way ANOVA (p≦0.05) using Prism GraphPad™.

### Results

A summary of this Example is provided here, followed by more specific detail. Bone-marrow derived macrophages (BMDMs) treated with oxLDL had increased cell death, which was inhibited with necrostatin-1 (Nec-1), a specific inhibitor of necroptosis. Macrophages treated with oxLDL had increased expression of necroptotic genes RIP1, RIP3 and MLKL, and phosphorylation of RIP3—a critical step in the execution of necroptosis. Further, the combined treatment of BMDMs with oxLDL and DAMPs amplified necroptotic cell death, indicating that additional inflammatory stimuli present in the lesion could act synergistically to promote necroptosis. Further, analysis of unstable human atherosclerotic plaques show that they have a marked increase in RIP3 and MLKL gene expression relative to healthy controls. To test if necroptosis promotes atherosclerotic lesion formation, ApoE−/− mice were fed a western diet for 4 weeks to induce atherosclerosis and subsequently treated with Nec-1 inhibitor for an additional 6 weeks. Morphometric analysis of atherosclerotic lesions showed that Nec-1 treated mice had reduced lesion area in the aortic sinus and aorta compared to placebo-treated mice (p<0.05 and p<0.0001, respectively). Further, these mice had a marked reduction in total necrotic area within the aortic sinus (62.6% reduction, p<0.01), illustrating that Nec-1 therapy may be used to treat for cardiovascular diseases such as atherosclerosis. A ¹²³I-radiolabelled Nec-1 tracer for pSPECT imaging is described and shows that ¹²³INec-1 specifically localizes to advanced atherosclerotic lesion areas in ApoE−/− mice and thus can be used as a diagnostic for unstable atherosclerotic disease.

OxLDL induces necroptosis in macrophages via RIP3. In order to test whether oxLDL is sufficient to induce necroptotic cell death, bone marrow derived macrophages (BMDMs) was incubated with extensively oxidized LDL for 24 h and the degree of cell death was measured.

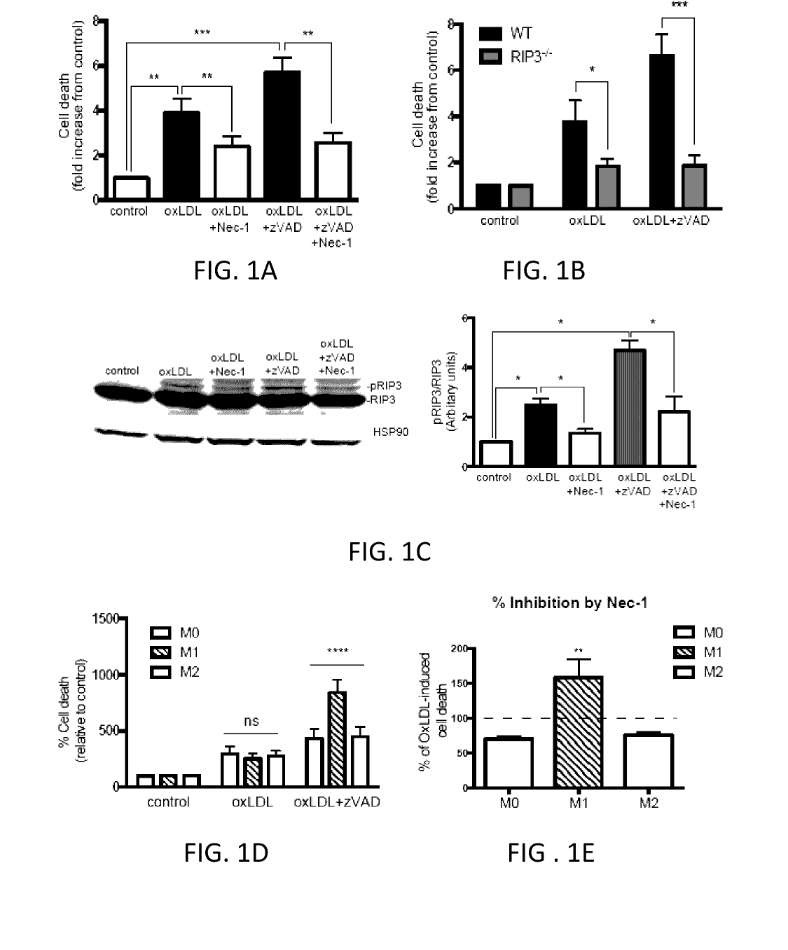

FIG. 1A to FIG. 1E show that oxidized LDL induces necroptotic cell death in macrophages. In FIG. 1A, bone-marrow-derived macrophages (BMDMs) were treated with 100 μg/mL oxLDL±zVAD.fmk±Nec-1 for 24 h and LDH release in the media was measured. Data represents mean±SEM of 5 independent experiments. In FIG. 1B, cell death in response to oxLDL±zVAD.fmk in BMDMs from WT and Rip3^{-/-} mice is shown. In FIG. 1C, Western blot analysis of RIP3 after treatment with oxLDL±zVAD.fmk±Nec-1 for 8 hours is shown. Band shift indicates phospho-RIP3. The right panel shows quantification of phospho-RIP3 normalized to total RIP3 by densitometry. Data represents mean±SEM of 3 independent experiments. In FIG. 1D, cell death in response (as above) in resting (M0), M1- and M2-polarized macrophages treated with oxLDL±zVAD.fmk for 24 h is shown. In FIG. 1E, Inhibition of oxLDL-induced cell death by Nec-1 in resting (M0), M1- and M2-polarized macrophages treated with oxLDL for 24 h is shown.

All data represents mean±SEM of at least 3 independent experiments. *p≦0.05, **p<0.01, ***p<0.001 by one-way ANOVA (FIG. 1A-FIG. 1C) or two-way ANOVA (FIG. 1D-FIG. 1E).

Compared to unstimulated cells, oxLDL treatment resulted in a ˜4-fold induction of cell death in macrophages and this was significantly inhibited by co-treatment with Nec-1, a specific inhibitor of necroptosis (3.9±0.6-fold vs. 2.3±0.4-fold, respectively; p≦0.01; FIG. 1A). Moreover, inhibition of apoptotic cell death with the pan caspase inhibitor zVAD.fmk significantly enhanced cell death in response to oxLDL, which was similarly inhibited by Nec-1 (5.7±0.7 -fold, p≦0.001; FIG. 1A).

To test whether the induction of cell death is dependent upon RIP3 function, the induction of cell death by oxLDL and oxLDL+zVAD.fmk was measured in both wild-type macrophages and macrophages deficient in RIP3 (Rip3^{-/-}) and observed that Rip3^{-/-} macrophages are resistant to cell death in response to both oxLDL and oxLDL+zVAD.fmk (FIG. 1B). The degree of phosphorylation of RIP3 was then measured in response to oxLDL, as phospho-RIP3 is required for the execution of necroptosis. OxLDL significantly induced RIP3 phoshorylation in macrophages, which was dampened by Nec-1 (FIG. 1C). Taken together, these data demonstrate that oxLDL, a potent atherogenic ligand, can independently induce cells to undergo necroptosis via activation of RIP3 and MLKL expression, and this is exacerbated by inhibition of caspases with zVAD.fmk.

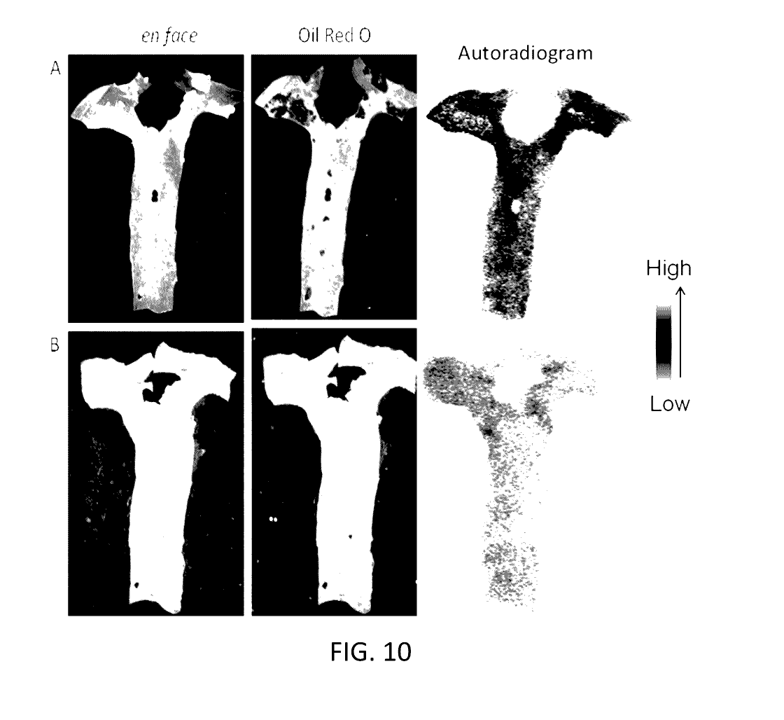

Within the atherosclerotic plaque, several sub-types of macrophages can be found, and a progressing atherosclerotic lesion is predominantly composed of pro-inflammatory M1 macrophages whereas the stable or regression plaque is composed of anti-inflammatory M2 macrophages (Peled et al., 2014). The degree of cell death in response to oxLDL in M1 and M2 polarized macrophages was tested. It was found that macrophages polarized to an M1 phenotype have a higher propensity towards cell death in response to oxLDL+zVAD.fmk, while oxLDL alone induces cell death to a similar degree in resting, M1- and M2-polarized macrophages (FIG. 10). While Nec-1 was able to inhibit cell death as expected in resting macrophages, it similarly inhibited oxLDL-induced death in M2 polarized macrophages but was unable to inhibit death in M1-polarized cells (FIG. 1E). Therefore, these data indicate that macrophages that are polarized towards a pro-inflammatory phenotype may have increased susceptibility to cell death induced by atherogenic LDL, and if apoptosis is inhibited or overwhelmed, these cells die via the necroptotic pathway.

DAMPs exacerbate oxLDL-induced necroptosis. Within the atherosclerotic milieu, various stimuli are present in addition to oxLDL that can promote the inflammatory response and propagate lesion formation. These include cytokines, chemokines, reactive oxygen species, cholesterol crystals and other modified self-ligands, and together are termed damage-associated molecular patterns (DAMPs). DAMPs are released from cells during the process of necroptosis, where uncontrolled leakage of intracellular components occurs after membrane integrity is lost. It was thus tested whether DAMPs released in response to necroptosis exacerbate oxLDL-induced cell death.

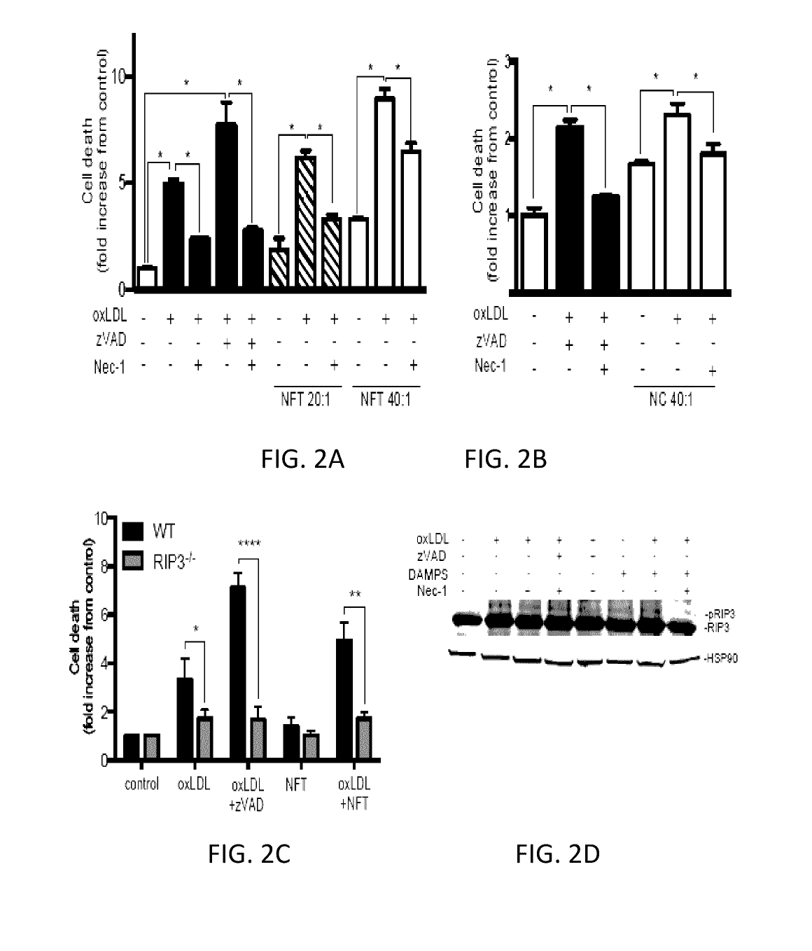

FIG. 2A to FIG. 2D show that a combination of oxLDL and DAMPs increases macrophage necroptosis. In FIG. 2A BMDMs were subjected to freeze thaw to generate necrotic DAMPs (NFT) which were added to cells with or without oxLDL±Nec-1 for 24 h and cell death was measured. In FIG. 2B BMDMs were treated with necroptotic inducers LPS+zVAD.fmk for 24 h to generate necroptotic cell DAMPs (NC), which were added to cells with or without oxLDL±Nec-1 for 24 h and cell death measured. In FIG. 2C cell death in response to oxLDL±zVAD.fmk±necrotic freeze thaw (NFT) DAMPs in BMDMs from WT and Rip3^{-/-} mice was evaluated. FIG. 2D shows Western blot analysis of RIP3 after treatment with oxLDL±zVAD.fmk ±Nec-1 with or without NFT DAMPs for 8 hours. Band shift indicates phospho-RIP3. Data are representative of at least 3 independent experiments.

Indeed, co-incubation of DAM Ps generated by either mechanical (i.e. freeze-thaw) or pharmacological (i.e. LPS+zVAD.fmk) means together with oxLDL significantly induced cell death in macrophages, which could be inhibited by Nec-1 (FIG. 2A and FIG. 2B; p<0.05). Like what is observed with oxLDL alone, cell death in response to DAMPs is dependent upon RIP3, as Rip3^{-/-} macrophages are resistant to cell death by oxLDL+DAMPs (FIG. 2C). Activation of RIP3 by phosphorylation is increased when macrophages are co-incubated with oxLDL and DAMPs, and can be inhibited by Nec-1 (FIG. 2D). These data indicate that in addition to oxLDL, DAMPs released from dying cells exacerbate necroptotic cell death and is dependent on RIP3 activation, indicating that the multiple inflammatory ligands present in the atherosclerotic plaque may act concomitantly to promote necroptosis.

Oxidized LDL induces expression of RIP3 and MLKL. OxLDL is an inflammatory ligand that induces the activation of pro-inflammatory signaling events, eliciting cytokine and chemokine production. To examine the mechanisms underlying the induction of necroptosis by oxLDL, expression was measured of genes involved in the necroptotic pathway in macrophages stimulated with this atherogenic ligand.

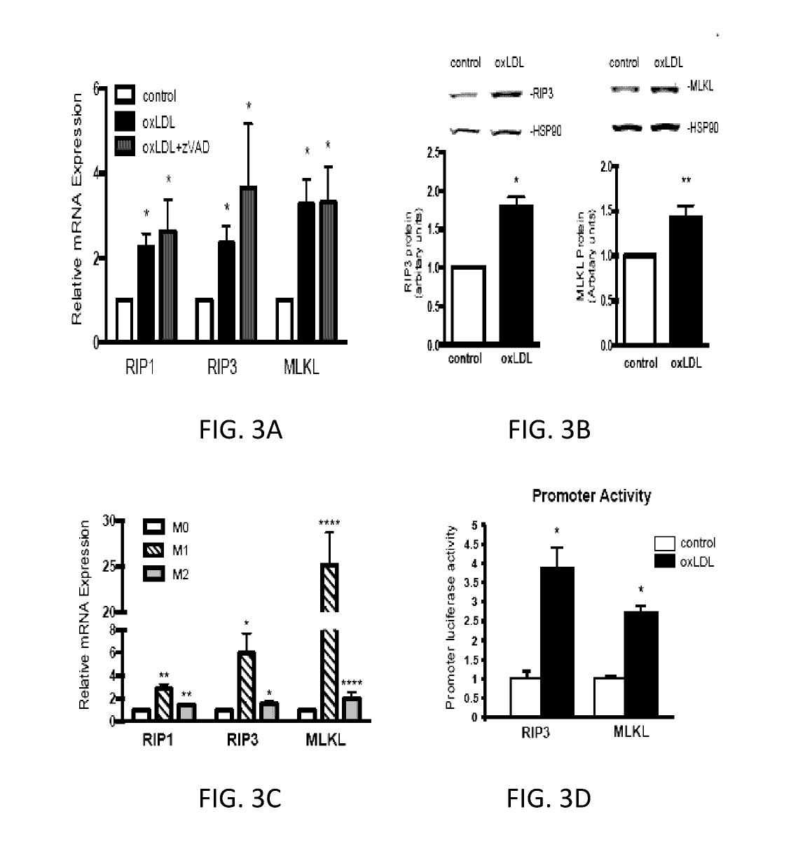

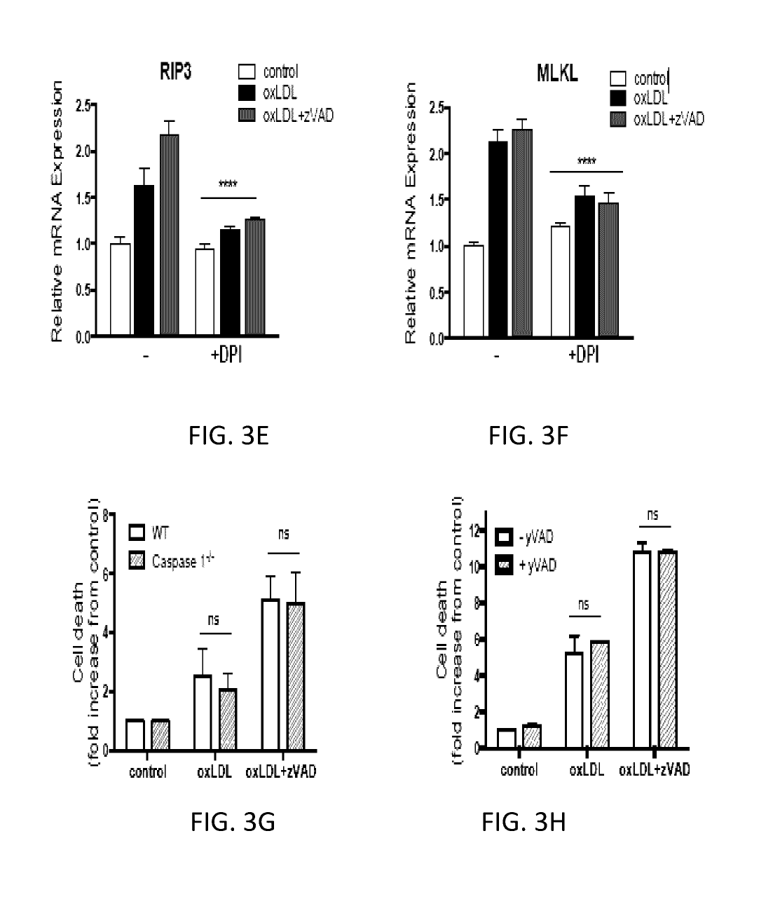

FIG. 3A to FIG. 3H show that OxLDL induces the expression of RIP3 and MLKL. In FIG. 3A, BMDMs treated with 100 μg/ml oxLDL±zVAD.fmk for 24 h were analyzed for gene expression of RIP1, RIP3 and MLKL by qPCR. FIG. 3B shows Western blot analysis of RIP3 and MLKL expression in BMDMs treated with oxLDL. Quantification below of at least 3 independent experiments. FIG. 3C shows gene expression of RIP1, RIP3 and MLKL assessed by qPCR in BMDMs at rest (MO) or polarized to an M1 or M2 phenotype.

In FIG. 3D, RAW macrophages were transfected with RIP3-promoter or MLKL-promoter luciferase constructs and treated for 24 h with oxLDL before luciferase expression was examined and expressed as promoter-luciferase activation normalized to control (no treatment). In FIG. 3E and FIG. 3F, RIP3 or MLKL mRNA expression, respectively, is measured by qPCR in BMDMs treated with oxLDL±zVAD.fmk, in the presence or absence of pre-treatment with 50 μM DPI (Reactive Oxygen Species inhibitor). In FIG. 3G cell death in BMDMs from WT and Casp1^{-/-} mice is shown in response to oxLDL±zVAD.fmk for 24 h. In FIG. 3H cell death in response to oxLDL±zVAD in BMDMs treated with or without the caspase-1 inhibitor zYVAD.fmk (20 uM) is shown. In these figures, *p≦0.05, **p<0.01, ***p<0.001, p<0.0001 by one-way ANOVA.

Treatment of macrophages with oxLDL induced the expression of RIP3 and MLKL at both the mRNA (FIG. 3A) and protein level (FIG. 3B). Interestingly, the expression of RIP3 and MLKL are significantly higher in M1 polarized macrophages compared to resting or M2 macrophages (FIG. 3C).

To assess whether this increase in gene expression was due to direct activation of the promoter by oxLDL, a synthetic construct with the promoter region of either RIP3 or MLKL upstream of a luciferase reporter was used. In RAW macrophages transfected with a RIP3-promoter or MLKL-promoter, oxLDL significantly induced the activation of the luciferase reporter, indicative of an activation of the promoter regions of RIP3 and MLKL by oxLDL (FIG. 3D). Given the ability of oxLDL to induce the production of reactive oxygen species (ROS), it was tested whether ROS production was critical for the induction of RIP3 and MLKL expression.

Pre-treatment of macrophages with the ROS inhibitor diphenyleneiodonium (DPI) inhibited the induction of both RIP3 and MLKL mRNA expression by oxLDL and oxLDL+zVAD.fmk (FIG. 3E and FIG. 3F). These results indicate that oxLDL induces the expression of RIP3 and MLKL at the mRNA and protein levels likely as a direct result of activation of the promoter regions of these genes.

oxLDL is an inducer of the NLRP3 inflammasome, and can induce the expression and secretion of IL-1β. It was therefore evaluated whether the inflammasome was required for the induction of necroptosis by oxLDL. Macrophages from either wild-type or caspase-1-deficient mice (Casp1^{-/-}) had equivalent levels of cell death upon treatment with oxLDL (FIG. 3G). Similarly, co-treatment of macrophages with the caspase-1 inhibitor zYVAD.fmk did not reduce or prevent cell death in response to oxLDL (FIG. 3H). These data indicate that necroptotic cell death in response to oxLDL does not depend on the induction of caspase-1, unlike what is observed for IL-1β secretion.

Blocking necroptosis in established lesions reduces plaque size and markers of vulnerability. It was investigated whether inhibition of necroptosis via Nec-1 could reduce markers of lesion vulnerability in ApoE−/− with established atherosclerosis.

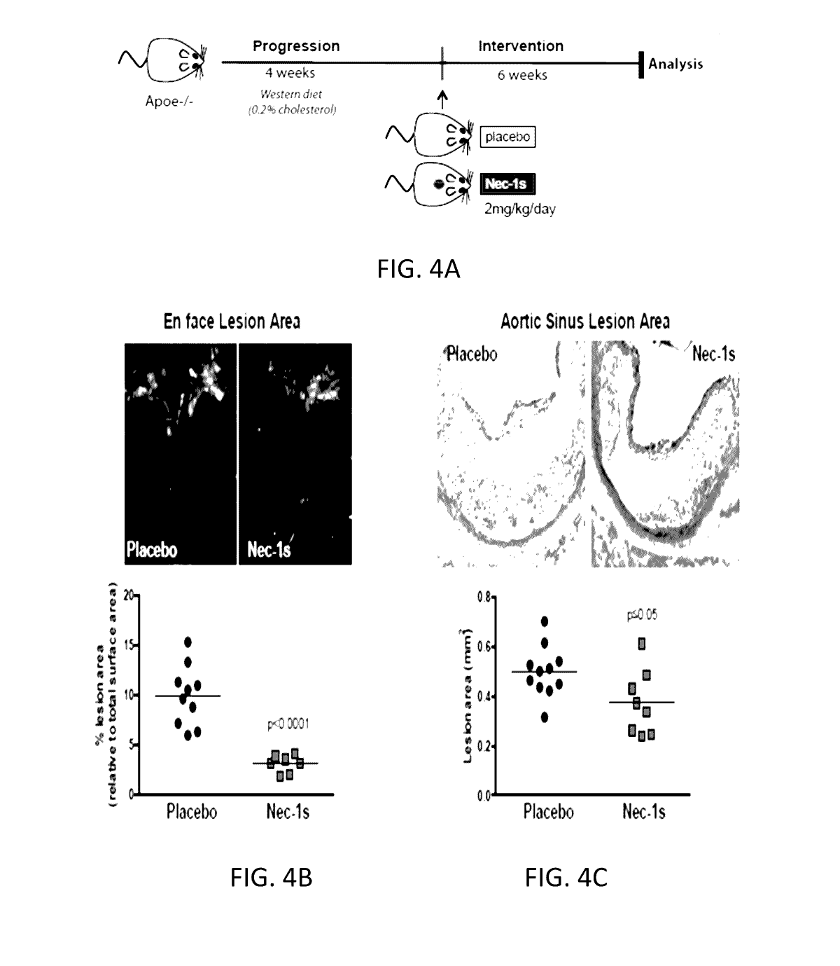

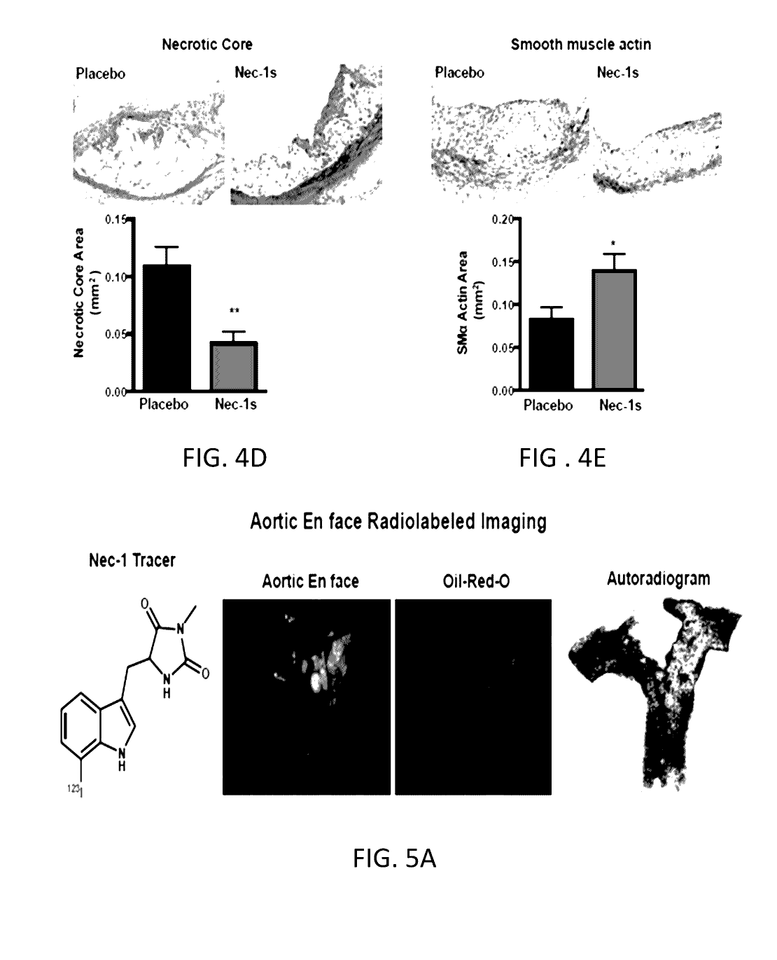

FIG. 4A to FIG. 4E illustrate that Nec-1 therapy decreases atherosclerotic lesion progression and markers of instability in ApoE^{-/-} mice. In FIG. 4A ApoE^{-/-} mice were fed a Western diet for 6 weeks prior to implantation of time-release pellets containing placebo or Nec-1s (2 mg/kg/day). After 4 additional weeks of Western diet feeding, mice were harvested for morphometric analysis of atherosclerosis. In FIG. 4B En face lesion area was measured in placebo and Nec-1s treated mice and is represented as lesion area as a % of total aorta area. In FIG. 4C lesion area in the aortic sinus in placebo and Nec-1s treated mice is represented as total area in μm². In FIG. 4D and In FIG. 4E, respectively, necrotic core area and smooth muscle actin staining within aortic sinus lesions were quantified with Image J. Representative images per group are included. *p≦1.05, **p<0.01 by Student t-test.

After feeding a Western diet for 4 weeks, ApoE−/− mice were implanted with a time-release pellet containing either Nec-1 or placebo, and lesions were allowed to progress for a further 6 weeks (FIG. 4A). Quantification of en face lesion area in ascending and descending aortas revealed that Nec-1 treatment significantly reduced lesion burden compared to placebo treated mice by 68% (9.9±0.9% placebo versus 3.1±0.3% Nec-1; p≦1.0001; FIG. 4B). In the aortic root, lesion area was reduced by 27% compared to controls (0.50±0.03 mm² placebo versus 0.37±0.05 mm² Nec-1; p≦1.05; FIG. 4C). While ApoE−/− mice do not typically develop plaque rupture, they do develop markers of lesion vulnerability; that is, large necrotic cores and reduced smooth muscle cell fibrous cap content. Evaluation of lesion characteristics demonstrate that Nec-1 reduced lesion necrotic core area by 62% compared to placebo (p≦1.01; FIG. 4D) and increased smooth muscle actin area within the aortic sinus by 70% (p≦1.05, FIG. 4E). These results demonstrate that in mice with established early atherosclerotic lesions, intervention with a pharmacological inhibitor of necroptosis can prevent further lesion progression and increase markers of plaque stability (i.e. decreased necrotic core), indicating that necroptosis underlies both the development and vulnerability of atherosclerotic plaques and is a therapeutic target.

Imaging of atherosclerosis in vivo using radiolabeled Nec-1. Given that necroptosis is contributing to the formation of atherosclerotic lesions in mice, the necroptotic pathway may be useful as a biomarker for molecular imaging to visualize atherosclerotic lesion development in vivo. An ¹²³I-labeled Nec-1 compound was developed, and its localization to aortic lesions was evaluated in ApoE−/− mice. Radiolabeled Nec-1 (¹²³I-Nec-1) was injected into ApoE−/− mice with established lesions and 2 hours later aortas were harvested and analyzed for radiotracer uptake.

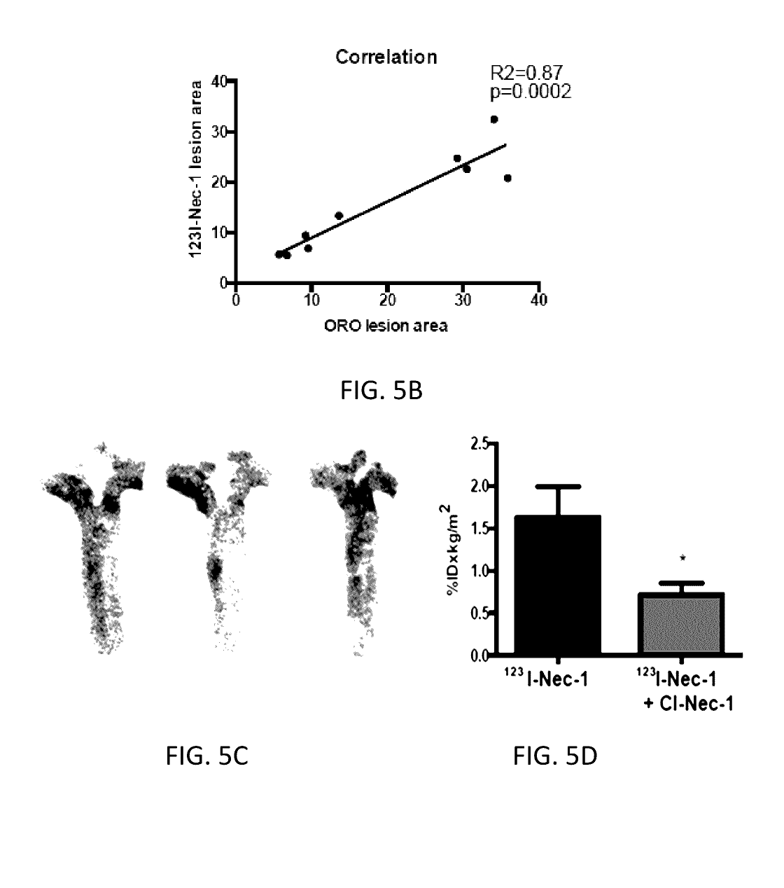

FIG. 5A to FIG. 5C show that radiolabeled Nec-1 can be utilized to visualize atherosclerotic plaques in ApoE^{-/-} mice. In FIG. 5A the left side shows the chemical structure of the 7-¹²³I-Nec-1 tracer. Central panels are representative images of aortic en face (no stain and oil red-O [ORO] stain) and the right side panel shows a representative autoradiogram from mice injected with ¹²³I-labelled Nec-1 tracer. FIG. 5B shows the correlation of lesional uptake of ORO compared to ¹²³I-Nec-1 (n=9). In FIG. 5C mice were injected with non-radioactive Cl-Nec-1 compound 1.5 h prior to being injected with radiolabelled ¹²³I-Nec-1 tracer, subjected to autoradiography (images shown) and lesional uptake quantification (%IDx kg/m³) is show in FIG. 5D (n=3 mice per group, p≦1.05 Cl-Nec-1 vs. ¹²³I-Nec-1 uptake by t-test).

Autoradiography imaging revealed that ¹²³I-Nec-1 was primarily localized to areas of plaque accumulation in the en face aortic arch (FIG. 5A). When compared to oil red O (ORO) uptake within the aorta, which stains lipid-rich lesion areas, there was a significant correlation between lesion area measured by ¹²³I-Nec-1 and ORO (FIG. 5B). Pre-administration of a cold non-radioactive Nec-1 compound significantly blocked aortic lesional uptake of ¹²³I-Nec-1 (FIG. 5C and 5D), indicating that radiolabeled Nec-1 uptake is specific. Thus, a novel molecular imaging tool is developed using a small molecular inhibitor of necroptosis as a radiotracer, which can be utilized to visualize atherosclerotic lesion areas.

Necroptotic pathway is upregulated in unstable atherosclerosis in humans. To assess whether the necroptotic pathway is upregulated during atherogenesis in humans, the expression of RIP3 and MLKL was analysed in carotid plaques from patients with atherosclerosis and in disease-free control arteries using methods described previously (Razuvaev et al., 2011; Perisic et al., 2013).

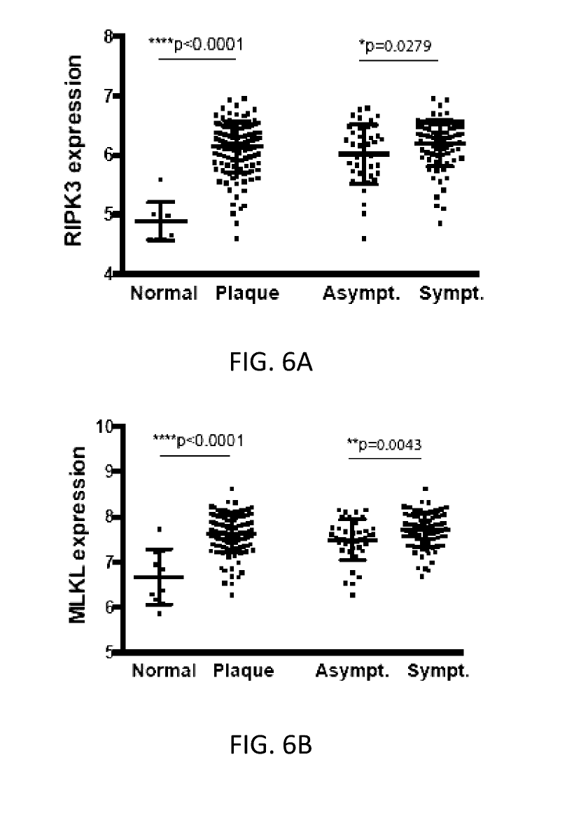

FIG. 6A and FIG. 6B show up-regulation of necroptotic genes in unstable atherosclerosis. mRNA expression of RIP3 (FIG. 6A) and MLKL (FIG. 6B) in carotid endarterectomies (plaque) from asymptomatic (asymptomatic) or symptomatic patients (symptomatic), or macroscopically disease-free control arteries (normal), obtained from the Biobank of Karolinska Endarterectomies (BiKE). *p≦1.05, **p<0.01, ****p<0.0001 by Student t-test.

There was a significant upregulation in expression of both RIP3 and MLKL in atherosclerotic plaques compared to normal arteries (p≦1.0001; FIG. 6A). Necroptosis was found to underlie lesion vulnerability. Thus, it was subsequently examined in this Example whether expression of necroptotic genes may be further increased in unstable versus stable atherosclerotic plaques. Gene expression analysis of carotid plaques from individuals with symptomatic carotid disease (i.e. transient ischemic attack, minor stroke and/or amaurosis fugax) revealed a significant elevation of both RIP3 and MLKL gene expression compared to plaques from asymptomatic individuals (p≦1.05 and p≦1.01, respectively, FIG. 6B). Therefore, as was observed in vitro in response to atherogenic oxLDL, necroptotic genes RIP3 and MLKL are significantly elevated in human atherosclerotic lesions and are associated with plaque instability, indicating that necroptosis is active within vulnerable lesions.

### Discussion

In this Example, a new pathway underlying atherosclerotic plaque vulnerability is identified, that is induced by atherogenic ligands and that can be targeted for therapeutic and diagnostic interventions. OxLDL, an inflammatory form of LDL that is abundant within developing atheroma, is sufficient to induce necroptotic cell death in macrophages, but is enhanced when combined with the pan-caspase inhibitor zVAD-fmk. Apoptotic cell death is triggered when caspase-8 is active, resulting in cleavage and inactivation of RIP1 and RIP3. Conversely, when caspase-8 is rendered inactive the RIP1/RIP3 complex forms and promotes downstream activation of necroptosis, including activation of MLKL, mitochondrial damage and membrane rupture. Although oxLDL is known to induce apoptosis, this example is the first to find that oxLDL can directly induce necroptotic cell death in the absence of synthetic caspase inhibition. This Example also illustrates that necroptosis can be targeted with therapeutic inhibition in a mouse model of atherosclerosis, which reduces lesion size and markers of vulnerability. Importantly, it was demonstrate that necroptosis is activated within human atherosclerotic plaques, and that increased RIP3 and MLKL gene expression are associated with unstable vascular disease. Overall, this Example outlines a novel mechanism that underlies atherosclerotic plaque vulnerability that is driven by macrophage inflammatory cell death.

The expression of RIP3 is tightly correlated with the degree of necroptosis, and induction of RIP3 expression can overcome inhibition by caspase-8 and thus trigger necroptotic cell death. It is shown in this Example that oxLDL induces the expression of necroptotic genes RIP1, RIP3 and MLKL, and results in the upregulation and phosphorylation of RIP3 protein- a requirement for the assembly of the RIP1-RIP3 complex and subsequent necroptosis. Similar to NLRP3, whose mRNA induction by oxLDL is considered a necessary priming step for inflammasome activation, it is shown in this Example that oxLDL induces mRNA and protein expression of RIP3 and is sufficient to activate necroptosis. OxLDL is known to induce many downstream signaling events, including the generation of ROS, which can subsequently activate transcription factors to induce redox-sensitive gene expression. This Example finds that inhibition of ROS production interferes with the induction of RIP3 and MLKL expression by oxLDL, which parallels the findings of Bauernfeind et al (2011) that ROS is required for the induction of NIrp3 expression prior to inflammasome activation. Moreover, macrophages from Rip3^{-/-} mice do not undergo cell death in response to oxLDL, confirming that the expression of RIP3 is required for oxLDL-mediated induction of necroptosis. This Example is the first evidence that oxLDL triggers necroptosis independently from caspase-8 inhibition by upregulation of necroptotic cell genes RIP3 and MLKL, and is exacerbated in pro-inflammatory macrophages. This Example illustrates that the induction of necroptosis by oxLDL is independent of inflammasome activation, as cells deficient in caspase-1 or treated with caspase-1 inhibitors undergo necroptotic cell death in response to oxLDL to the same degree as wild-type or untreated cells. Interestingly, pro-inflammatory M1 macrophages have a higher expression of RIP3 and MLKL than resting or M2 macrophages, yet these cells are insensitive to Nec-1, indicating that elevated RIP3 and MLKL may push the cells beyond the ability of Nec-1 to recover necroptotic cell death. Increased RIP3 and MLKL gene expression are positively associated with unstable atherosclerotic lesions compared to both stable lesions and healthy arteries. Collectively these data demonstrate that oxLDL induces the expression of the necroptotic pathway via induction of gene expression to promote necroptotic cell death, and this necroptotic pathway is significantly elevated in plaques from patients with atherosclerotic vascular disease.

Necroptosis results in the uncontrolled release of cellular antigens into the extracellular space, where this cellular material can then serve as DAM Ps to initiate an innate immune response via pattern recognition receptors. In addition to oxLDL, the atheroma milieu is rich in other sources of DAMPs, such as other oxidized lipids, inflammatory cytokines, heat shock proteins and mitochondrial DNA. It was investigated whether DAM Ps generated from necrotic cells exacerbate cell death by necroptosis induced by oxLDL. Indeed, treatment of macrophages with oxLDL in combination with DAMPs generated from necrotic cells induced more cell death than treatment with oxLDL alone, and was inhibited by Nec-1. Similarly, macrophages from Rip3−/− mice did not undergo cell death in response to stimulation of oxLDL+DAMPs and treatment of macrophages with DAMPs induces phosphorylation of RIP3, indicating that necroptosis is indeed dependent upon RIP3 activation. These data indicate that within the atherosclerotic plaque, there is a feed-forward loop of necroptosis activation first by oxLDL, then by the DAMPs released in response to necroptotic cell death that may exacerbate the extensive necrotic core found in advanced lesions.

Throughout virtually all stages of atherosclerosis, cell death is active and contributes to plaque progression, from early foam lesion formation to the advancement to unstable rupture-prone plaques. In the early stages of lesion initiation, apoptosis is induced by oxidized lipids in the subendothelial space and apoptotic cells are rapidly cleared by phagocytes by efferocytosis and thus apoptosis in the early stages of atherosclerosis appears to be protective. In later stages it is believed that efferocytosis is impaired, leading to the accumulation of apoptotic cell debris and the induction of inflammation, contributing to lesion destabilization. So long as oxLDL persists within the plaque, macrophages undergo necroptotic (as well as apoptotic) cell death, releasing DAMPs into the extracellular space. As necrotic cells are not efficiently cleared by efferocytosis, this results in further activation of inflammation and necroptotic cell death, and in advanced lesions, necroptosis may dominate. The accumulation of DAMPs induced by atherogenic ligands further exacerbates cell death and may not be cleared effectively by efferocytosis. Indeed, this is in line with the concept that in advanced human atherosclerotic lesions, cells morphologically resembling necrosis are found to a greater extent than those undergoing apoptosis.

It was found that intervening with the pharmacological inhibitor of necroptosis, Nec-1, in mice with established early atherosclerosis reduces both aortic lesion area and markers of plaque instability (i.e. necrotic core), demonstrating that necroptotic cell death is ongoing within the atherosclerotic plaque and directly contributes to plaque progression and ultimately instability. These results agree with those found in a related model of atherosclerotic mice (LdIr−/−) crossed with mice lacking Rip3, who developed smaller lesions than wild-type mice with intact Rip3 expression (Lin et al., 2013). These authors showed that oxLDL combined with the pan caspase inhibitor zVAD.fmk induces necroptotic cell death that is absent in Rip3−/− mice. It is shown herein that oxLDL does not require chemical inhibition of apoptosis but rather can induce necroptotic death independently.

This Example provides the first finding that endogenous atherogenic ligands such as oxLDL and DAMPs can induce macrophage necroptosis without the use of chemical inhibitors of apoptosis, indicating that this pathway is indeed activated by physiological endogenous signals. Furthermore, it is demonstrated in this Example that intervention with Nec-1 dramatically reduces lesion and necrotic core area, which offers therapeutic excitement for the treatment of established atherosclerosis, which goes beyond the prevention of atherosclerosis by genetic deletion of Rip3. While Nec-1 was first described as a specific inhibitor of RIP1/RIP3 interaction and hence activation of necroptosis, some of the activity of Nec-1 may render the RIP1-dependent apoptotic pathway inactive. Nec-1 may serve, in vivo to inhibit both apoptotic—as well as necroptotic-cell death, which ultimately both contribute to lesion instability at later stages of atherosclerosis. This Example is the first report of Nec-1 being used in a long-term therapeutic application, and provides excitement for the potential of next-generation necroptosis inhibitors with improved specificity and stability for the treatment of chronic inflammatory diseases like atherosclerosis.

Current clinical practice relies on invasive angiography to visualize plaques in patients with atherosclerosis, which provides limited detail of plaque size but no insight into plaque inflammation or vulnerability. Imaging by positron emission tomography (PET) or single photon emission computed tomography (SPECT) provides the advantage of being non-invasive, with the ability of detecting specific molecular processes that provide insight into the pathology of the plaque when specific molecular tracers are used. ¹⁸F-fluorodeoxyglucose (FDG), a radiolabled glucose analog, is the most widely-used nuclear tracer to detect inflammation within atherosclerotic plaques. Inflammatory cells like macrophages have a high metabolic demand for glucose, and therefore take up FDG at a higher rate than other non-inflammatory cells, making FDG a surrogate of inflammation within the plaque.

FDG-PET has supported the concept that molecular imaging of dominant pathways within the atherosclerotic plaque correlates with lesion vulnerability and thus may be of added value when assessing overall cardiovascular risk. However, there remains a need to develop novel radiotracers that can detect coronary artery inflammation without the complication of myocardial uptake, and with less sensitivity to metabolic characteristics of the patient (i.e. high fasting blood glucose). Given the finding that the necroptosis inhibitor Nec-1 can reduce lesion size in a mouse model of atherosclerosis, it was investigated whether this same necroptosis inhibitor could be used as a tool to visualize atherosclerotic lesions in vivo. A radiolabeled iodine moiety was added on to the Nec-1 molecule, which can be detected using autoradiographic imaging. By co-registering autoradiography images together with en face images, it is shown that ¹²³I-Nec-1 co-localizes to the atherosclerotic plaques in ApoE−/− mice. The lesional uptake correlated very strongly with that of traditional ORO uptake, indicating that Nec-1 may be a novel method to detect the presence of atherosclerotic lesions in vivo. Although the uptake of the Nec-1 radiotracer was only measured ex vivo in the current study, similar radiotracers can be developed and labeled with PET radionuclides and allow Nec-1 to be used in the future for non-invasive PET imaging in animal models, with the ultimate goal of detecting atherosclerotic lesions in patients with disease.

In summary, this Example describes a mechanism whereby oxLDL and other DAMPs activate necroptotic cell death in macrophages, and this can be inhibited upon treatment with Nec-1. These studies are the first to show that an atherogenic ligand can directly induce the expression of necroptotic genes via activation of the promoter, which may be downstream of reactive oxygen species production. In a mouse model of atherosclerosis, intervention with Nec-1 prevented further lesion progression and reduced markers of lesional vulnerability, underscoring the importance of necroptosis in atherosclerotic lesion progression and the development of the necrotic core. Targeting necroptosis using a radiolabeled Nec-1 tracer enabled the detection of atherosclerosis by nuclear imaging that could be used to measure atherosclerotic lesion burden in mice. Importantly, in patients with carotid atherosclerosis, RIP3 and MLKL expression levels are markedly increased, and are further elevated in unstable versus stable lesions. The Example affirms the use of necrostatins, and the targeting of necroptosis, as a diagnostic tool, using either nuclear imaging and/or biomarker expression, as well as for the development of therapies that specifically inactivate the necroptotic pathway for the treatment and management of clinically vulnerable atherosclerosis.

Example 2

### An I-123 Labeled Necrostatin-1 Derivative as a Novel Potential SPECT Imaging Agent Targeting Necroptosis.

### Overview

Cell death plays an important role in both normal and pathophysiology, and was thought to occur by either spontaneous rupture of cell membranes (i.e. necrosis) or specific programmed multi-step pathway (i.e. apoptosis). Necroptosis, or programmed cell necrosis, is tightly regulated by RIP kinases and can be specifically inhibited by necrostatin-1 (Nec-1). Radiolabeled necrostatin-1 is described herein, which can be utilized as an imaging agent to target and visualise necroptosis in vivo for diagnostic purposes. In this Example, 7-¹²³I-O-Nec-1 was synthesized by a Cu(I) catalyzed direct halogen exchange reaction with the precursor 7-Br-O-Nec-1. Its stability was tested in rat serum at 37° C. for 24 hr. Biodistribution studies were conducted in wild-type Sprague-Dawley rats at 2 hr post injection (p.i.) and C57BL/6 mice at 1 hr and 2 hr p.i. To validate the utility of 7-¹²³I-O-Nec-1 in a pathological model where necrosis is known to occur, 7-₁₂₃ I-O-Nec-1 was injected into ApoE^{-/-} mice with atherosclerotic lesions and racer uptake was evaluated by ex vivo en face autoradiography imaging methodology and Oil Red O staining. In this Example, it is shown that 7-¹²³I-O-Nec-1 was successfully synthesized and purified by RP-HPLC to achieve >95% radiochemical purity. The tracer was stable after incubating with rat serum at 37° C. for 24 hr. Biodistribution studies showed fast renal and liver/gastrointestinal clearance of the tracer. En face, Oil Red O and autoradiography studies indicated that 4 month ApoE^{-/-} mice fed a high fat diet had significantly higher tracer uptake than chow-fed ApoE^{-/-} or wild-type mice, while the uptake in the last two groups is similar. The percentage of lesion area measured from autoradiography, en face and Oil Red O showed good correlation. This Example shows that 7-¹²³I-O-Nec-1 may be used as a SPECT imaging agent, and has promising necroptosis targeting properties. The evaluation in ApoE^{-/-} mice shows that the tracer is taken up by more advanced atherosclerotic lesions.

### Introduction:

Cell death plays a pivotal role in the pathophysiology of many acute and chronic inflammatory diseases, including atherosclerosis. Atherosclerosis is primarily driven by the infiltration of inflammatory cells into the vessel wall where cholesterol has accumulated. Macrophages attempt to clear the lipid by phagocytosis, becoming inflammatory foam cells, and their emigration out of the plaque is impaired. Eventually, the accumulated macrophages undergo cell death, contributing to necrotic core regions within the atherosclerotic plaque, which ultimately contributes to its rupture and a cardiovascular event such as stroke or heart attack. The precise mechanism by which these cells undergo cell death within the plaque is an area of active investigation.

Thorough understanding of the complex mechanism of cell death is essential to design effective therapeutic strategies. Historically, cell death has been categorized as two distinct biochemical pathways, apoptosis and necrosis. Apoptosis is the programmed cellular death that may occur in multicellular organisms, and is characterized by multiple highly regulated steps including caspase activation and mitochondrial damage that leads to downstream events such as concomitant nucleus and cytoplasm condensation, DNA degradation, membrane blebbing, and caspase-mediated cleavage of various cellular proteins, ultimately resulting in a clean and controlled cell death. In contrast, necrosis is characterized by cell membrane and organelle disruption, cell swelling, mitochondrion impairment followed by cell lysis, ultimately resulting in an uncontrolled cell death that is usually accompanied by a host inflammatory response.

Although studies have demonstrated apoptosis activation in various diseases, necrosis remains the major component of pathological tissue injury in atherosclerosis, stroke, myocardial infarction, trauma, and neurodegeneration. An alternative death pathway called necroptosis is defined as “programmed necrosis”, and represents a regulated caspase-independent cell death pathway involving receptor-interacting protein kinase 1 (RIP1), receptor-interacting protein kinase 3 (RIP3) and mixed lineage kinase domain-like protein (MLKL). The recent discovery of a class of inhibitors (necrostatins) that can inhibit the activity of RIP3 kinase, the key driver of necroptosis, has fuelled the clear definition of the signaling pathways regulating necroptosis and has provided an exciting platform for developing therapeutic targets to diagnose and treat inflammatory and degenerative conditions (see, for example: Smith et al., 2011; Christofferson et al, 2010).

Necrostatins, including necrostatin-1 (Nec-1), Nec-3, Nec-4, Nec-5 and Nec-7, are low molecular weight molecules that have been synthesized as necroptosis inhibitors. The identification and optimization of necrostatins can assist in defining the role of necroptosis in disease pathophysiology, and serve as compounds for therapeutic development. In this Example, it has been found that these necrostatin molecules can be tagged with gamma or positron emitters, and these radiolabeled necrostatins can serve as non-invasive imaging tools to visualise sites of necroptosis in disease and monitor the necroptotic pathway, which will lead to the development of novel diagnostic agents based on state-of-art biomedical imaging techniques Single Photon Emission Computed Tomography (SPECT) and Positron Emission Tomography (PET).

¹¹C labeled Nec-3 compounds are the only radiolabeled necrostatins that have been developed to date as potential PET agents targeting necroptosis (Gao et al, 2010). Moreover, only radiolabeling chemistry has been reported, no in vitro biology, in vivo, and ex vivo pre-clinical animal studies have been described. Due to its short 20 minutes half-life, ¹¹C is inconvenient for radiolabeling and impractical for imaging application. Radiotracers labeled with long-lived isotopes can be developed for SPECT imaging. Wth the half-life (13.2 hrs) and optimal gamma energy (159 keV), the choice of ¹²³I was made for developing ¹²³I labeled SPECT tracers targeting necroptosis, with Nec-1 as the exemplified necrostatin molecule.

Teng, X. et al (2005) synthesized of Nec-1 derivatives and evaluated necroptosis inhibitory activity in Fas-associated death domain protein (FADD)-deficient variant of human Jurkat T cells treated with tumor necrosis factor-α (TNF-α). The structure-activity relationship (SAR) study revealed that several positions of the indole portion of Nec-1 were intolerant of substitution, while smaller substituents (i.e. OMe, Cl) at the 7-position resulted in increased activity (see 7-Cl-Nec-1, in Formula III, below).

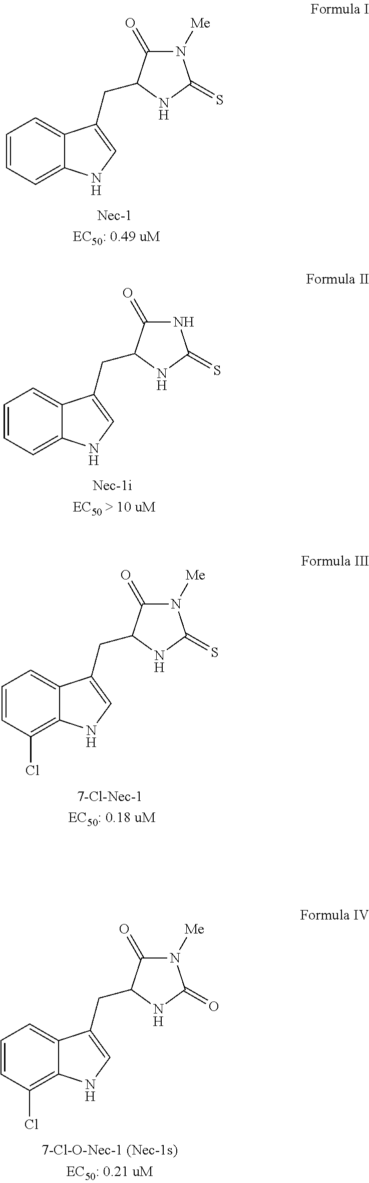 [SMILES: CN1C(=O)C(Cc2c[nH]c3ccccc23)NC1=S ; O=C1NC(=S)NC1Cc1c[nH]c2ccccc12 ; CN1C(=O)C(Cc2c[nH]c3c(Cl)cccc23)NC1=S ; CN1C(=O)NC(Cc2c[nH]c3c(Cl)cccc23)C1=O]

The hydantoin ring of Nec-1 was quite sensitive to structural modifications. For example, elimination of the methyl group in the hydantoin moiety completely abolished its anti-necroptotic activity, resulting in a so-called inactive form of the compound (Nec-1i, Formula II, above). See Teng et al., 2005 and Degterev et al., 2005. Changing the sulfur in the hydantoin ring to oxygen did not affect the necroptotic inhibitory activity, as indicated by the similar EC₅₀ values of the two derivatives 7-Cl-Nec-1 (Formula III) and 7-Cl-O-Nec-1 (Formula IV). 7-Cl-O-Nec-1, often referred as Nec-1s, is a more stable variant and ideal for in vivo animal studies (Takahashi et al., 2012).

In this Example, 7-¹²³I-O-Nec-1 is synthesized as the radioiodine version of 7-Cl-O-Nec-1 (Nec-1s). The synthesis of the precursor 7-Br-O-Nec-1 is reported in this Example, and its iodination with cold ¹²⁷I and hot ¹²³I, biodistribution studies, and preliminary evaluation in ApoE^{-/-} atherosclerosis mouse model.

### Materials and Methods

Materials. Unless otherwise noted, all chemicals and solvents were purchased from commercial sources and used without further purification. ¹²³I was provided by Nordion Inc. Analytical HPLC was performed on a Water's system with 1525 Pump, 2998 Photodiode Array Detector, and 717+Autosampler. Perkin Elmer's 150TR Flow Scintillation Analyzer was used as radiomatic detector. Capintec dose calibrator (CRC-25R) was used to measure the radioactivity. Dionex GP50 gradient pump, Water's Fraction Collector III, and Rheodyne six-port sample injection valve (7725i) were used for the HPLC purification of ¹²³I labeled product. For synthesis of small organic molecules, precursor and cold iodinated product, the purification was carried out by flash column chromatography using Silicycle silica gel (40-63 μm). Analytical thin layer chromatography (TLC) was performed on glass silica gel plate. Visualization was accomplished with UV light.

¹H NMR and ¹³C NMR spectra were recorded on Bruker AVANCE 300 MHz, 400 MHz or 500 MHz spectrometers at ambient temperature. Spectral data was reported in ppm using solvent as the reference (CDCl₃ at 7.26 ppm, or DMSO-d6 at 2.50 ppm for ¹H NMR and CDCl3 at 77.0 ppm or DMSO-d6 at 39.43 for ¹³C NMR). ¹H NMR data was reported as: multiplicity (s=singlet, d=doublet, t=triplet, m=multiplet), integration and coupling constant(s) in Hz. High-resolution mass spectroscopy (HRMS) was performed on a Kratos Concept-11A mass spectrometer with an electron beam of 70 ev at Queen's University, Kingston, Canada.

Preparation of 3-methylimidazolidine-2,4-dione (1). To a solution of hydantoin (0.50 g, 5.0 mmol) in toluene (30 mL), 1,1-dimethoxy-N,N-dimethylethanamine (1.3 g, 10 mmol, 2 equiv) was added. The mixture was refluxed for 2 hours then allowed to cool down to room temperature. Direct crystallization from the toluene gives 3-methylimidazolidine-2,4-dione (1) as a white solid (0.39 g, 68% yield) without further purification. ¹H NMR (400 MHz, CD₃OD): δ8.01 (s, 1H), 3.88 (s, 1H), 2.81 (s, 1H). ¹³C NMR (400 MHz, CD₃OD): δ172.9, 158.9, 46.0, 23.2.

Preparation of 5-((7-bromo-1H-indol-3-yl)methylene)-3-methylimidazolidine-2,4-dione (2). To a solution of 7-bromo-1 H-indole-3-carbaldehyde (220 mg, 1.0 mmol) in piperidine (2 mL) in a seal tube was added 3-methylimidazolidine-2,4-dione (1) (230 mg, 2.0 mmol, 2 equiv). The tube was sealed and the reaction mixture was heated for 6 hours at 110° C. After cooling down to room temperature, the mixture was transferred to water (100 mL) using a pipet. The target compound was filtered out as yellow precipitate (234 mg, 73% yield) without further purification. ¹H NMR (300 MHz, DMSO-d6): δ 8.19 (s, 1H), 7.80 (dd, J=8.02, 0.80 Hz, 1H), 7.38 (dd, J=7.59, 0.74 Hz, 1H), 7.04 (t, J=7.78 Hz, 1H), 6.77 (d, J=0.53 Hz, 1H), 2.93 (s, 3H).

Preparation of 5-((7-bromo-1H-indol-3-yl)methyl)-3-methylimidazolidine-2,4-dione (7-Br-O-Nec-1, 3). To a solution of 5-((7-bromo-1H-indol-3-yl)methylene)-3-methylimidazolidine-2,4-dione (2) (200 mg, 0.63 mmol) in a mixture of THF and H₂O (100 mL, 1:1 ratio) was added CoCl₂ (160 mg, 1.3 mmol, 2 equiv). After cooling down to 0 ° C. in ice water bath, the mixture was added NaBH₄ (360 mg, 9.4 mmol, 15 equiv) gradually. It was allowed to warm to room temperature and stirred for overnight. The THF was evaporated under reduced pressure and water was added to dissolve the residue. Two portions of methylene chloride (2×10 mL) were used to extract the product. The organic layer was combined and dried using anhydrous NaSO₄, concentrated under reduced pressure and purified by silica gel chromatography to give the titled product as a colorless solid (124 mg, 61% yield). ¹H NMR (300 MHz, CDCl₃): δ8.34 (s, 1H), 7.51 (d, J=7.94 Hz 1H), 7.35 (dd, J=7.61, 0.54 Hz, 1H), 7.11 (d, J=2.39 Hz, 1H), 6.99 (t, J=7.79 Hz, 1H), 5.73 (s, 1H), 4.25 (ddd, J=8.79, 3.77, 1.20 Hz, 1H), 3.40 (ddd, J=14.68, 3.74, 0.64 Hz, 1H), 2.98 (dd, J =14.80, 8.83 Hz, 1H), 2.93 (s, 3H). ¹³C NMR (500 MHz, CDCl3): δ173.4, 157.3, 134.9, 128.0, 124.8, 123.6, 121.1, 119.9, 118.4, 117.8, 57.9, 28.1, 24.5. HRMS Electron Impact (EI): exact mass calcd. for C₁₃H₁₂BrN₃O₂: 321.0107, found: 321.0122.

Preparation of 5-((7-lodo-1H-indol-3-yl)methyl)-3-methylimidazolidine-2,4-dione (7-¹²⁷I-O-Nec-1, 4). To a solution of 5-((7-bromo-1H-indol-3-yl)methyl)-3-methylimidazolidine-2,4-dione (7-Br-O-Nec-1, 3) (20 mg, 0.062 mmol) in dioxane (4 ml) in a seal tube was added CuSO₄ (18 mg, 0.11 mmol, 1.8 equiv.), Na₂S₂O₅ (21 mg, 0.11 mmol, 1.8 equiv.), Nal (28 mg, 0.19 mmol, 3 equiv.) and trans-N,N′-dimethylcyclohexane-1,2-diamine (17 mg, 0.12 mmol, 1.9 equiv.). The tube was sealed and the reaction mixture was heated at 130° C. for overnight. Afterwards it was cooled to ambient temperature, concentrated under reduced pressure and purified by silica gel chromatography to give corresponding compound as a colorless solid (18 mg, 79% yield). ¹H NMR (300 MHz, CDCl₃): δ8.21 (s, 1H), 7.56 (d, J=8.17 Hz, 2H), 7.15 (s, 1H), 6.90 (t, J=7.64 Hz, 1H), 5.35 (s, 1H), 4.27 (dd, J=9.42, 3.72 Hz, 1H), 3.42 (dd, J=14.14, 3.34 Hz, 1H), 3.00-2.89 (m, 4H). HRMS Electron Impact (EI): exact mass calcd. for C₁₃H₁₂IN₃O₂: 368.9969, found: 368.9982.

Synthesis of 5-((7-lodo (¹²³I)-1H-indol-3-yl)methyl)-3-methylimidazolidine-2,4-dione (7-¹²³I-O-Nec-1, 5). The following reagents were added into a 2 mL vial: 5-((7-bromo-1H-indol-3-yl)methyl)-3-methylimidazolidine-2,4-dione (7-Br-O-Nec-1, 3) (100 μL, 6 mg/mL in dioxane), trans-N,N′-dimethylcyclohexane-1,2-diamine (5 μL, 17.6 mg/mL in EtOH), CuSO₄ (5 μL, 20 mg/mL in H₂O), Na₂S₂O₅ (5 μL, 24 mg/mL in H₂O), Na¹²³I (20 μL in 0.1 M NaOH, 148-185 MBq (4-5 mCi)). The vial was sealed and the reaction mixture was heated at 130° C. for 30 minutes. After cooling at room temperature for 5 minutes, the reaction mixture was purified by reverse-phase HPLC at ambient temperature using a Luna C18(2), 5 μm, 100 Å, 250×4.6 mm column (Phenomenex, Calif., USA) and ethanol/water (40%/60%, v/v) as mobile phase, and a Dionex pump with flow rate 1 mL/min. The product at retention time 18-20 minutes was collected and heated at 60° C. under a constant supply of nitrogen to evaporate ethanol. A charcoal filter was used as a vent and also to absorb free ¹²³I. The purified product in water was analyzed by HPLC before injected to animals. The retention time (RT) of 7-¹²³I-O-Nec-1 (5) and free ¹²³I was ˜19.1 min and 2.2 min, respectively. The radiochemical purity was ≧95%. The overall yield after HPLC purification was ˜50%.

Serum stability testing of 7-¹²³I-O-Nec-1. Serum stability testing was carried out by mixing 25 μL of 7-¹²³I-O-Nec-1 (5) and 100 μL of rat serum at 37 ° C. for 24 hours. 200 pL cold EtOH was added afterwards, and the mixture was centrifuged at 2500 ×g at 4° C. for 15 minutes to precipitate proteins. The supernatant was injected into HPLC. The product was stable in serum, no de-iodination or degradation was found.

Biodistribution of 7-¹²³I-O-Nec-1 in mice and rats. All housing, handling and experimental procedures were in strict accordance with the guidelines of Canadian Council on Animal Care (CCAC) and with approval from Animal Care Committee (ACC) at the University of Ottawa, in Ottawa, Canada.

Biodistribution studies were carried out on C57BL/6 mice weighing 19-21 g and Sprague Dawley rats weighing 400-560 g (Charles River Laboratories, Mass., USA). On the day of experimentation, the animals were anesthetized and maintained under isofluorane during the injection. A tail vein catheter was inserted, 5.3-6.7 MBq of 7-¹²³I-O-Nec-1 was administered to the mice and 3.1-3.3 MBq of 7-¹²³I-O-Nec-1 was injected into the rats. The animals were sacrificed at 1 hr or 2 hr post injection (p.i.) (n=3 per group). The heart, liver, kidney, muscle, femur, spleen, blood, brain, intestine, lung, stomach, urine/bladder, ovaries/testes and thyroid were extracted, weighed and analyzed for total gamma counts (Wizard 2 2480 Automatic Gamma Counter, Perkin Elmer). The tissue uptake was decay corrected, and the percentage of injected dose per gram (% ID/g) was calculated.

Ex vivo autoradiography study with ApoE^{-/-} mice. Eight week-old female apolipoprotein E knockout (Apo E^{-/-}) mice were purchased from Charles River Laboratories (Charles River, Canada) and fed either a chow or western diet (TD.10885, Harlan Laboratories) for 2 months (n=3 per group). Four-month old wild type C57BL/6 mice (n=3), which do not develop atherosclerosis, were used as controls. 7-¹²³I-O-Nec-1 (37.0-55.5 MBq) was administrated intravenously into the mice under anesthetic. After 2 hr, the mice were euthanized in a CO₂ chamber. Using a Gilson Minipuls 2 peristaltic pump, the mice were perfused with 20 mL phosphate buffered saline (PBS) followed by 10 mL of 10% formalin via left ventricle cannulation. Perfusate was drained from a cut within the right atrium. Aorta was dissected from heart at the base using a dissecting microscope following removal of surrounding fat and connective tissue. For en face experiments aortic tissues were flattened by a longitudinal cut through one side of the aortic wall cutting through the carotids and the innominant artery until the aorta was split forming a Y-shaped structure.

The uptake and distribution of the tracer on aortic tissue was studied using digital autoradiography method. En face specimens were immediately exposed to super resolution phosphor screens in a Fisher Biotech autoradiography cassette. After overnight exposure at room temperature, the screens were scanned with a Cyclone Phosphor Imager (Perkin Elmer, Downers Grove, Ill.). Images were analyzed using OptiQuant™ 5.0 software. The regions of interest (ROI) were drawn around the lesions on the aortic arch, and the counts in Digital Light Unit (DLU) and surface areas in mm² were measured. The DLU was converted to activity in pCi using a set of calibration standards with known activities, which were exposed and scanned on the same screen used for the aorta samples. The percentage injected dose (% ID) was calculated from dividing the activity (pCi) of the lesion by the injected activity. Activity density in % ID/m² was calculated and normalized by animal body weight to get % ID×kg/m². The areas of the lesions and the whole aortic arch in mm² were also recorded using the OptiQuant™ 5.0 software, and the percentage of lesion within the area of the aorta vessel wall was calculated. For the normal C57BL/6 control mice, since no lesions were identified, the ROI were drawn around the whole aortic arch, and the tracer uptake in % ID×kg/m² was calculated as the average activity density within the arch.

En face and Oil Red O images. The en face aortic tissue was photographed prior and post-Oil Red O staining (for lipid content). Images were captured by Infinity 2 digital camera mounted to a dissecting microscope and analyzed using ImagePro™ software. Both unstained and Oil-Red-O stained images were utilized to quantify aortic lesion areas. Similar to the quantification method in autoradiography, the total surface area of the aorta was measured and the percentage of lesion within the area of aorta arch was calculated.

Statistical methods. The biodistribution and tracer uptake data are presented as mean±SD. GraphPad™ PRISM (San Diego, Calif.) was used for statistical analysis. To compare the tracer uptake between different groups of mice, two-tailed unpaired Student's t tests were performed. Differences at the 95% confidence level (p<0.05) were considered significant. Linear regression analysis and Pearson's correlation were performed for the percentage of lesion area measured from autoradiography, en face and Oil Red O images.

### Results

Synthesis of 7-¹²⁷I-O-Nec-1 and 7-¹²³I-O-Nec-1.

7-Br-O-Nec-1 (3) was designed as the precursor for iodination, and synthesized in a sequence described by Teng, X. et al, (2005), with some modifications, as shown in Scheme 1.

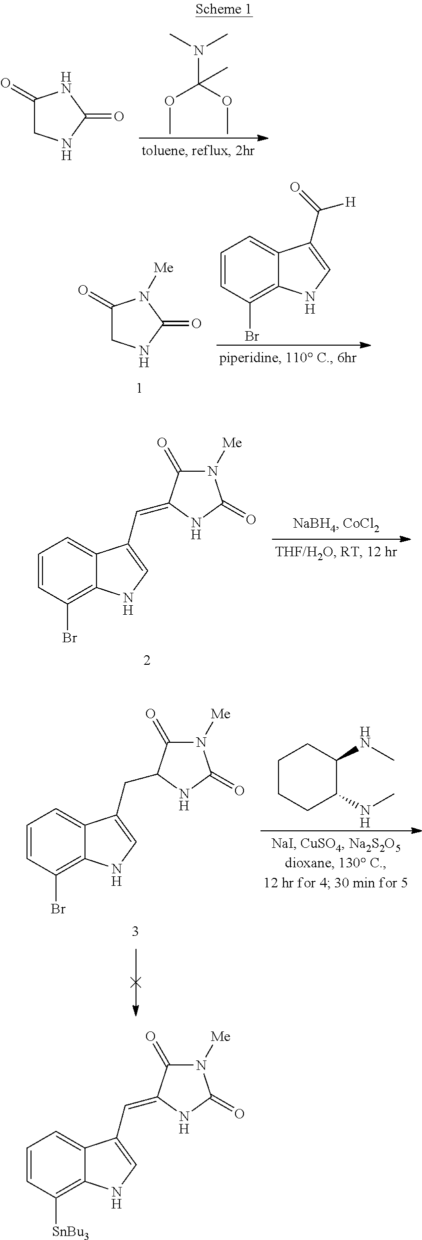

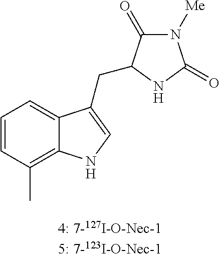 [SMILES: Cc1cccc2c(CC3NC(=O)N(C)C3=O)c[nH]c12]

The 3-methylhydantoin 1 was afforded by a S_{N}2 reaction of hydantoin in the presence of 1,1-dimethoxy-N,N-dimethylethanamine with 68% yield (see Janin et al., 2002). Compound 1 was then condensed with7-bromo-1H-indole-3-carbaldehyde in piperidine to give 5-((7-bromo-1H-indole-3-yl)methylene)-3-methylimidazolidine-2,4-dione (2) with 73% yield, which was reduced by NaBH₄ under CoCl₂ catalyzed condition to provide 7-Br-O-Nec-1 (3). Compound 3 was purified by silica gel column chromatography, and the yield was 61% after purification.

Cold 7-¹²⁷I-O-Nec-1 (4 in Scheme 1) was prepared by halogen exchange reaction between 7-Br-O-Nec-1 (3) and 3 equivalent of sodium iodide using in situ generated copper (1) iodide as catalyst, and 2 equivalent of trans-N,N′-dimethylcyclohexane-1,2-diamine as ligand (see Klapars and Buchwald, 2002) The reaction required high temperature (130° C.) heating for ˜12 hours to achieve ˜79% yield after purification by silica gel chromatography.

The ¹²³I-labeled Nec-1 compound (7¹²³I-O-Nec-1, 5) was synthesized by a similar halogen exchange procedure. Lower amount of diamine ligand, CuSO₄ and Na₂S₂O₅, and 30 minutes of heating were needed due to the trace amount of Na¹²³I involved and the smaller scale of the reaction. Analytical HPLC showed that the radiolabeling yield was ˜75%. The mixture was purified by HPLC using reverse phase C18 column and ethanol/water (40%/60%, v/v) as mobile phase.

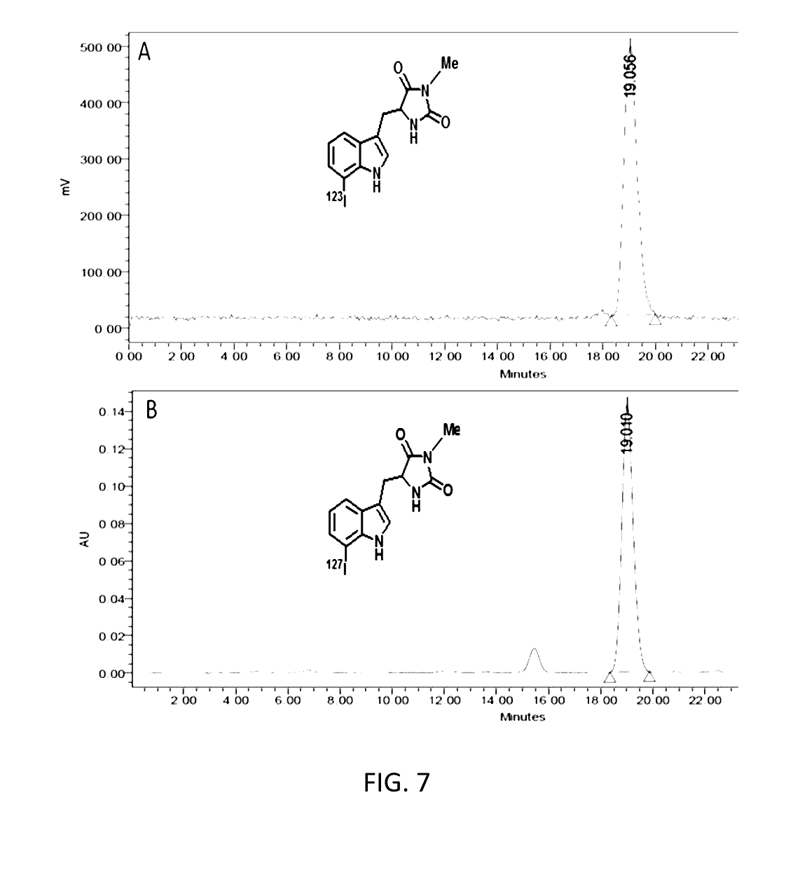

FIG. 7 illustrates HPLC chromatograms of 7-¹²³I-O-Nec-1 (radiomatic trace) (Panel A); and 7-¹²⁷I-O-Nec-1 (UV trace at 280 nm) (Panel B). The retention time of the starting material 7-Br-O-Nec-1 (3) and the labeled product 7-¹²³I-O-Nec-1 (5) was 15.5 min and 19.1 min (FIG. 7), respectively, providing sufficient room for complete separation. The radiochemical purity was >95%, as shown by HPLC. The overall radiolabeling yield after HPLC purification was -50%.

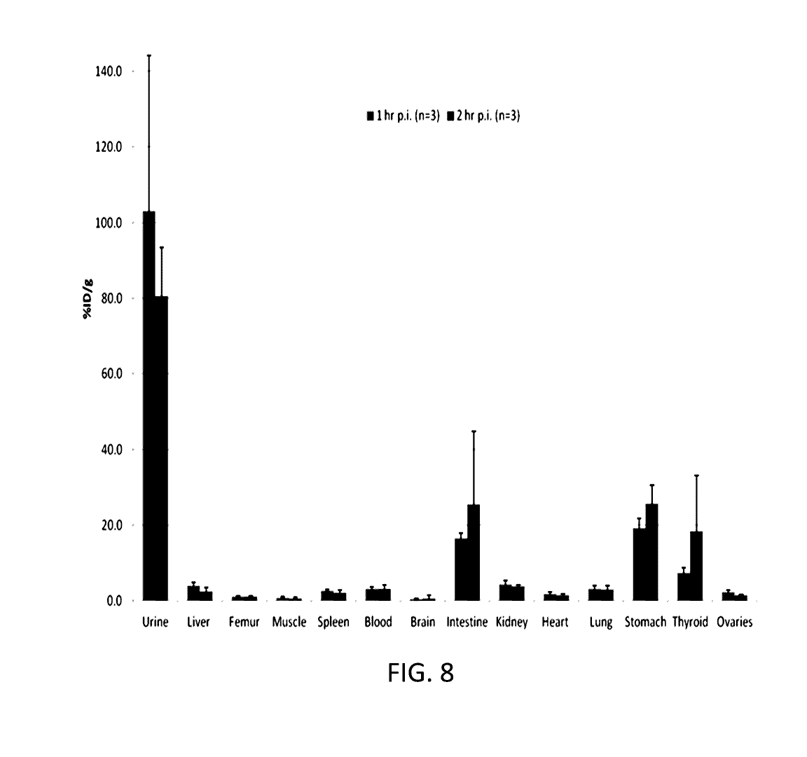

Biodistribution of the 7-¹²³I-O-Nec-1 tracer. Biodistribution of 7-¹²³I-O-Nec-1 in C57BL/6 mice at 1 hr and 2 hr post injection (p.i.) is shown in FIG. 8. The mice (19-21 g) were injected intravenously with 5.3-6.7 MBq of 7-¹²³I-O-Nec-1 in water. The error bars represent standard deviation (SD). The biodistribution of 7-¹²³I-O-Nec-1 (5) in normal C57BL/6 mice (Table 1 and FIG. 8) and Sprague-Dawley rats (Table 1) showed similar patterns. The tracer was excreted rapidly from the renal system, as indicated by the substantial amount of activity in urine. Significant uptake in intestine suggests that the tracer also cleared from the liver/gastrointestinal system. The thyroid and stomach uptake was high and increased from 1 hr to 2 hr p.i., indicating de-iodination over time.

TABLE 1
Biodistribution of 7-¹²³I-O-Nec-1 in female C57BL/6 mice at 1 hr and 2 hr
post injection (p.i.) and in male Sprague-Dawley rats at 2 hr p.i. (n = 3 per
group)

| Tissues | Mice (1 hr p.i.) | Mice (2 hr p.i.) | Rats (2 hr p.i.) |
|---|---|---|---|
| Urine | 103.153 ± 41.005 | 80.627 ± 12.850 | 2.513 ± 0.282 |
| Liver | 4.030 ± 0.824 | 2.562 ± 0.916 | 0.226 ± 0.028 |
| Femur | 1.167 ± 0.214 | 1.189 ± 0.205 | 0.052 ± 0.009 |
| Muscle | 0.859 ± 0.278 | 0.723 ± 0.307 | 0.048 ± 0.005 |
| Spleen | 2.657 ± 0.390 | 2.173 ± 0.728 | 0.106 ± 0.009 |
| Blood | 3.149 ± 0.547 | 3.136 ± 1.024 | 0.153 ± 0.031 |
| Brain | 0.429 ± 0.170 | 0.640 ± 0.847 | 0.023 ± 0.004 |
| Intestine | 16.578 ± 1.224 | 25.573 ± 19.206 | 0.867 ± 0.062 |
| Kidney | 4.296 ± 1.084 | 3.841 ± 0.429 | 0.236 ± 0.016 |
| Heart | 1.868 ± 0.519 | 1.405 ± 0.484 | 0.098 ± 0.007 |
| Lung | 3.248 ± 0.788 | 2.975 ± 1.025 | 0.143 ± 0.013 |
| Stomach | 19.294 ± 2.556 | 25.711 ± 4.877 | 1.685 ± 0.395 |
| Thyroid | 7.344 ± 1.469 | 18.387 ± 14.764 | 1.407 ± 0.189 |
| Ovaries/testis | 2.349 ± 0.570 | 1.462 ± 0.184 | 0.089 ± 0.009 |

C57BL/6 mice (19-21 g) were injected intravenously with 5.3-6.7 MBq of 7-¹²³I-O-Nec-1 in water.
Sprague-Dawley rats (400-560 g) were injected intravenously with 3.1-3.3 MBq of 7-¹²³I-O-Nec-1 in water.
The organ uptake values are reported as percentage injected dose per gram (% ID/g) and presented as mean ± SD.

7-¹²³I-O-Nec-1 localizes to atherosclerotic lesions in ApoE^{-/-} mice. Apolipoprotein E knockout (ApoE^{-/-}) mice were used to evaluate the uptake of the tracer 7-¹²³I-O-Nec-1 in atherosclerosis lesions. Three groups of 4 month old mice (n=3 per group) were examined. The first group of mice were fed a western high fat diet (HFD) for 2 months (4 month ApoE^{-/-} mice with HFD), and were expected to have significant amount of lesions developed in the aortic arch. The second group of mice were ApoE^{-/-} mice fed a chow diet for 2 months (4 month ApoE^{-/-} mice), and develop less lesion areas and/or less severe lesions compared to the HFD-fed mice. The third group of mice were wild type C57BL/6 mice without atherosclerosis and used as normal controls (4 month control).

Tracer uptake results were evaluated for the three groups of mice. The uptake values in % ID×kg/m² were calculated as: 1) the average of 15 lesions for the 4 month ApoE^{-/-} mice with HFD; 2) the average of 7 lesions for the 4 month ApoE^{-/-} mice; 3) the average tracer uptake of whole aortic arch for 4 month control mice.

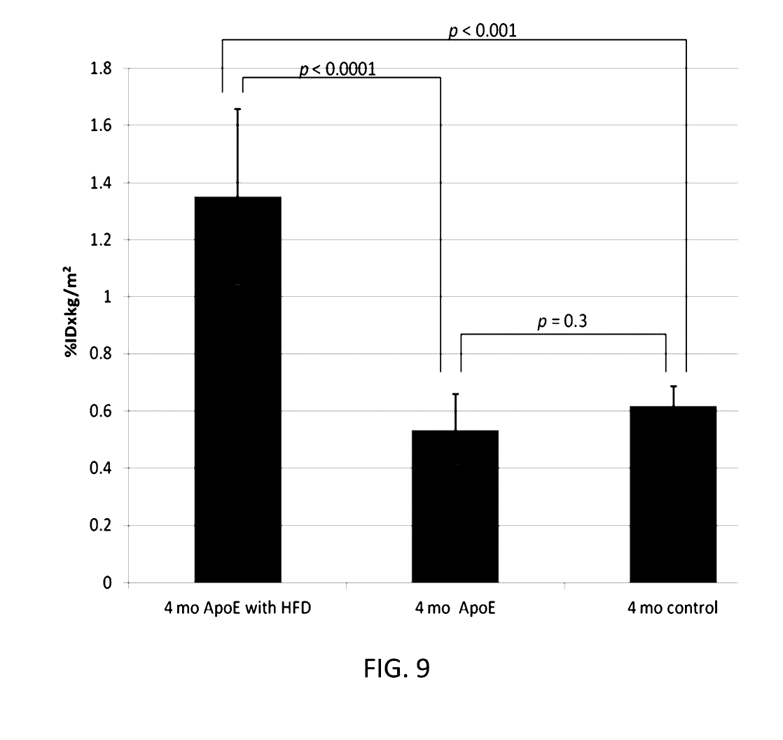

FIG. 9 shows comparison of aortic lesion uptake in 4 month old ApoE^{-/-} mice fed with a western high fat diet (HFD) for 2 months (4 mo ApoE with HFD), 4 month old ApoE^{-/-} mice fed with a chow diet for 2 months (4 mo ApoE), and 4 month old C57BL/6 control mice (4 mo control) (n=3 per group). As indicated in FIG. 9, the tracer had significantly higher uptake in the aortic lesions for the 4 month ApoE^{-/-} mice with HFD, compared to same age of mice under chow diet (p<0.0001) and the normal control mice (P<0.001). While the difference between the lesion uptake of the 4 month ApoE^{-/-} mice and the average aortic arch uptake of the control mice was not statistically significant (p=0.3).

FIG. 10 shows en face, Oil Red O and autoradiography images of a 4 month old ApoE^{-/-} mouse fed with a western high fat diet (HFD) for 2 months (Panel A); and a 4 month old C57BL/6 control mouse (Panel B). FIG. 10 demonstrates en face, Oil Red O and autoradiography images of a representative aorta of a 4 month ApoE^{-/-} mouse with HFD (Panel A) and a control mouse (Panel B). En face and Oil Red O images showed 4 month ApoE^{-/-} mouse with HFD had several lesions, while the 4 month control mouse had no lesions identified. Correspondingly, the tracer activity is more intense for the 4 month ApoE^{-/-} mouse with HFD in the autoradiography images, as compared to the 4 month control mouse. It is noted that some lesions on the en face and Oil Red O images did not have obvious lesions shown on the autoradiogram.

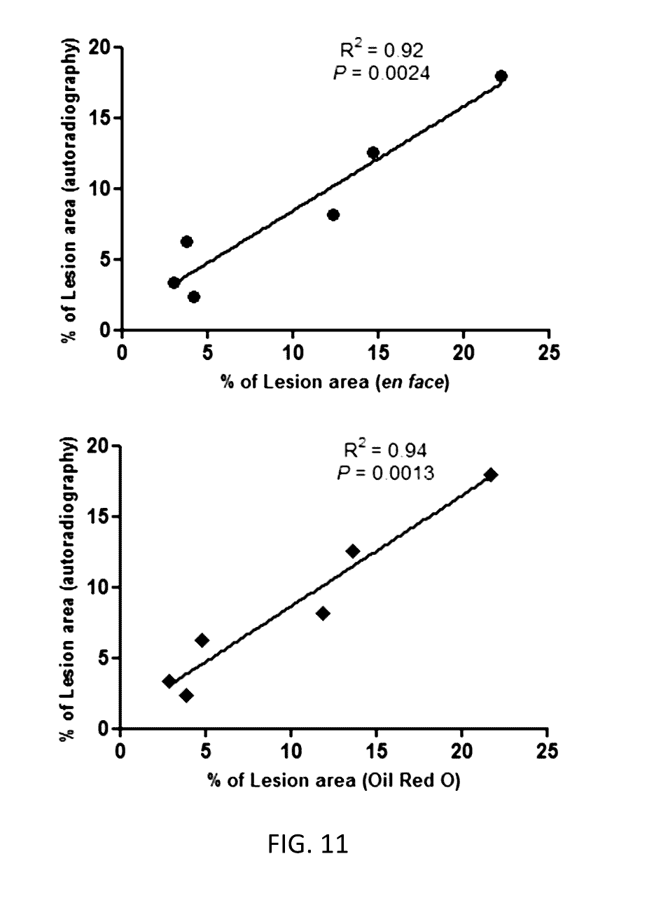

FIG. 11 shows correlation of lesion area measured from en face, Oil Red O (upper panel) and autoradiography (lower panel) images. The three spots at lower percentages are from the 4 month ApoE^{-/-} mice fed with a chow diet, and the three spots at higher percentage are from the 4 month ApoE^{-/-} mice fed with HFD. The percentage of lesion area (including all lesions) related to whole aortic arch area was calculated for en face, Oil Red O and autoradiography images. As shown in FIG. 11, the percentage of lesion area from en face and Oil Red O correlates well with autoradiography measurement with R² values 0.92 and 0.94, respectively.

### Discussion

As illustrated in this Example, necroptosis is a form of programmed cell death that can be specifically inhibited by necrostatins. Unlike apoptosis, of which the signaling pathways have been well established, the precise underlying mechanisms of necroptosis remain less well understood. Radiolabeled tracers targeting necroptosis can be used to track and understand signaling pathways, are useful as diagnostic agents, and can guide the development of therapeutic drugs.

In this Example, radiolabeled necrostatin derivatives are formed, which can be used tracers for imaging necroptosis. Radiolabelled Nec-1 is formed with ¹²³I and biological evaluation was conducted. It is noted that changing the sulfur on the hydantoin ring with oxygen and substitution at the 7-position resulted in similar or higher necroptosis inhibitory activity. In this Example, radiolabel O-Nec-1 (Nec-1 with sulfur replaced by oxygen on the hydantoin ring) at 7-position was used to make the target tracer 7-¹²³I-O-Nec-1.

7-Br-O-Nec-1 (3) was synthesized and characterized using a similar procedure for 7-Cl-O-Nec-1. Since aromatic trialkyltin compounds have been widely used as precursors for radioiodination through room temperature iodo-destannylation reaction in this Example, a substitution of the bromine at 7-position of 3 with tributyltin was utilized (see Scheme 1). The reaction between 3 and bis(tributyltin) ((SnBu₃)₂) using bis-benzonitrile palladium chloride PdCl₂(PhCN)₂ as catalyst and tricyclohexylphosphine (PCy₃) as ligand only gave <10% yield, and the product was not stable at room temperature. This may have been due to the bulky tributyltin moiety causing steric hindrance at the 7-position on the indole ring.

Due to the difficulty of synthesizing 7-tributyltin derivative of Nec-1, 7-Br-O-Nec-1 (3) was chosen as the precursor for iodination through halogen exchange reaction. A common preparative method of converting aryl bromide into aryl iodide is a copper-catalyzed halogen exchange reaction, which is usually limited by harsh reaction conditions, such as high temperature (>150 ° C.) and large excess of copper (I) iodide. Klapars and Buchwald (2002) reported a method utilizing a catalyst system comprising Cul and a 1,2- or 1,3-diamine ligand. The diamine ligand strongly accelerates the reaction, and significantly increases the reaction yield. The reaction temperature was reduced to 110° C. and 130° C. for compounds with more steric hindrance at the halogen exchange site. This method was adapted, using trans-N,N′-dimethylcyclohexane-1,2-diamine, the most active ligand reported by Klapars and Buchwald (2002). Due to the steric hindrance at the 7-position of indole ring, the reaction was conducted at 130° C. for 12 hours in order to prepare cold iodinated compound 7-¹²⁷I-O-Nec-1 (4) with high yield. For radiolabeling reaction, since tracer level of Na¹²³I is involved, only 30 minutes is enough to achieve -75% yield. Instead of copper (I) iodide, which is not stable at room temperature, an in situ generated Cu(I) catalyzed condition was used, by reacting CuSO₄ with the reductant Na₂S₂O₅.

The tracer 7-¹²³I-O-Nec-1 was purified by reverse phase HPLC to remove free ¹²³I, impurities, and the precursor 7-Br-O-Nec-1, resulting in high radiochemical purity (>95%) and high specific activity. As shown in FIG. 7, the identification of the ¹²³I labeled tracer is confirmed by matching retention time of 7-¹²³I-O-Nec-1 and cold 7-¹²⁷I-O-Nec-1. The small peak at −15.5 min in FIG. 7 (Panel B) is from the starting material 7-Br-O-Nec-1. NMR and Mass Spectrometry characterization of 7-¹²⁷I-O-Nec-1 (data not shown) confirmed the expected chemical structure.

In vitro stability testing in rat serum indicated 7-¹²³I-O-Nec-1 is stable at 37 ° C. for 24 hrs with no de-iodination and no degradation. Biodistribution study in normal mice and rats demonstrated some degree of in vivo de-iodination. Biodistribution also showed fast renal and liver/gastrointestinal clearance of the tracer.

Necroptosis has been implicated in mediating certain disease conditions (Zhou et al., 2014; Linkermann et al., 2014). It is illustrated in this example that Nec-1 is useful as a therapeutic target and for diagnostic purposes. The 7-¹²³I-O-Nec-1 tracer is a specific exemplary diagnostic molecule, as its uptake in atherosclerotic lesions can be evaluated in atherosclerosis or other cardiovascular or cardiometaboilc disorders.

The apolipoprotein E deficient (ApoE^{-/-}) mouse is well established model of atherosclerosis due to its reliability, convenience and many practical aspects. ApoE^{-/-} mice fed with standard chow diet can develop atherosclerotic lesion in the ascending aorta at 4-5 months of age. When high fat Western diet (HFD) is given to ApoE^{-/-} mice, more advanced and complex lesions are developed at the same age. In this Example, three groups of 4 month old mice are examined: ApoE^{-/-} fed a HFD, ApoE^{-/-} fed a chow diet, and wild type C57BL/6 mice fed a chow diet (controls). Ex vivo autoradiography was conducted to quantify the tracer uptake, which was expressed as activity density normalized by body weight (% ID×kg/m²), using a previously published method (Zhao et al., 2007). It is established in this example that the aortic lesion uptake of 7-¹²³I-O-Nec-1 in ApoE^{-/-} mice with standard chow diet is significantly lower than the uptake in ApoE^{-/-} mice with HFD, but similar as the uptake in control mice (FIG. 9). This indicated that: a) there is some background tracer uptake in the vessel wall of normal mice; b) the aortic uptake of the tracer in the 4 month ApoE^{-/-} mice fed a chow diet, which have mild lesions only, is the same as background. Thus, the tracer is useful to detect more advanced and complex atherosclerotic lesions observed in ApoE^{-/-} mice fed a HFD.

Comparison of the en face and Oil Red O and autoradiography images (FIG. 10) demonstrate that the HFD fed ApoE^{-/-} mice had numerous lesions and higher tracer uptake compared to control. Upon closer examination of the distribution of aortic lesions, it was found that the some lesions shown on the en face and Oil Red O images do not have corresponding lesion uptake on the autoradiography image, and the lesion areas were not identical. This may be related to the various stages and complexities of the lesions, and the necrostatin tracer was only taken by more advanced lesions. In terms of the percentage of lesion area related to the whole aortic arch for the two groups of ApoE^{-/-} mice, the en face and Oil Red 0 measurements both correlate well with the autoradiography measurement (FIG. 11). As expected, the ApoE^{-/-} mice with standard chow diet had lower percentage of lesion area compared to the ApoE^{-/-} mice with HFD. The results in this Example show that necroptosis is likely to occur or contribute to the formation of advanced atherosclerotic lesions. Given that the ¹⁸F-FDG is one of the few clinically-used radiotracers to detect atherosclerosis, the data in this Example show 7-¹²³I-O-Nec-1 to be a useful tracer for identifying atherosclerotic plaques by SPECT imaging.

### Conclusion

An ¹²³I labeled necrostatin-1 tracer 7-¹²³I-O-Nec-1 was produced and evaluated in this Example, and was found to be stable in vitro in serum and shows limited in vivo de-iodination. Derivatization at 7-postion of Nec-1 with small substituents resulted in increased necroptosis inhibitory activity, the biological function of Nec-1 substituted with the large iodine atom makes this compound useful as a radiotracer. Further, 7-Fluorination can be undertaken, and 7-¹⁸F-O-Nec-1 can be used as a PET tracer. The evaluation in ApoE^{-/-} mice in this Example indicates the necroptosis targeting properties of the tracer, revealing use in diagnostic and therapeutic applications.

### Example 3

### Targeting Macrophage Necroptosis for Therapeutic and Diagnostic Interventions in Atherosclerosis

### Overview

Atherosclerosis results from a maladaptive inflammation driven primarily by macrophages, whose recruitment and proliferation drive plaque formation and progression. In advanced plaques, macrophage death contributes centrally to the formation of plaque necrosis, which underlies the instability that promotes plaque rupture and myocardial infarction. As such, targeting macrophage cell death pathways may offer promise for the stabilization of vulnerable plaques. Necroptosis is a recently discovered pathway of programmed cell necrosis regulated by RIP3 and MLKL kinases that in contrast to apoptosis, induces a pro-inflammatory state. We show herein that necroptotic cell death is activated in human advanced atherosclerotic plaques and can be targeted in experimental atherosclerosis for both therapeutic and diagnostic interventions. In humans with unstable carotid atherosclerosis, expression of RIP3 and MLKL is increased and MLKL phosphorylation, a key step in the commitment to necroptosis, is detected in advanced atheromas. Investigation of the molecular mechanisms underlying plaque necroptosis showed that atherogenic forms of LDL increase RIP3 and MLKL transcription and phosphorylation—two critical steps in the execution of necroptosis. Using a radiotracer developed with the necroptosis inhibitor Nec-1, we show that ¹²³I-Nec-1 localizes specifically to atherosclerotic plaques in Apoe−/− mice, and its uptake is tightly correlated to lesion areas by ex vivo nuclear imaging. Furthermore, treatment of Apoe−/− mice with established atherosclerosis with Nec-1 reduced lesion size and markers of plaque instability, including necrotic core formation. Macrophage cell death and necrotic core formation in atherosclerosis can be used as both a diagnostic and therapeutic tool for the treatment of unstable plaques in atherosclerosis.

### Introduction

Atherosclerosis is characterized by the accumulation of lipid-rich plaques in medium to large arteries, which are replete with macrophages, T-lymphocytes, lipids and cholesterol crystals. Atherosclerosis is considered a benign disease until plaques weaken and rupture, leading to acute thrombus and subsequent myocardial infarction or stroke. A hallmark of such vulnerable lesions is the presence of a large necrotic core covered by a thin fibrous cap, which renders the plaque susceptible to rupture. While the processes that underlie the initiation of inflammatory fatty lesions within the arterial wall are well understood, the mechanisms by which these benign lesions develop into rupture-prone culprit lesions are not. There is thus an urgent need to better understand the pathways that contribute to necrotic core formation, and to develop strategies to target these processes therapeutically.

Atherosclerosis is initiated by the accumulation of excess low-density lipoprotein cholesterol that becomes trapped in the subendothelial space, where it is modified in the oxidant rich environment. According to the oxidation hypothesis, oxidized LDL (oxLDL) activates innate immune cells, particularly macrophages, to engulf the modified-self LDL via constitutively expressed scavenger receptors on their cell surface, triggering the activation of pattern recognition receptors such as the Toll-like receptors and the inflammasome. OxLDL can be cytotoxic and induce apoptosis of macrophages and smooth muscle cells, which in early plaques, are effectively cleared by macrophages via efferocytosis. However, as macrophages accumulate lipid and undergo ER stress, efferocytosis has been shown to become defective, resulting in secondary necrosis and the uncontrolled release of inflammatory mediators, proteases and coagulation factors—all factors that promote plaque vulnerability. Thus, macrophage cell death has been considered to be a major contributor to atherosclerosis and necrotic core formation in plaques.

The discovery and characterization of a pathway of programmed necrosis, or ‘necroptosis’ has expanded the understanding of the mechanisms leading to cell death. It is now understood that apoptosis and necroptosis have evolved as counterbalances in the first line of defense against inflammatory stimuli, either exogenous or self-derived. When pro-apoptotic caspase-8 is inhibited or overwhelmed, either through synthetic or naturally occurring inhibitors, the kinases RIP1 and RIP3 become phosphorylated, leading to recruitment and activation of MLKL by RIP3, and loss of plasma membrane integrity resulting in the release damage-associated molecular patterns (DAMPs) into the extracellular space. The upstream stimuli of necroptosis are beginning to be defined, and it is clear that both self and non-self ligands can contribute. Necroptosis and apoptosis share many overlapping factors; however, the small molecule necrostatin-1 (Nec-1) uniquely inhibits the interaction of RIP1-RIP3 and subsequent downstream effectors of necroptosis, thus, Nec-1 can be considered a preferential inhibitor of necroptosis rather than apoptosis. The necrostatin class of small molecules has enabled the evaluation of RIP1-RIP3 in many pathologies, and next-generation necrostatins have increased specificity for RIP1.

In this Example, it is tested whether necroptosis is activated in advanced atherosclerosis in humans and whether it can be targeted for diagnostic and therapeutic intervention in a model of established atherosclerosis. The results of this Example illustrate that the necroptotic pathway is triggered in human atherosclerotic plaques and is associated with markers of lesion vulnerability. Atherogenic ligands drive the expression of necroptotic genes, and this can be targeted as a therapeutic and diagnostic tool for advanced atherosclerosis in vivo. These data show that necroptosis underlies plaque vulnerability in humans and that dually targeting necroptosis for therapeutic and diagnostic interventions may benefit patients at high risk for vulnerable plaques and downstream adverse clinical events.

### Materials and Methods

Reagents. HI-TBAR oxidized human LDL (BT-910X) and human LDL (BT-903) was purchased from Biomedical Technologies Inc (USA). M-CSF and mouse IL-1β Duoset ELISA kit were purchased from R&D Systems (USA). zYVAD.fmk and zVAD.fmk were purchased from BioVision, Inc (USA) and ApexBio (USA) respectively. Necrostatin-1 and Diphenyleneiodonium (DPI) were obtained from Sigma Aldrich. Cellular Reactive Oxygen Species (ROS) Detection kit was purchased from Abcam.

Human atherosclerotic lesion analysis. For gene expression studies, human arterial samples were obtained from the Biobank of Karolinska Endarterectomy (BiKE) at the Centre for Molecular Medicine, Karolinska Institute. In brief, control normal arteries (undiseased macroscopically atherosclerosis free-arteries, iliac and one aorta) were obtained from organ donors without any current or history of cardiovascular disease. Atherosclerotic plaques were obtained from patients undergoing surgery for stable or unstable carotid stenosis. All samples were collected with informed consent and the study was approved by the Ethical Committee of Northern Stockholm. Plaque tissues were ruptured with a Tissue Rupturer/Homogenizer (Omni Inc) and RNA isolated. Gene expression profiles were obtained from n=10 control arteries and n=127 atherosclerotic plaque samples and statistical analysis was performed using Students' t-test with correction for multiple comparisons (p<0.05). For details on patient characteristics. For immunohistochemical analysis, human coronary artery samples were obtained from the CVPath Institute Sudden Cardiac Death registry (CVPI-SCDr). Sudden death is defined as symptoms commencing within 6 hours of death (witnessed arrest) or death occurring within 24-hours after the victim was last seen alive in his/her normal state of health. Comprehensive analysis of coronary artery histology was performed for each subject. Formalin-fixed paraffin embedded coronary artery blocks were further cross-sectioned at 5 μm thickness for analysis.

After careful examination of hematoxylin & eosin and Movat pentachrome stained sections, advanced fibroatheromas and pathological intimal thickening control lesions were selected. H&E images were obtained on Axio Scan.Z1 (Carl Zeiss). Immunohistochemistry for phosphorylated MLKL was performed using a primary antibody against human pMLKL (rabbit monoclonal, Abcam) with a secondary conjugated to biotin and visualized using DAB (Sigma). Quantification of pMLKL positive areas was performed using ImageJ and statistical significance between the two groups were analyzed using unpaired Student's t-test (p<0.05).

Mice. C57BL6 wildtype (WT) and Apoe−/− and Casp1−/− mice were purchased from Jackson Laboratories. Rip3−/− mice were obtained from Genentech (San Francisco, Calif.).

Bone-marrow derived macrophages. Bone-marrow derived macrophages (BMDMs) were isolated from femurs of adult VVT or Rip3−/− mice and differentiated into macrophages using DMEM supplemented with 10% FBS and 1% penicillin-streptomycin plus either 20% L929 conditioned media or 20 ng/mL mouse M-CSF for 7-10 days. To polarize macrophages to M1 or M2, BMDMs were incubated on day 7 with 1 μg/mL LPS and 100 ng/mL IFNγ or 10 ng/mL IL-4 respectively for 24 h. Damage Associated Molecular Patterns (DAMPs) were obtained by subjecting BMDMs to 3×30 min freeze-thaw cycle or treatment with 100 ng/mL LPS and 50 μM zVAD.fmk for 24 hours, after which the media was collected and added to naïve cells at indicated ratios.

Cell viability assays. Cell death was determined by measuring lactate dehydrogenase (LDH) release into the media. Briefly, cells were treated with 100 ug/ml oxLDL in the presence or absence of 25 uM zVAD.fmk or 50 uM necrostatin-1 (Nec-1) for 24 hours and media was collected and centrifuged to pellet cell debris. The amount of LDH in media was measured in a kinetic assay by adding PBS containing 0.02% NADH and 0.03% sodium pyruvate and measuring absorbance at 340 nm for 10 minutes at 1 minute intervals. The slope of the curve provides a measure of cell death, which was expressed as fold change relative to control.

RNA isolation and quantitative real-time PCR. BMDMs were treated with control media with or without oxLDL or oxLDL+zVAD.fmk for the indicated time points with or without 1 h pre-treatment with 50 uM DPI. Alternatively, BMDMs were polarized to M0, M1 or M2 prior to RNA isolation, as described above. Total RNA was isolated using TRIzol reagent (Invitrogen) as per manufacturer's instructions and cDNA was synthesized using iScript Reverse Transcription kit (Biorad). Quantitative real-time PCR was performed in triplicate using either the Sso Advanced Universal SYBR Green Supermix (Biorad) or Taqman® Gene Expression Assay and mRNA level of target genes was normalized to HPRT or β-actin house keeping genes.

Western blot analysis. For western blot analysis of RIP3, cells were lysed in 1.25× sample buffer (83 mM Tris pH 6.8, 6.7% SDS, 13.3% Glycerol, 1.3% β-mercaptoethanol, 0.03% bromophenol blue) and boiled at 100° C. for 5 minutes prior to being subjected to SDS-PAGE and western blot analysis. PVDF membranes were blocked with 5% skim milk, followed by incubation with RIP3 (ProSci Inc, 1:500) or HSP90 (Santa Cruz Biotechnology, 1:1000) antibodies. For analysis of MLKL protein, cells were lysed in ice-cold M2 lysis buffer (50 mM NaF, 20 mM Tris, pH 7.0, 0.5% NP40, 250 mM NaCl, 3 mM EDTA, 3 mM EGTA, supplemented with Roche protease and phosphatase inhibitor cocktails) for 30 mins before centrifugation to pellet insoluble component. Samples were mixed 3:1 with 4× sample loading buffer (Bio-rad) prior to SDS PAGE and western blot analysis. PVDF membranes were blocked in 5% BSA and probed with MLKL (Millipore, 1:500) or GAPDH (Millipore, 1:1000) antibodies. Goat anti-mouse (1:2500) or anti-rabbit (1:5000) or anti-rat (1:2500) IRDye® secondary antibodies (Rockland) were utilized. The protein bands were visualized using Odyssey Infrared Imaging System (LI-COR Biotechnology).

Electron microscopy. Cultured cells were fixed in 2.5% glutaraldehyde in 0.1M cacodylate buffer (pH 7.5) at 4° C. Fixed cells were washed in 0.1M cacodylate buffer, postfixed in 1% OsO₄ and rinsed with 0.1M cacodylate buffer and distilled water. Fixed cells were lifted with 50 mM EDTA/HBSS and collected by centrifugation. Cell sediments were washed in 1XPBS, dehydrated in an ethanol series and embedded in Spurr's resin. Resin blocks were sectioned by ultramicrotome Leica EM UC6 using a diamond knife. Ultra-thin sections were mounted on copper grids coated with formvar film. Sections were stained with 2% alcoholic uranyl acetate and Reynold's lead citrate. Stained sections were examined with a Transmission Electron Microscope (TEM) JEOL 1230.

Measurement of Reactive Oxygen Species (ROS). BMDMs were pre-incubated with DPI for 30 minutes followed by the cell permeant reagent 2′, 7′-dichloroflurescin diacetate (DCFDA) fluorescent dye for 1 h. Cells were then treated with oxLDL or oxLDL+zVAD.fmk and fluorescence was measured at 3 h and 24 h as per manufacturers' instructions (Abcam, ab113851).

Promoter Luciferase Assay. Luciferase reporter constructs containing the human MLKL and RIP3 promoters were purchased from SwitchGear Genomics (Carlsbad, CA). RAW264.7 (ATCC) macrophages were plated in 24-well plates in 10% FBS DMEM and transfected using Lipofectamine 2000 (Life Technologies) with MLKL-promoter or RIP3-promoter plasmids (renilla-based luciferase reporter) together with pGL3 basic (firefly-based reporter, Promega) to normalize for transfection efficiency. Twenty-four hours after transfection, cells were treated with or without 100 ug/ml oxLDL as above, for an additional 24 hours. Cells were harvested in 1× passive lysis buffer and luciferase measured using the Dual Luciferase Assay (Promega) according to manufacturer's instructions. All experiments were performed in quadruplicate technical replicates at least 3 times.

Atherosclerosis Studies. All animal experiments were performed in accordance with the Animal Care and Use Committee, University of Ottawa, Canada. Eight week old apoE−/− mice (total n=17 mice) were fed an adjusted calories diet (21% fat; 0.2% cholesterol, Harlan Teklad) for 4 weeks to induce atherosclerotic progression. Mice were then injected with time-release placebo or stable Necrostatin-1 (Nec-1)15 tablets (2 mg/kg/day), which were synthesized by Innovative Research of America, USA. Mice were euthanized after 6 weeks of treatment, perfused with saline and aortic roots were embedded in OCT medium and frozen. Aortic roots were sectioned (10 μm) and stained with hematoxylin and eosin for lesion area and necrotic core quantification, and a minimum of 10 sections per animal were measured across the length of the entire aortic root. For en face aortic lesion quantifications, aortas were dissected removing all branching vessels down to the femoral bifurcation and then sliced ventrally and images of aortas were digitally captured using ImageProTM. Lesion areas within the entire length of the aorta were quantified using Image JTM and expressed relative to the total aortic surface area. Immunohistochemistry to visualize macrophages and smooth muscle cells was performed using primary antibodies to CD68 (rat monoclonal, Serotec) or phosho-MLKL (rabbit polyclonal, Abcam) with a secondary conjugated to biotin and α-smooth muscle actin (monoclonal, Sigma) conjugated to alkaline phosphatase and visualized using DAB or SigmaFAST Red (Sigma), respectively. Image J was used to quantify positive staining area and statistical significance between the two groups were analyzed using Students' t-test (p<0.05)

Radiochemistry and ex vivo Autoradiography study with ApoE−/− mice. The tracer ¹²³I-Nec-1 (7-¹²³I-Nec-1) was synthesized by a Cu(I) catalyzed direct halogen exchange reaction with the precursor 7-Br-Nec-1 and Na¹²³I (see attached companion manuscript for full details). Eight week-old female apolipoprotein E knockout (Apoe−/−) mice were purchased from Charles River Laboratories and fed either a chow or western diet (TD.10885, Harlan Laboratories) for 2 months (total n=6 mice). 7-¹²³I-O-Nec-1 (37.0−55.5 MBq) was administrated intravenously into the mice under anesthetic. After 2 hr, the mice were euthanized and perfused with PBS followed by 10% formalin via left ventricle cannulation. Aorta was dissected from heart at the base using a dissecting microscope following removal of surrounding fat and connective tissue. En face specimens were immediately exposed to super resolution phosphor screens in an autoradiography cassette, as previously described 46. After overnight exposure at room temperature, the screens were scanned with a Cyclone Phosphor Imager (Perkin Elmer, Downers Grove, Ill.). Images were analyzed using OptiQuant 5.0 software. The regions of interest (ROI) were drawn around the lesions on the aortic arch, and the counts in Digital Light Unit (DLU) and surface areas in mm² were measured. The DLU was converted to activity in pCi using a set of calibration standards with known activities, which were exposed and scanned on the same screen used for the aorta samples. The percentage injected dose (% ID) was calculated from dividing the activity (μCi) of the lesion by the injected activity. Activity density in % ID/m² was calculated and normalized by animal body weight to get % ID×kg/m². The areas of the lesions and the whole aortic arch in mm² were also recorded using the OptiQuant 5.0 software, and the percentage of lesion within the area of the aorta vessel wall was calculated.

Statistics. Data shown is either mean±SD of a single representative experiment or mean±SEM of at least 3 independent experiments performed in triplicates, and is indicated in the corresponding figure legends. Comparison between control and treatment was made using Student's t-test (p≦0.05) or comparison between groups by one-way ANOVA (p≦0.05) or two-way ANOVA (p≦0.05) using Prism GraphPad.

### Results

### Necroptotic Pathway is Activated in Unstable Atherosclerosis in Humans.

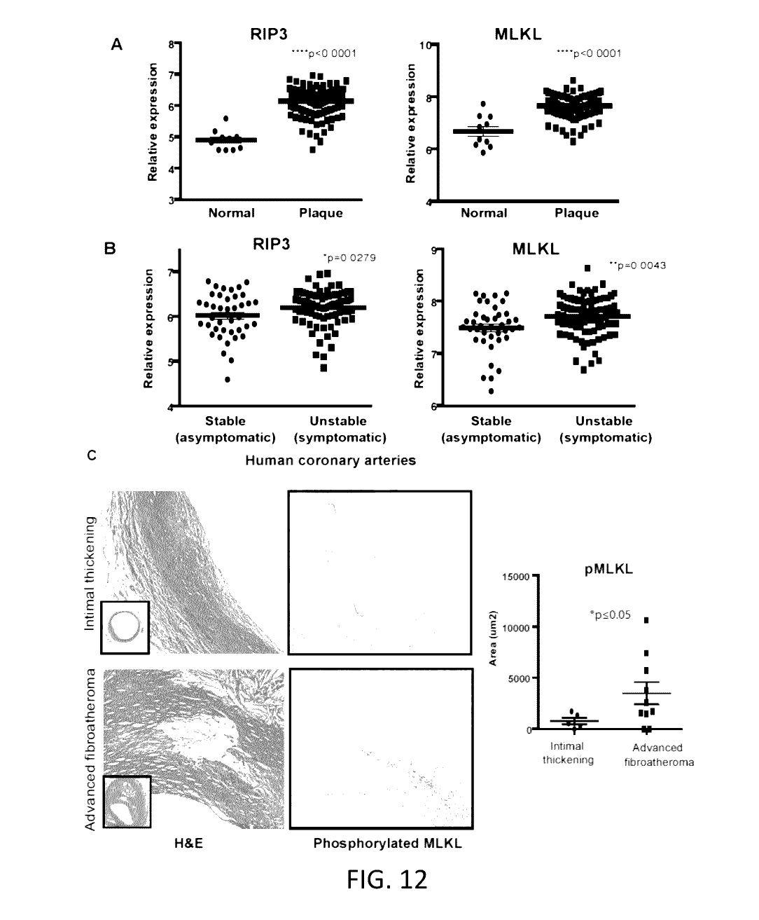

FIG. 12 illustrates an up-regulation of necroptotic genes in unstable atherosclerosis. Panel A shows mRNA expression of RIP3 and MLKL in carotid endarterectomies (plaque) or macroscopically disease-free control arteries (normal). Panel B shows RIP3 and MLKL mRNA expression in plaque samples from Panel A classified as asymptomatic (Stable) or symptomatic patients (Unstable) [from the Biobank of Karolinska Endarterectomies (BiKE)]. *p≦1.05, **p<0.01, ****p<0.0001 by Student's t-test. Panel C shows immunohistochemical analysis of phoshphorylated MLKL in human coronary arteries with early lesions with pathologic intimal thickening (n=5 arterial segments) and advanced fibroatheroma lesions (n=11 arterial segments). Graph depicts quantification of pMLKL positive area *p≦1.05.

Although necroptosis has been shown to promote lesion progression in an experimental mouse model of atherosclerosis (Lin et al., 2013), there is no suggestion that necroptosis is active in human plaques. The gene expression of RIP3 and MLKL in carotid plaques from a large biobank of patients was evaluated with atherosclerosis and in disease-free control arteries. Gene expression analysis showed a significant increase in expression of both RIP3 and MLKL mRNA in atherosclerotic plaques compared to normal arteries (p≦1.0001; FIG. 12, Panel A). Because it was hypothesized that necroptosis underlies lesion vulnerability, it was examined whether expression of necroptotic genes may be further increased in unstable versus stable atherosclerotic plaques. Gene expression analysis of plaques from individuals with symptomatic carotid disease (i.e. transient ischemic attack, minor stroke and/or amaurosis fugax) revealed a significant elevation of both RIP3 and MLKL gene expression compared to plaques from asymptomatic individuals (p≦1.05 and p<1.01, respectively, FIG. 12, Panel B). Traditional measures of cell death (e.g. TUNEL positivity) cannot distinguish between necroptotic and apoptotic cell death; however, the phosphorylation of MLKL—the last step in the execution of necroptosis—is considered to be the most definitive biomarker of necroptosis activity in vivo. To test if necroptosis was indeed activated in vascular lesions in humans, it was evaluated whether pMLKL could be detected in human coronary plaques with different stages of atherosclerotic lesions. Immunohistochemical analysis using an antibody that recognizes phosphorylated MLKL (pMLKL) showed that regions with advanced fibroatheroma lesions showed positive pMLKL staining in close proximity to the necrotic core, whereas within the same subject, early lesions (defined as pathologic intimal thickening) showed no positive pMLKL staining (FIG. 12, Panel C). Quantification of pMLKL positive area reveals that in subjects with advanced fibroatheromas there is a significant elevation of pMLKL compared to subjects with intimal thickening. This is the first evidence that the necroptotic pathway is associated with human vascular disease and the first report that phosphorylated MLKL is found within advanced atherosclerotic plaques, illustrating the contribution to lesion vulnerability.

### OxLDL Induces Necroptosis in Macrophages via RIP3.

To further understand the mechanisms by which necroptosis is activated in plaques, it was evaluated how atherogenic ligands trigger necroptosis by endogenous mechanisms in vitro. OxLDL is known to induce apoptosis, however little is known whether oxLDL or other atherogenic ligands found within the plaque can endogenously promote necroptosis in the absence of non-physiological apoptosis inhibitors (i.e. zVAD.fmk). Therefore, the degree of necroptotic cell death in macrophages in vitro in bone marrow derived macrophages (BMDMs) treated with oxidized LDL for 24 h was tested.

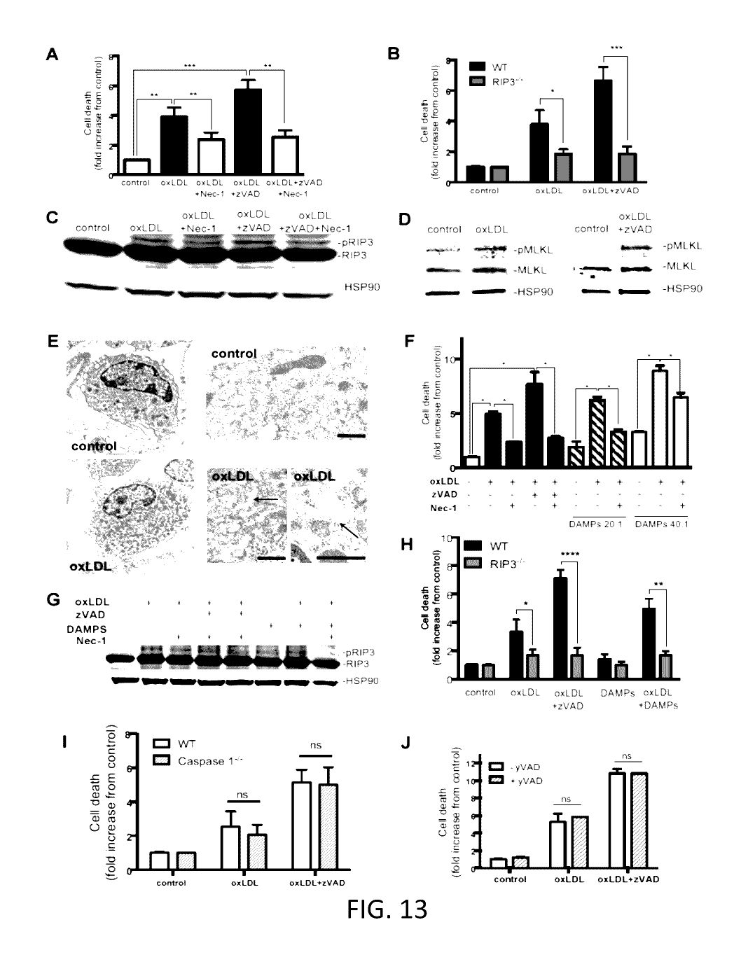

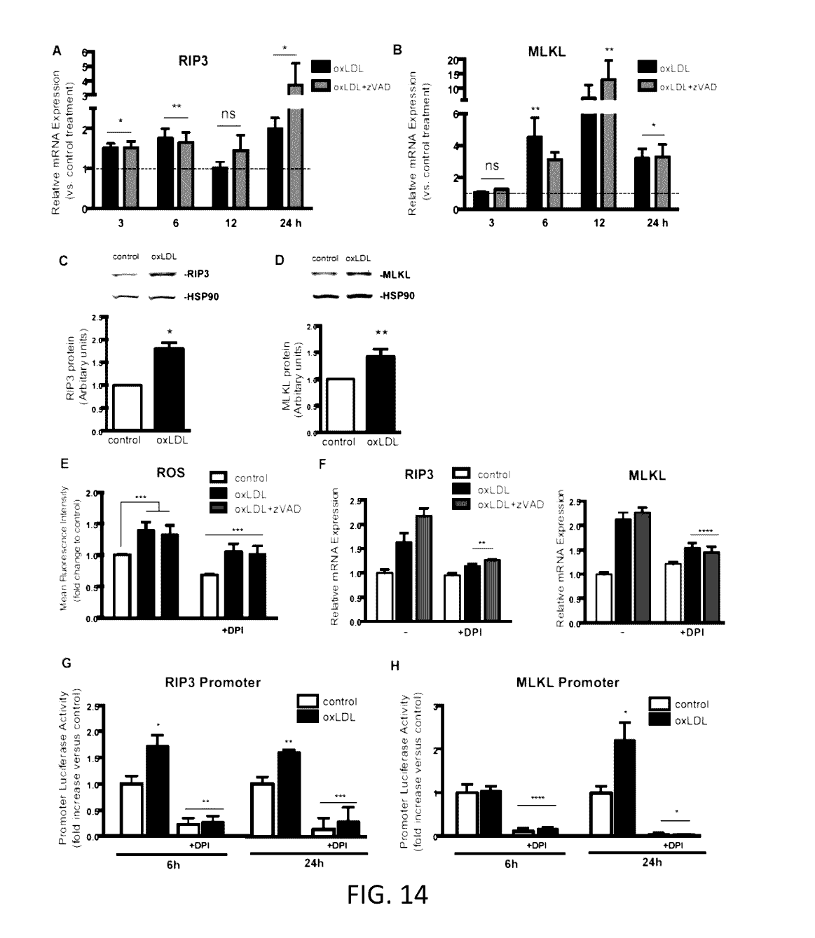

FIG. 13 shows that oxidized LDL induces necroptotic cell death in macrophages. (Panel A) Bone-marrow-derived macrophages (BMDMs) were treated with 100 μg/mL oxLDL±zVAD.fmk±Nec-1 for 24 h and LDH release in the media was measured. Data represents mean±SEM of 5 independent experiments. (Panel B) Cell death in response to oxLDL±zVAD.fmk in BMDMs from WT and Rip3−/− mice. (Panel C) Western blot analysis of RIP3 after treatment with oxLDL±zVAD.fmk ±Nec-1 for 8 hours. Band shift indicates phospho-RIP3. (Panel D) Western blot analysis of pMLKL after treatment with oxLDL for 12 h or oxLDL±zVAD.fmk for 8 h. (Panel E) Electron microscopy ultrastructural analysis of control and oxLDL-treated macrophages. Control macrophages had normal looking cytoplasm, whereas oxLDL-treated macrophages had electron-light zones (arrows) that were not observed in control macrophages. Scale bar=500 nm. (Panel F) BMDMs were subjected to freeze thaw to generate necrotic DAM Ps which were added to cells with or without oxLDL±Nec-1 for 24 h and cell death measured. (Panel G) Cell death in response to oxLDL±zVAD.fmk±necrotic freeze thaw DAMPs in BMDMs from WT and Rip3−/− mice. (Panel H) Western blot analysis of RIP3 after treatment with oxLDL±zVAD.fmk±Nec-1 with or without DAMPs for 8 hours. Band shift indicates phospho-RIP3. (Panel I) Cell death in BMDMs from WT and Casp1−/− mice in response to oxLDL±zVAD.fmk for 24 h. (Panel J)

Cell death in response to oxLDL±zVAD in BMDMs treated with or without the caspase-1 inhibitor zYVAD.fmk (20 uM). All data represents mean±SEM of at least 3 independent experiments. *p≦0.05, **p<0.01, ***p<0.001 by one-way ANOVA or two-way ANOVA.

Compared to unstimulated cells, oxLDL treatment resulted in a ˜4-fold induction of cell death in macrophages and this was significantly inhibited by co-treatment with the necroptosis inhibitor Nec-1 (3.9±0.6-fold vs. 2.3±0.4-fold, respectively; p≦0.01; FIG. 13, Panel A). Inhibition of apoptotic cell death with the pan caspase inhibitor zVAD.fmk significantly enhanced cell death in response to oxLDL, whereas Nec-1 treatment alone did not promote cell death (FIG. 13, Panel A). To test whether the induction of cell death is dependent upon RIP3 function, the induction of cell death by oxLDL was measured in both wild-type macrophages and macrophages deficient in RIP3 (Rip3−/−) and observed that Rip3−/− macrophages are resistant to cell death in response to both oxLDL and oxLDL+zVAD.fmk (FIG. 13, Panel B). As phospho-RIP3 and phospho-MLKL are required for the execution of necroptosis, the degree of phosphorylation of RIP3 and MLKL in response to oxLDL, was subsequently measured and it was observed that oxLDL significantly induced both RIP3 and MLKL phosphorylation in macrophages, which was dampened by Nec-1 (FIG. 13, Panels C and D). At the ultrastructural level, cells undergoing necroptosis have damaged plasma membrane integrity and translucent electron-light cytoplasm. Ultrastructural analysis of macrophages treated with oxLDL and oxLDL+zVAD.fmk using electron microscopy show the typical electron-light zones within the cytoplasm that are not found in control-treated cells (FIG. 13, Panel E). As smooth muscle cells (SMCs) form the fibrous cap and play an important role in the rupture of atherosclerotic plaques, it was investigated whether SMCs also undergo necroptosis in response to oxLDL, but it was found that neither oxLDL or oxLDL+zVAD.fmk significantly induced cell death in SMCs (data not shown). Taken together, these data demonstrate that oxLDL, a potent endogenous atherogenic ligand, can independently induce macrophages to undergo necroptosis in the absence of caspase inhibitors. This pathway may be activated under physiologic conditions in the vessel wall.

Within the atherosclerotic milieu, various stimuli are present in addition to oxLDL that can promote the inflammatory response and propagate lesion formation. These include cytokines, chemokines, reactive oxygen species, cholesterol crystals and other modified self-ligands, and together are termed damage-associated molecular patterns (DAMPs). DAMPs are released from cells during the process of necroptosis, where uncontrolled leakage of intracellular components occurs after membrane integrity is lost. It was therefore hypothesized that DAMPs released in response to necroptosis may exacerbate oxLDL-induced cell death. Indeed, co-incubation of DAMPs generated by either mechanical (i.e. freeze-thaw) or pharmacological (i.e. LPS+zVAD.fmk) methods together with oxLDL significantly induced cell death in macrophages, which could be inhibited by Nec-1 (FIG. 13, Panel F; p<0.05). Similar to what is observed with oxLDL alone, cell death in response to DAMPs is dependent upon RIP3, as Rip3−/− macrophages are resistant to cell death by oxLDL+DAMPs (FIG. 13, Panel G). Activation of RIP3 by phosphorylation is increased when macrophages are co-incubated with oxLDL and DAMPs, and can be inhibited by Nec-1 (FIG. 13, Panel H). These data indicate that in addition to oxLDL, DAMPs released from dying cells exacerbate necroptotic cell death and is dependent on RIP3 activation, suggesting that the multiple inflammatory ligands present in the atherosclerotic plaque may act concomitantly to promote necroptosis.

oxLDL is an inducer of the NLRP3 inflammasome, and can induce the expression and secretion of IL-1β. It was thus investigate whether the inflammasome was required for the induction of necroptosis by oxLDL. Macrophages from either wild-type or mice deficient in caspase-1 (Casp1−/−) had equivalent levels of cell death upon treatment with oxLDL (FIG. 13, Panel I). Similarly, co-treatment of macrophages with the caspase-1 inhibitor zYVAD.fmk did not reduce or prevent cell death in response to oxLDL (FIG. 13, Panel J). These data indicate that necroptotic cell death in response to oxLDL does not depend on the induction of caspase-1, unlike what is observed for IL-1β secretion.

### Oxidized LDL Induces Expression of Necroptotic Genes RIP3 and MLKL

OxLDL is an inflammatory ligand that induces the activation of pro-inflammatory signaling events, eliciting cytokine and chemokine production. Because oxLDL induces necroptosis independently of caspase inhibition, it was next sought to determine the mechanisms underlying the induction of necroptosis by oxLDL.

FIG. 14 shows that oxLDL induces the expression of RIP3 and MLKL. In Panels A and B, BMDMs treated with 100 ug/mloxLDL±zVAD.fmk for 3, 6, 12 or 24 h were analyzed for gene expression of RIP3 (Panel A) and MLKL (Panel B) by qPCR and compared to control treated cells. In Panels C and D, Western blot analysis of RIP3 (Panel C) and MLKL (Panel D) expression in BMDMs treated with oxLDL. Quantification below of at least 3 independent experiments. (Panel E) BMDMs were pre-incubated with 50 μM DPI and then treated with oxLDL or oxLDL+zVAD.fmk for 3 h prior to measuring reactive oxygen species (ROS) levels. Graph shows of mean±SD of technical triplicates and representative of at least 3 experiments. Statistical analysis was performed using two-way ANOVA, ***p<0.001. (Panel F) RIP3 or MLKL mRNA expression measured by qPCR in BMDMs treated with oxLDL±zVAD.fmk, in the presence or absence of pre-treatment with 50 μM DPI. (Panels G and H) RAW macrophages were transfected with RIP3-promoter (Panel G) or MLKL-promoter (Panel H) luciferase constructs and treated for 6 or 24 h with oxLDL in the presence or absence of pre-treatment with 50 uM DPI before luciferase expression was examined and expressed as promoter-luciferase activation normalized to control (no treatment). *p≦0.05, **p<0.01, ***p<0.001, p<0.0001 by one-way ANOVA.

The expression of genes involved in the necroptotic pathway in macrophages was measured and it was found that the treatment with oxLDL induced the expression of RIP3 and MLKL at both the mRNA (FIG. 14, Panel A and Panel B) and protein level (FIG. 14, Panels C and D). OxLDL can induce the production of reactive oxygen species (ROS), therefore it was tested if ROS production was critical for the induction of RIP3 and MLKL expression. It was confirmed that treatment of macrophages with oxLDL induced the production of ROS, which was inhibited by the ROS scavenger diphenyleneiodonium (DPI) (FIG. 14, Panel E). Pre-treatment of macrophages with DPI inhibited the induction of both RIP3 and MLKL mRNA expression by oxLDL and oxLDL+zVAD.fmk (FIG. 14, Panel F). To assess whether this increase in gene expression was due to direct activation of the promoter by oxLDL, we utilized a synthetic construct with the promoter region of either RIP3 or MLKL upstream of a luciferase reporter. In RAW macrophages transfected with the promoter constructs, oxLDL treatment significantly induced the activation of the RIP3 reporter after 6 h and 24 h, and the MLKL reporter after 24 h (FIG. 14, Panel G and H). Inhibition of ROS by treatment with DPI significantly blunted the promoter activity of RIP3 and MLKL indicative of a ROS-dependent activation of the promoter regions of RIP3 and MLKL by oxLDL (FIG. 14, Panel G and H). In addition to MLKL, PGAM5 can play a role in necroptosis by inducing mitochondrial damage. It was also found that PGAM5 was induced at the mRNA and protein level by oxLDL, and its expression was similarly inhibited by DPI. These results indicate that oxLDL directly induces the expression of RIP3 and MLKL at the mRNA and protein levels likely as a result of activation of the promoter regions of these genes by oxLDL signaling through ROS. Collectively, this data provides mechanistic insight into the observation in humans that necroptosis could be activated due to direct activation of the necroptotic program by atherogenic ligands in the plaque.

Imaging of Atherosclerosis in vivo Using Radiolabeled Nec-1

Given the evidence that the necroptotic pathway is activated in human plaques and its expression driven by atherogenic stimuli, we next test whether the necroptotic pathway could be targeted using molecular imaging to visualize its occurrence in atherosclerotic lesions in vivo. In this Example, an ¹²³I-labeled Nec-1 compound is tested for its localization to aortic lesions in Apoe−/− mice. Radiolabeled Nec-1 (¹²³I-Nec-1) was injected into Apoe−/− mice with established lesions and 2 hours later aortas were harvested and analyzed for radiotracer uptake.

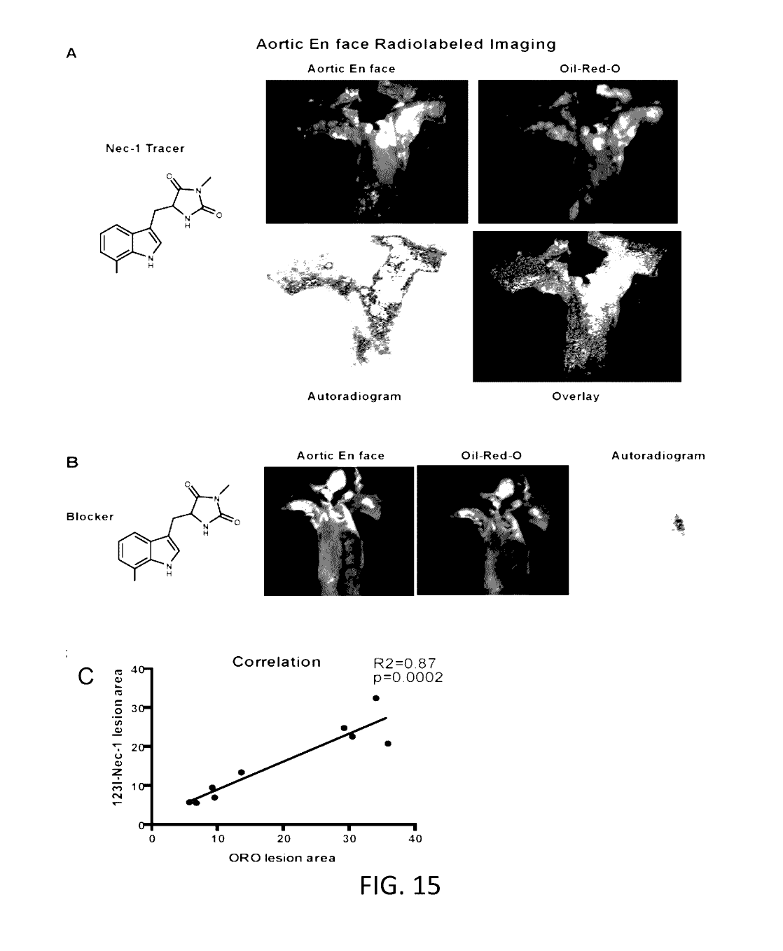

FIG. 15 shows radiolabeled Nec-1 can be utilized to visualize atherosclerotic plaques in Apoe−/− mice. (Panel A) Left: chemical structure of 7-¹²³I-Nec-1 tracer. Images of aortic en face (no stain and oil red-O [ORO] stain) and autoradiography from mice injected with ¹²³I-labelled Nec-1 tracer are shown. (Panel B) Mice were injected with non-radioactive Cl-Nec-1 compound 1.5 h prior to being injected with radiolabelled ¹²³I-Nec-1 tracer, subjected to autoradiography (images shown) and lesional uptake quantification (n=3 mice per group). (Panel C) Correlation of lesional uptake of ORO compared to ¹²³I-Nec-1 (n=9).

Autoradiography imaging revealed that ¹²³I-Nec-1 was primarily localized to areas of plaque accumulation in the en face aortic arch (FIG. 15, Panel A). Pre-administration of a cold non-radioactive Nec-1 compound significantly blocked aortic lesional uptake of ¹²³I-Nec-1 (FIG. 15, Panel 8), indicating that radiolabeled Nec-1 uptake is specific. When compared to oil red O (ORO) uptake within the aorta, which stains lipid-rich lesion areas, there was a significant correlation between lesion area measured by ¹²³I-Nec-1 and ORO (FIG. 15, Panel C), suggesting that this was localized to macrophage foam cells in plaques. These data demonstrate that necroptosis within atherosclerotic lesions can be targeted using a molecular imaging probe based on the small molecule inhibitor Nec-1 to visualize atherosclerotic lesion areas.

### Blocking Necroptosis in Established Lesions Reduces Plaque Size and Markers of Vulnerability

Active necroptosis was detected in advanced rupture-prone lesions in human subjects, therefore it was tested whether intervention using a small-molecule inhibitor of necroptosis could be used as a therapeutic strategy in mice with established atherosclerotic lesions.

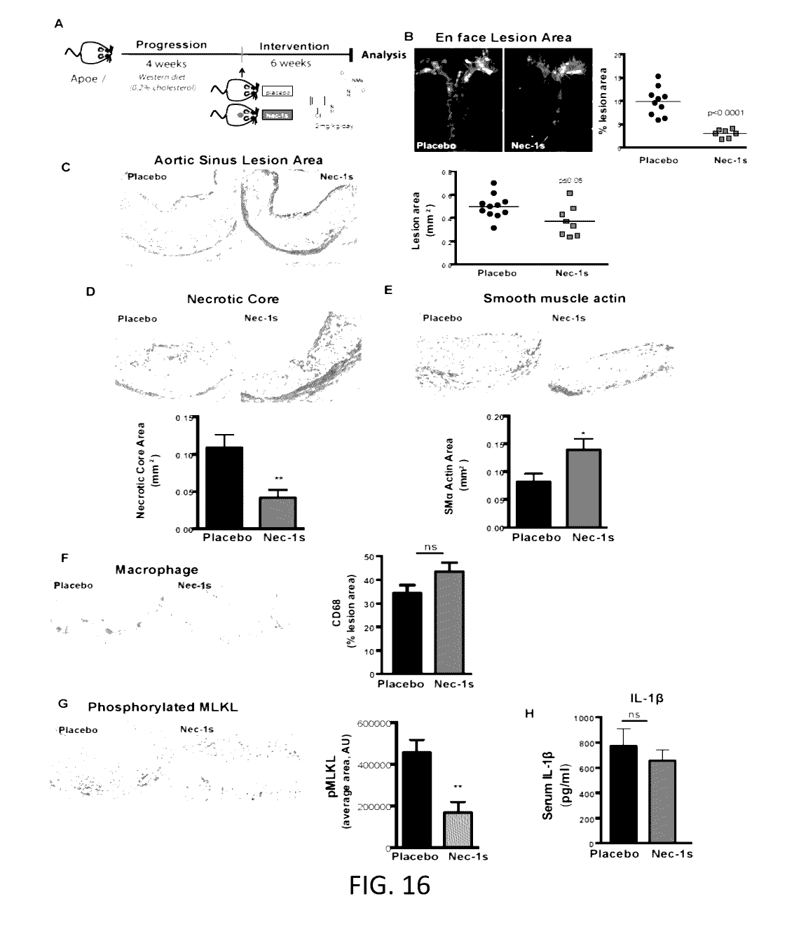

FIG. 16 shows that Nec-1 therapy decreases atherosclerotic lesion progression and markers of instability in ApoE−/− mice. (Panel A) ApoE−/− mice were fed a Western diet for 6 weeks prior to implantation of time-release pellets containing placebo or Nec-1s (2 mg/kg/day). After 4 additional weeks of Western diet feeding, mice were harvested for morphometric analysis of atherosclerosis. (Panel B) En face lesion area was measured in placebo and Nec-1s treated mice and is represented as lesion area as a % of total aorta area. (Panel C) Lesion area in the aortic sinus in placebo and Nec-1s treated mice is represented as total area in μm². (Panel D) Necrotic core area within aortic sinus lesions. (Panels E-G). Immunohistochemical staining of (Panel E) smooth muscle-α actin (smooth muscle cell marker) (Panel F) CD68 (macrophage marker) and (Panel G) phosphorylated MLKL was performed on aortic sinus lesions and was quantified with Image J. Representative images per group are shown. (Panel H) Serum IL-1β in mice from placebo or Nec-1 treated groups was measured at sacrifice by ELISA. *p≦0.05, **p<0.01 by Student t-test.

Necrostatin-1s (Nec-1) was formulated into a time-release pellet and delivered to ApoE−/− mice, which had been fed Western diet for 4 weeks to induce lesion development FIG. 16, Panel A). After 6 weeks of treatment with either Nec-1 or placebo with Western diet feeding, quantification of en face lesion area in the ascending and descending aorta revealed that Nec-1 treatment significantly reduced lesion burden compared to placebo treated mice by 68% (9.9±0.9% placebo versus 3.1±0.3% Nec-1; p≦0.0001; FIG. 16, Panel 8). In the aortic root, lesion area was reduced by 27% compared to controls (0.50±0.03 mm² placebo versus 0.37±0.05 mm² Nec-1; p≦1.05; FIG. 16, Panel C). Total plasma cholesterol and body weight were not affected by Nec-1 treatment (data not shown).

Intervention with an inhibitor of necroptosis can block the further progression of established lesions in mice with inflammatory atherosclerosis.

Next, it was evaluated whether intervention with Nec-1 altered markers of lesion instability; namely, necrotic core area and smooth muscle cell fibrous cap content. Evaluation of lesion characteristics demonstrate that Nec-1 reduced lesion necrotic core area by 62% compared to placebo (p≦1.01; FIG. 16, Panel D) and increased smooth muscle actin area by 70% (p≦1.05, FIG. 16, Panel E). Quantification of macrophage content within the lesions demonstrated that there was a slight but non-significant increase in CD68+ macrophage area with Nec-1 treatment, likely reflecting to the overall reduction in lesion size in the Nec-1 treated animals (34.4%±3.2 versus 43.5%±3.7 of lesion area, p=ns, FIG. 16, Panel F). It was next assessed whether necroptotic cell death was reduced in lesions from Nec-1 treated mice. The presence of phosphorylated MLKL was evaluated as a measure of necroptotic cell death in situ, and it was found that Nec-1 treatment reduced pMLKL positive area by 63% compared to placebo controls (p≦1.01; FIG. 16, Panel G). As RIP3 can also play a role in inflammasome activation and atherosclerosis progression, the levels of serum IL-1β were measured as a readout of inflammasome activation in these mice and found that serum levels of IL-1β were not different between the placebo or Nec-1 treated group (775±70 versus 658±84 μg/ml, p=ns; FIG. 16, Panel H). These results demonstrate that in mice with established atherosclerotic lesions, intervention with a pharmacological inhibitor of necroptosis can prevent further lesion progression and reduce markers of plaque instability (i.e. necrotic core, necroptotic cell death), suggesting that necroptosis underlies both the development and vulnerability of atherosclerotic plaques and can be targeted to reduce these markers of lesion instability.

### Discussion

In this Example it is show that necroptosis, an emerging pathway underlying inflammatory cell death in many pathologies, is active within the necrotic core in humans with advanced atherosclerosis and can be targeted for therapeutic and diagnostic interventions in mice. This is the first to demonstration that the necroptotic pathway is increased in vascular disease in humans, where MLKL is phosphorylated in advanced necrotic lesions. It is shown that necroptosis can be targeted with long-term therapeutic inhibition in a mouse model of established atherosclerosis to reduce lesion size and, importantly, reduce markers of plaque vulnerability. Moreover, using ex vivo molecular imaging, it is shown that necroptosis can be used as a diagnostic tool to visualize this process in atherosclerotic lesions. Mechanistically, it was demonstrated that the sterile ligand oxLDL, an inflammatory form of LDL that is abundant within developing atheroma, is sufficient to induce necroptotic cell death in macrophages via direct activation of the RIP3 and MLKL promoter. Although oxLDL is known to induce apoptosis, this is the first demonstration that oxLDL can directly induce necroptotic cell death in the absence of synthetic caspase inhibition. Overall, this Example offers compelling evidence that necroptosis contributes to atherosclerosis lesion vulnerability in humans and offers the first mechanistic insight into how atherogenic ligands are driving this form of inflammatory cell death.

In advanced human atherosclerotic lesions, cells with necrotic morphology are more abundant than with apoptotic morphology, suggesting that other forms of cell death beyond apoptosis endure in the advanced plaque and promote lesion destabilisation. This Example suggests that so long as oxLDL persists within the plaque, macrophages undergo necroptotic cell death, releasing DAMPs into the extracellular space to serve as a feed-forward loop to exacerbate the extensive necrotic core found in advanced lesions.

This Example shows that oxLDL induces the expression of necroptotic genes RIP1, RIP3 and MLKL and, importantly, promotes the upregulation and phosphorylation of RIP3 protein—a requirement for the assembly of the RIP1-RIP3 complex and subsequent necroptosis. In addition, this data shows that atherogenic ligands can induce the phosphorylation of MLKL, and this is active within the atherosclerotic plaque. This Example offers mechanistic insight into why Rip3−/− mice are protected from the development of atherosclerosis, suggesting that endogenous ligands present within the atherosclerotic plaque are directly responsible for the upregulation of RIP3, which is sufficient to drive necroptosis and lesion progression. This Example shows that oxLDL triggers necroptosis independently from caspase-8 inhibition by direct transcriptional upregulation of necroptotic cell genes RIP3 and MLKL. It is shown in this Example that oxLDL induces mRNA and protein expression of RIP3 and MLKL and is sufficient to activate necroptosis which is blocked upon scavenging cellular ROS. The induction of necroptosis by oxLDL is independent of inflammasome activation, as cells deficient in caspase-1 or treated with caspase-1 inhibitors undergo necroptotic cell death in response to oxLDL to the same degree as wild-type or untreated cells. This evidence could explain why gene expression of RIP3 and MLKL are positively associated with unstable atherosclerotic lesions compared to both stable lesions and healthy arteries. An improved understanding how atherogenic ligands induce the expression of RIP3 and MLKL could assist in the rationale design of future therapeutics that block this mechanism to reduce atherosclerosis lesion vulnerability.

This Example establishes the potential for the therapeutic treatment of established atherosclerosis with inhibitors of the necroptotic pathway. Within the atherosclerotic plaque, RIP3 contributes to inflammasome activation, which could be inhibited by blocking RIP1-RIP3 activation with Nec-1. Differences in serum IL-1β levels in mice treated with Nec-1 were not observed, suggesting that at doses used in the current Example, Nec-1 treatment did not impair inflammasome activation and thus likely did not contribute to the reduction in lesion size observed in the Nec-1 treated group

We observed a reduction in pMLKL in Nec-1 treated mice, specifically indicating that necroptotic death is reduced in mice treated with Nec-1. This Example illustrates that Nec-1 can be used in a long-term therapeutic application, and opens the door for next-generation necroptosis inhibitors with improved specificity and stability for the treatment of chronic inflammatory diseases like atherosclerosis.

Current clinical practice relies on invasive angiography to visualize plaques in patients with atherosclerosis, which provides limited detail of plaque size but no insight into plaque inflammation or vulnerability. Imaging by positron emission tomography (PET) or single photon emission computed tomography (SPECT) provides the advantage of being non-invasive, with the ability of detecting specific molecular processes that provide insight into the pathology of the plaque when specific molecular tracers are used. ¹⁸F-fluorodeoxyglucose (FDG) is a widely-used nuclear tracer to detect inflammation tissues, as macrophages take up FDG at a higher rate than non-inflammatory cells, making FDG a surrogate of plaque inflammation. FDG-PET has supported the concept that molecular imaging of dominant pathways within the atherosclerotic plaque correlates with lesion vulnerability and thus may be of added value when assessing overall cardiovascular risk.

However, there remains a need to develop novel radiotracers that can detect coronary artery inflammation without the complication of myocardial uptake, and with less sensitivity to metabolic characteristics of the patient (i.e. high fasting blood glucose).

In this Example, it is shown that ¹²³I-Nec-1 co-localizes to the atherosclerotic plaques in Apoe−/− mice by autoradiographic imaging and strongly correlated with that of traditional ORO uptake. Although the uptake of the Nec-1 radiotracer was only measured ex vivo in the current study, similar radiotracers can be developed and labeled with PET radionuclides and allow Nec-1 to be used in the future for non-invasive PET imaging in animal models, with the ultimate goal of detecting atherosclerotic lesions in patients with disease.

### In Summary.

This Example shows that necroptosis activation is associated with necrotic core formation in advanced human atheromas, and supports the therapeutic targeting of necroptosis to reduce lesion progression and plaque vulnerability. This Example shows that targeting of necroptosis can be used as a diagnostic tool using either nuclear imaging and/or biomarker expression to better identify patients at the highest risk for lesion rupture. These findings offers mechanistic insight into how atherogenic ligands drive necrotic core development to better devise therapies that specifically inactivate the necroptotic pathway to directly treat the underlying causes of clinically vulnerable atherosclerosis.

### Example 4

Synthesis of Nec-3 Analogs: ¹²³I-Nec-3 SPECT Compound Localizes with Atherosclerotic Lesions ex vivo

Novel imaging agents enable the visualization of necroptosis in vascular disease, where necroptosis is known to play a role in disease pathology. Such tracers are of use in visualizing vulnerable atherosclerotic plaques in particular. New Nec-3 agents are described in this Example for use in PET and SPECT tracers.

Synthesis, characterization and biodistribution of three Nec-3 tracers

Three ¹²³I labeled Nec-3 derivatives (R-isomers, 3′-¹²³I-Nec-3R, 4′-¹²³I-Nec-3R, and 8-¹²³I-Nec-3R) were prepared according to Scheme 2.

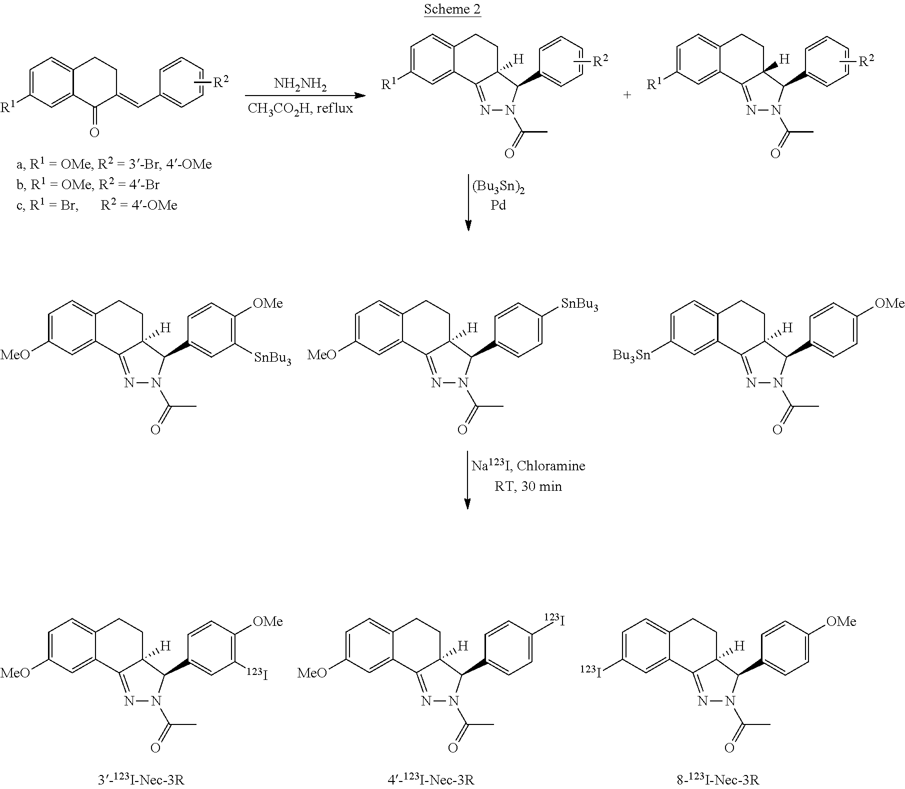

In Scheme 2, synthetic routes for preparation of these three ¹²³I-labelled Nec-3 tracers is represented. Tracers were prepared by iododestannylation reactions through tributyltin precursors. The radiochemical purity was >95% after HPEC purification. Cold compounds for all three tracers were prepared and characterized by NMR and mass spectrometry to confirm the molecular structures.

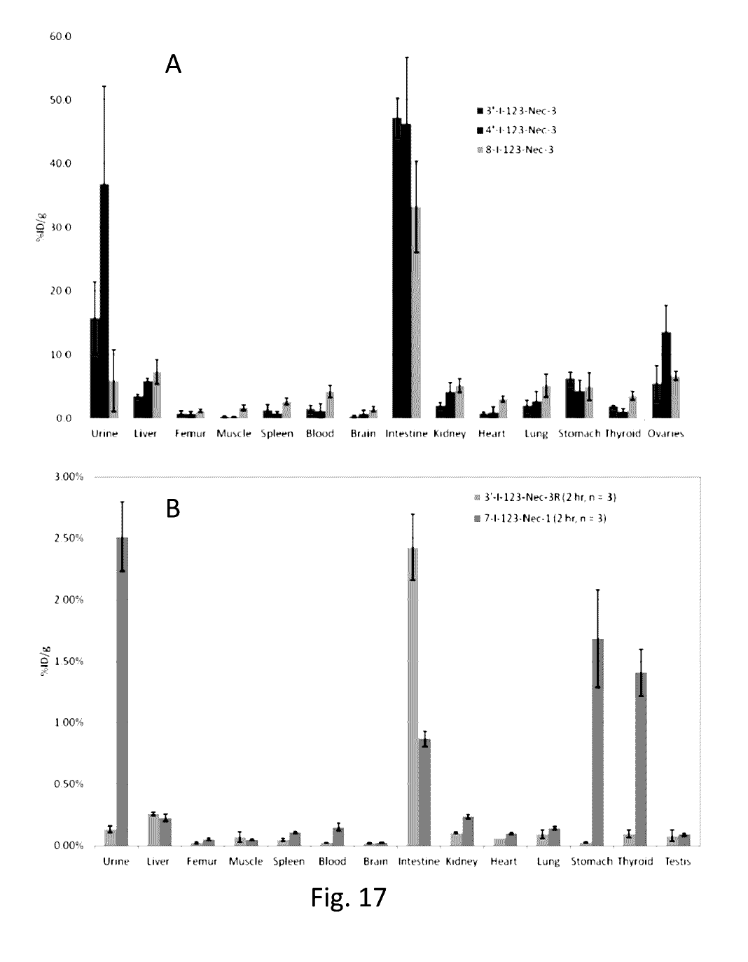

All three tracers were stable in rat serum at 37° C. for 24 hr. Biodistribution studies in mice and rats indicated significant clearance of the tracers from the renal and gastrointestinal system. FIG. 17, Panel A shows biodistribution of the three labeled Nec-3 tracers in female C57BL/6 mice at 2 hours, post injection. FIG. 17, Panel B shows a comparison of biodistribution of 1231-labeled Nec-1 and Nec-3 tracers in male Sprague-Dawley rats at 2 hours post injection (n=3 for each group). The organ uptake values are reported as percentage of injected dose per gram (% ID/g) and are presented as mean±SD.

### Evaluation in Atherosclerosis Mouse Model

Similar to the evaluation of the ¹²³I-Nec-1 tracer, we used ApoE−/− mice to evaluate the uptake of the Nec-3 tracers in atherosclerotic lesions. The mice were injected with ˜1 mCi of tracer, euthanized at 2 hr post injection, and the aortas were subjected to en face, Oil Red 0 staining and autoradiography imaging analysis. Atherosclerotic lesions showed significant uptake of 3′-¹²³I-Nec-3R by autoradiography.

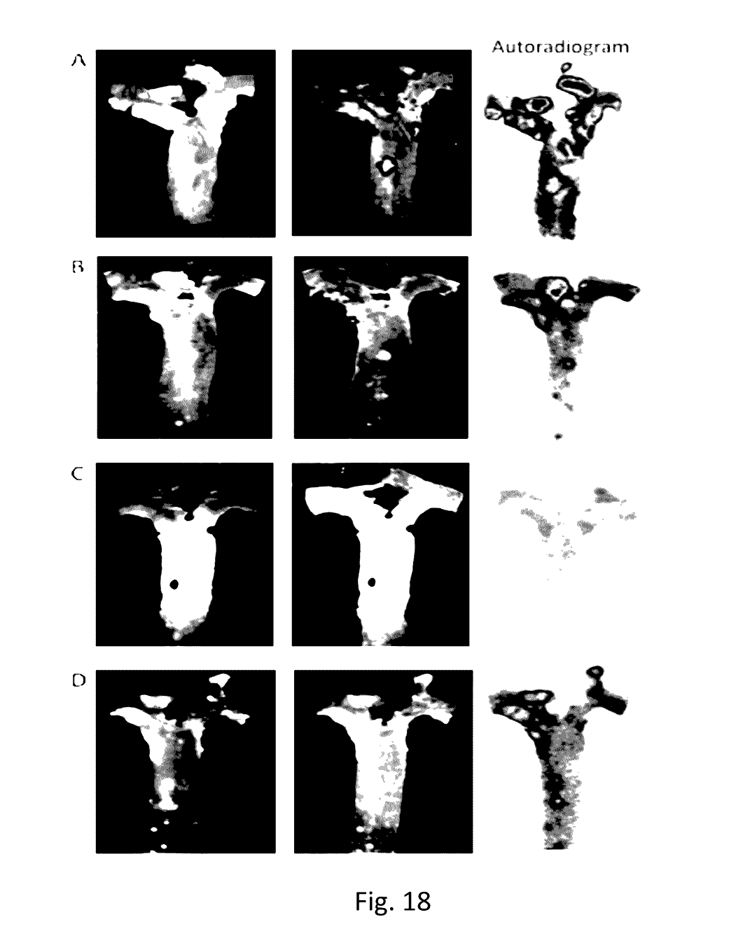

FIG. 18 shows en face (first column), Oil Red 0 (second column), and autoradiography (third column) aortic images from a 12-month old ApoE−/− mouse injected with 3′-¹²³I-Nec-3R (Panel A), a 4-month old ApoE−/− mouse following 2-months of consuming a Wester high fat diet (HFD), injected with 3′-¹²³I-Nec-3R (Panel B), and 4-month old wild type C57BL/6 mouse injected with 3′-¹²³I-Nec-3R (Panel C), and a 4-month old ApoE−/− mouse injected with 8-¹²³I-Nec-3R following 2 months of a high fat diet (Panel D).

FIG. 18, Panels A & B show that uptake is high, in comparison with wild-type mice with no atherosclerotic lesions (Panel C), where there is low tracer uptake visualized by autoradiography. Another Nec-3 tracer 8-¹²³I-Nec-3R also showed significant lesion uptake in ApoE−/− mice (Panel D). This example shows the usefulness of these labeled Nec compounds for visualization and localization within lesion areas.

These labels permit identification of unstable plaques through visualization of localized regions of label.

### Example 5

### ¹⁸F-Labelled Nec-1 and Nec-3 Compounds

Compounds labelled with ¹⁸F may be used in PET imaging. The following compounds may be utilized: Formula V and Formula VI being ¹⁸F-Nec-1 compounds and Formulae VII, VIII, IX, and X being ¹⁸F-Nec-3 compounds. Radiolabeling with ¹⁸F can be achieved through a deoxyfluorination reagent from a phenol precursor; halogen exchange reactions with the brominated precursors; fluorination of the triflate or tributyltin precursors. Cold fluorinated Nec-1 and Nec-3 compounds can be prepared.

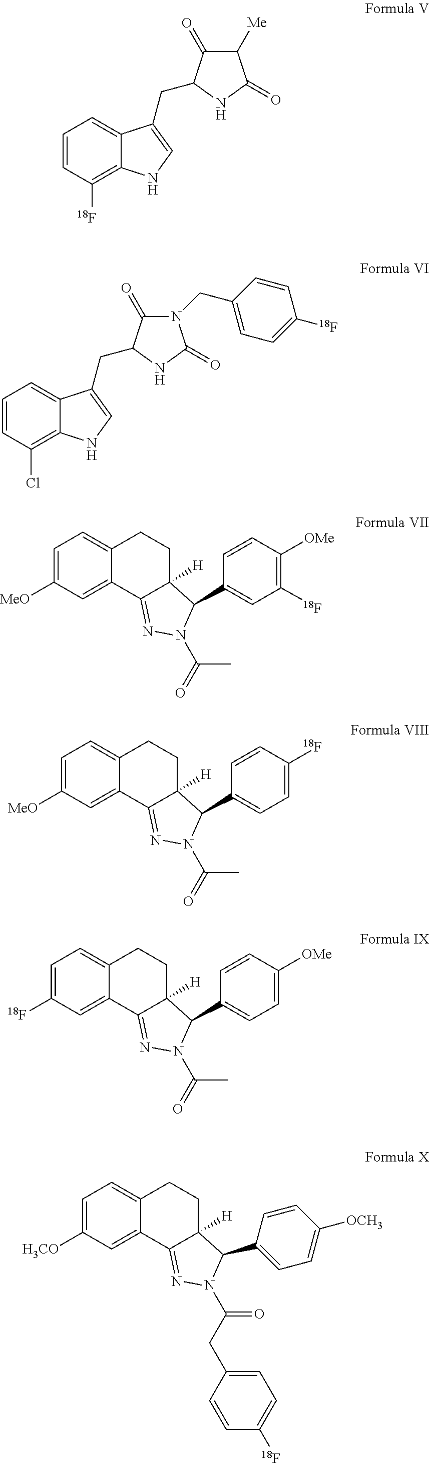 [SMILES: CC1C(=O)NC(Cc2c[nH]c3c([18F])cccc23)C1=O ; O=C1NC(Cc2c[nH]c3c(Cl)cccc23)C(=O)N1Cc1ccc([18F])cc1 ; COc1ccc2c(c1)C1=NN(C(C)=O)[C@H](c3ccc(OC)c([18F])c3)[C@@H]1CC2 ; COc1ccc2c(c1)C1=NN(C(C)=O)[C@H](c3ccc([18F])cc3)[C@@H]1CC2 ; COc1ccc([C@@H]2[C@@H]3CCc4ccc([18F])cc4C3=NN2C(C)=O)cc1 ; COc1ccc([C@@H]2[C@@H]3CCc4ccc(OC)cc4C3=NN2C(=O)Cc2ccc([18F])cc2)cc1]

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

All references noted herein are incorporated by reference.

U.S. Publication No. U.S. 2014/0357570 A1

U.S. Publication No. U.S. 2013/0158024 A1

U.S. Publication No. U.S. 2014/0323489 A1
Bauernfeind, F., et al. Cutting edge: reactive oxygen species inhibitors block priming, but not activation, of the NLRP3 inflammasome. J Immunol 187, 613-617 (2011).
Cai, Z., et al. Plasma membrane translocation of trimerized MLKL protein is required for TNF-induced necroptosis. Nature cell biology 16, 55-65 (2014).
Christofferson, D. E., and Yuan, J. (2010) Necroptosis as an alternative form of programmed cell death. Curr Opin Cell Biol. 22, 263-268.
Degterev, A., Huang, Z., Boyce, M., Li, Y., Jagtap, P., Mizushima, N., Cuny, G. D., Mitchison, T., Moskowitz, M., and Yuan, J. (2005) Chemical inhibitor of nonapoptotic cell death with therapeutic potential for ischemia brain injury. Nat Chem Biol. 1, 112-119.
Gan, W. et al., (2014) Potential SPECT imaging radiotracers targeting necroptosis: 1-123 labeled necrostatin-1 and necrostatin-3 derivatives. J Nucl Med; 55 (Supplement 1):332.
Gao, M. et al. (2010) Synthesis of carbon-11-Tabled tricyclic necroptosis inhibitors as new potential PET agents for imaging of tumor necrosis factor a (C). Appl Radiat isotopes 68. 2010. 1950-1958.
Janin, Y. L., Huel, C., Flad, G., and Thirot, S. (2002) Methyl orthocarboxylates as methylating agents of heterocycles. Eur J Org Chem. 1763-1769.
Klapars, A., and Buchwald, S. L. (2002) Copper-catalyzed halogen exchange in aryl halides: an aromatic Finkelstein reaction. J Am Chem Soc. 124, 14844-14845.
Karunakaran, D., et al. (2013) Protein kinase C controls vesicular transport and secretion of apolipoprotein E from primary human macrophages. J Biol Chem 288, 5186-5197.
Lin, J., et al. A Role of RIP3-Mediated Macrophage Necrosis in Atherosclerosis Development. Cell Reports 3, 200-210, Jan. 31, 2013.
Linkermann, A., and Green, D. R. (2014) Necroptosis. N Engl J Med. 370, 455-465.
Matter, C. M., et al. 18F-choline images murine atherosclerotic plaques ex vivo. Arterioscler Thromb Vasc Biol 26, 584-589 (2006).
Peled, M. & Fisher, E.A. Dynamic Aspects of Macrophage Polarization during Atherosclerosis Progression and Regression. Frontiers in immunology 5, 579 (2014).
Perisic, L., et al. Profiling of atherosclerotic lesions by gene and tissue microarrays reveals PCSK6 as a novel protease in unstable carotid atherosclerosis. Arterioscler Thromb Vasc Biol 33, 2432-2443 (2013).
Razuvaev, A., et al. Correlations between clinical variables and gene-expression profiles in carotid plaque instability. Eur J Vasc Endovasc Surg 42, 722-730 (2011).
Robinson, N., et al. Type I interferon induces necroptosis in macrophages during infection with Salmonella enterica serovar Typhimurium. Nat Immunol 13, 954-962 (2012).
Smith, C. C., and Yellon, D. M. (2011) Necroptosis, necrostatins and tissue injury. J Cell Mol Med. 15, 1797-1806.
Takahashi, N., Duprez, L., Grootjans, S., Cauwels, A., Nerinckx, W., DuHadaway, J. B., Goossens, V., Roelandt, R., Van Hauwermeiren, F., Libert, C., Declercq, W., Callewaert, N., Prendergast, G. C., Degterev, A., Yuan, J., and Vandenabeele, P. (2012) Necrostatin-1 analogues: critical issues on the specificity, activity and in vivo use in experimental disease models. Cell Death Dis. 3, e437.
Teng, X., Degterev, A., Jagtap, P., Xing, X., Choi, S., Denu, R., Yuan, J., and Cuny, G. D. (2005) Structure-activity relationship study of novel necroptosis inhibitors. Bioorg Med Chem Lett. 15, 5039-5044.
Vandenabeele, P., Galluzzi, L., Vanden Berghe, T. & Kroemer, G. Molecular mechanisms of necroptosis: an ordered cellular explosion. Nature reviews. Molecular cell biology 11, 700-714 (2010).
Xie et al. Structural Basis of RIP1 Inhibition by Necrostatins. Structure 21, 493-499, Mar. 5, 2013.
Zhao, Y., et al. Comparison of 99mTc-annexin A5 with 18F-FDG for the detection of atherosclerosis in ApoE−/− mice. European journal of nuclear medicine and molecular imaging 34, 1747-1755 (2007).
Zhou, W., and Yuan, J. (2014) Necroptosis in health and diseases. Semin Cell Dev Biol. 35, 14-23.

1. A method of detecting an atherosclerotic plaque vulnerable to rupture in a subject comprising providing to the subject a labelled necrostatin or a derivative thereof, and visualizing the label, wherein a localization of the label in a plaque indicates the plaque is vulnerable to rupture.

2. The method of claim 1, wherein the label is a radiolabel.

3. The method of claim 1, wherein the labelled necrostatin comprises necrostatin-1 (Nec-1), necrostatin-3 (Nec-3), necrostatin-4 (Nec-4), necrostatin-5 (Nec-5) or necrostatin-7 (Nec-7).

4. The method of claim 3, wherein the labelled necrostatin comprises:
¹²³I—labelled or ¹⁸F-labelled Nec-1,
¹²³I—labelled or ¹⁸F-labelled Nec-3, or a derivative thereof.

5. The method of claim 4, wherein the labelled necrostatin comprises 7-¹²³I-O-Nec-1.

6. The method of claim 1, wherein the localization of label in a plaque indicates an active necrotic plaque comprising macrophages.

7. The method of claim 1, wherein the labelled necrostatin comprises a Nec-1, Nec-3, or a derivative thereof for single photon emission computed tomography (SPECT) imaging.

8. The method of claim 1, wherein the labelled necrostatin comprises an ¹⁸F-labelled Nec-1, Nec-3 or a derivative thereof for positron emission tomography (PET) imaging.

9. The method of claim 1, wherein the extent of the localization of the label in the plaque of the subject is compared with the accumulation of the label at a level indicative of atherosclerosis.

10. The method of claim 1, wherein the extent of the localization of the label in the plaque of the subject is compared with the accumulation of the label at a level indicative of a ruptured plaque.

11. A method of detecting and treating an atherosclerotic plaque vulnerable to rupture in a subject comprising:
providing to the subject a labelled necrostatin or a derivative thereof;
visualizing the label, wherein a localization of the label in a plaque indicates the plaque is vulnerable to rupture; and
providing a necroptosis inhibitor or derivative thereof to the subject when the visualizing indicates that the plaque is vulnerable to rupture.

12. The method of claim 11, wherein the necroptosis inhibitor is Nec-1.

13. The method of claim 11, wherein the necroptosis inhibitor or derivative thereof comprises a conjugate of necrostatin linked to an anti-inflammatory agent for targeted delivery of the anti-inflammatory agent to an atherosclerotic plaque.

14. The method of claim 13, wherein the anti-inflammatory agent reduces or inhibits RIP1, RIP3, or MLKL expression.

15. The method of claim 11, wherein the necroptosis inhibitor comprises a small molecule, a microRNA (miRNA), a small interfering RNA (sRNA), or a short hairpin RNA (shRNA).

16. The method of claim 11, wherein the necroptosis inhibitor reduces production, activity or expression of a RIP3 or a RIP1 kinase, or of MLKL in macrophages.

17. The method of claim 11, wherein the subject has atherosclerosis.

18. The method of claim 11, wherein treating the atherosclerotic plaque prevents a thrombotic event, or delays or reverses the progression of the plaque.

19. The method of claim 11, wherein the necroptosis inhibitor reduces production or activity of the RIP3 kinase in macrophages.

20. The method of claim 11, wherein the subject has consumed a high fat diet.
